(19) United States
(12) Patent Application Publication
(10) Pub. No.: US 2016/0317060 A1
(43) Pub. Date: Nov. 3, 2016

(54) Finger Ring with Electromagnetic Energy Sensor for Monitoring Food Consumption

(71) Applicant: Robert A. Connor, Forest Lake, MN (US)

(72) Inventor: Robert A. Connor, Forest Lake, MN (US)

(73) Assignee: Medibotics LLC, Forest Lake, MN (US)

(21) Appl. No.: 15/206,215

(22) Filed: Jul. 8, 2016

Related U.S. Application Data
(63) Continuation-in-part of application No. 14/948,308, filed on Nov. 21, 2015.
Application No. 14/948,308 is a continuation-in-part of application No. 13/901,099, filed on May 23, 2013, now Pat. No. 9,254,099.
Application No. 13/901,099 is a continuation-in-part of application No. 14/132,292, filed on Dec. 18, 2013, now Pat. No. 9,442,100.
Application No. 14/132,292 is a continuation-in-part of application No. 14/449,387, filed on Aug. 1, 2014.
Application No. 14/449,387 is a continuation-in-part of application No. 14/951,475, filed on Nov. 24, 2015.
Application No. 14/951,475 is a continuation-in-part of application No. 13/901,131, filed on May 23, 2013.
Application No. 13/901,131 is a continuation-in-part of application No. 14/071,112, filed on Nov. 4, 2013.
Application No. 14/071,112 is a continuation-in-part of application No. 14/623,337, filed on Feb. 16, 2015.
(60) Provisional application No. 62/245,311, filed on Oct. 23, 2015.
(60) Provisional application No. 62/349,277, filed on Jun. 13, 2016.

(51) Int. Cl. A61B 5/053; A61B 5/00

(52) U.S. Cl. CPC A61B 5/053; A61B 5/6802; A61B 5/42; A61B 5/7271; A61B 5/0022; A61B 5/4866; A61B 5/6804; A61B 5/0075

(57) ABSTRACT

This invention is finger ring with an electromagnetic energy sensor for monitoring a person's food consumption. In an example, this device can monitor a person's food consumption by measuring changes in the electromagnetic impedance, resistance, conductivity, or permittivity of finger tissue. In an example, this finger-worn device can comprise an electromagnetic resonator between an electromagnetic energy emitter and an electromagnetic energy receiver.

### CROSS-REFERENCE TO RELATED APPLICATIONS

This patent application:

(1) is a continuation in part of U.S. patent application Ser. No. 14/948,308 by Robert A. Connor entitled “Spectroscopic Finger Ring for Compositional Analysis of Food or Other Environmental Objects” filed on Nov. 21, 2015 which, in turn, is: (a) a continuation in part of U.S. patent application Ser. No. 13/901,099 (now U.S. Pat. No. 9,254,099) by Robert A. Connor entitled “Smart Watch and Food-Imaging Member for Monitoring Food Consumption” filed on May 23, 2013; (b) a continuation in part of U.S. patent application Ser. No. 14/132,292 by Robert A. Connor entitled “Caloric Intake Measuring System using Spectroscopic and 3D Imaging Analysis” filed on Dec. 18, 2013; and (c) a continuation in part of U.S. patent application Ser. No. 14/449,387 by Robert A. Connor entitled “Wearable Imaging Member and Spectroscopic Optical Sensor for Food Identification and Nutrition Modification” filed on Aug. 1, 2014;

(2) is also a continuation in part of U.S. patent application Ser. No. 14/951,475 by Robert A. Connor entitled “Wearable Spectroscopic Sensor to Measure Food Consumption Based on Interaction Between Light and the Human Body” filed on Nov. 24, 2015 which, in turn: (a) is a continuation in part of U.S. patent application Ser. No. 13/901,131 by Robert A. Connor entitled “Smart Watch and Food Utensil for Monitoring Food Consumption” filed on May 23, 2013; (b) is a continuation in part of U.S. patent application Ser. No. 14/071,112 by Robert A. Connor entitled “Wearable Spectroscopy Sensor to Measure Food Consumption” filed on Nov. 4, 2013; (c) is a continuation in part of U.S. patent application Ser. No. 14/623,337 by Robert A. Connor entitled “Wearable Computing Devices and Methods for the Wrist and/or Forearm” filed on Feb. 16, 2015; and (d) claims the priority benefit of U.S. provisional patent application 62/245,311 by Robert A. Connor entitled “Wearable Device for the Arm with Close-Fitting Biometric Sensors” filed on Oct. 23, 2015; and

(3) claims the priority benefit of U.S. provisional patent application 62/349,277 by Robert A. Connor entitled “Glucowear™ System for Monitoring and Managing Intra-body Glucose Levels” filed on Jun. 13, 2016.

The entire contents of these related applications are incorporated herein by reference.

### FEDERALLY SPONSORED RESEARCH

Not Applicable

### SEQUENCE LISTING OR PROGRAM

Not Applicable

### BACKGROUND

1. Field of Invention

This invention relates to wearable technology for monitoring food consumption.

2. Introduction

The United States population has some of the highest prevalence rates of obese and overweight people in the world. Further, these rates have increased dramatically during recent decades. In the late 1990's, around one in five Americans was obese. Today, that figure has increased to around one in three. It is estimated that around one in five American children is now obese. The prevalence of Americans who are generally overweight is estimated to be as high as two out of three. Despite the considerable effort that has been focused on developing new approaches for preventing and treating obesity, the problem is growing. There remains a serious unmet need for new ways to help people to moderate their consumption of unhealthy food, better manage their energy balance, and lose weight in a healthy and sustainable manner.

Since many factors contribute to obesity, good approaches to weight management are comprehensive in nature. Proper nutrition and management of caloric intake are key parts of a comprehensive approach to weight management. Consumption of “junk food” that is high in simple sugars and saturated fats has increased dramatically during the past couple decades, particularly in the United States. This has contributed significantly to the obesity epidemic. For many people, relying on willpower and dieting is not sufficient to moderate their consumption of unhealthy “junk food.” The results are dire consequences for their health and well-being. The invention that is disclosed herein directly addresses this problem by helping a person to monitor and measure their nutritional intake. The invention that is disclosed herein is an innovative technology that can be a key part of a comprehensive system to help a person reduce their consumption of unhealthy types and/or quantities of food.

### REVIEW OF THE RELEVANT ART

U.S. patent application 20140061486 by Bao et al. entitled “Spectrometer Devices” discloses a spectrometer including a plurality of semiconductor nanocrystals which can serve as a personal UV exposure tracking device. Other applications include a smartphone or medical device wherein a semiconductor nanocrystal spectrometer is integrated.

U.S. patent application 20150148632 by Benaron entitled “Calorie Monitoring Sensor and Method for Cell Phones, Smart Watches, Occupancy Sensors, and Wearables” discloses a sensor for calorie monitoring in mobile devices, wearables, security, illumination, photography, and other devices and systems which uses an optional phosphor-coated broadband white LED to produce broadband light, which is then transmitted along with any ambient light to a target such as the ear, face, or wrist of a living subject. Calorie monitoring systems incorporating the sensor as well as methods are also disclosed. U.S. patent application 20150148636 by Benaron entitled “Ambient Light Method for Cell Phones, Smart Watches, Occupancy Sensors, and Wearables” discloses a sensor for respiratory and metabolic monitoring in mobile devices, wearables, security, illumination, photography, and other devices and systems that uses a broadband ambient light. The sensor can provide identifying features of type or status of a tissue target, such calories used or ingested.

U.S. Pat. No. 9,254,099 by Connor entitled “Smart Watch and Food-Imaging Member for Monitoring Food Consumption” discloses a device and system for monitoring a person's food consumption comprising: a wearable sensor that automatically collects data to detect probable eating events; an imaging member that is used by the person to take pictures of food wherein the person is prompted to take pictures of food when an eating event is detected by the wearable sensor; and a data analysis component that analyzes these food pictures to estimate the types and amounts of foods, ingredients, nutrients, and/or calories that are consumed by the person. U.S. patent application 2014034925 by Connor entitled “Smart Watch and Food Utensil for Monitoring Food Consumption” discloses a device and system for monitoring a person's food consumption comprising: a wearable sensor that automatically collects data to detect eating events; a smart food utensil, probe, or dish that collects data concerning the chemical composition of food which the person is prompted to use when an eating event is detected; and a data analysis component that analyzes chemical composition data to estimate the types and amounts of foods, ingredients, nutrients, and/or calories consumed by the person.

U.S. patent application 20150126873 by Connor entitled “Wearable Spectroscopy Sensor to Measure Food Consumption” discloses a wearable device to measure a person's consumption of selected types of food, ingredients, or nutrients comprising: a housing that is configured to be worn on the person's wrist, arm, hand, or finger; a spectroscopy sensor that collects data concerning light energy reflected from the person's body and/or absorbed by the person's body, wherein this data is used to measure the person's consumption of selected types of food, ingredients, or nutrients; a data processing unit; and a power source. U.S. patent application 20150168365 by Connor entitled “Caloric Intake Measuring System Using Spectroscopic and 3D Imaging Analysis” discloses a caloric intake measuring system comprising: a spectroscopic sensor that collects data concerning light that is absorbed by or reflected from food, wherein this food is to be consumed by a person, and wherein this data is used to estimate the composition of this food; and an imaging device that takes images of this food from different angles, wherein these images from different angles are used to estimate the quantity of this food.

Application WO 2010/070645 by Einav et al. entitled “Method and System for Monitoring Eating Habits” discloses an apparatus for monitoring eating patterns which can include a spectrometer for detecting nutritious properties of a bite of food. U.S. Pat. No. 8,355,875 by Hyde et al. entitled “Food Content Detector” discloses a utensil, which can include a spectroscopy sensor, for portioning a foodstuff into first and second portions. U.S. Pat. No. 8,355,875 by Hyde et al. entitled “Food Content Detector” discloses a utensil for portioning a foodstuff into first and second portions which can include a spectroscopy sensor. U.S. patent application 20150302160 by Muthukumar et al. entitled “Method and Apparatus for Monitoring Diet and Activity” discloses a method and apparatus including a camera and spectroscopy module for determining food types and amounts. U.S. patent 20140320858 by Goldring et al. is entitled “Low-Cost Spectrometry System for End-User Food Analysis.” This patent appears to be associated with SCiO—a molecular sensor which appears to use near-infrared spectroscopy to analyze the composition of nearby objects and may be used to analyze the composition of food. DietSensor™ is a company which has integrated the SCiO molecular sensor into a mobile phone compatible application.

The AIRO wristband raised money on Kickstarter in 2013. It was generally described in an article entitled “Wearable Tech Company Revolutionizes Health Monitoring” by Nicole Fallon in Business News Daily on Oct. 29, 2013. This article generally describes the wristband as “using light wavelengths to monitor nutrition, exercise, stress and sleep patterns,” but does provide many details on device structure or function. A search did not show any related patent applications. The company appears to have subsequently refunded money contributed to it by crowd-funding supporters.

The TellSpec™ hand-held sensor raised money on Indiegogo in 2014. It appears to have been intended as a hand-held device which uses spectroscopy to measure the nutrient composition of food. The company does not appear to have launched the device yet. Their U.S. patent application 20150036138 by Watson et al. entitled “Analyzing and Correlating Spectra, Identifying Samples and Their Ingredients, and Displaying Related Personalized Information” describes obtaining two spectra from the same sample under two different conditions at about the same time for comparison. The Healbe GoBe™ raised money on Indiegogo in 2014. It appears to be a wristband that is intended to measure caloric intake using electromagnetic energy. Thus far, there do not yet appear to be results published in a peer-reviewed journal. The BioRing™ launched a campaign on Indiegogo in 2016. It appears to be a ring that is intended to measure caloric intake using a biosensor.

### SUMMARY OF THE INVENTION

This invention is finger-worn device for monitoring a person's food consumption comprising: a finger ring; an electromagnetic energy emitter which emits electromagnetic energy into a person's finger tissue at a first location; an electromagnetic energy receiver which receives electromagnetic energy from the person's finger tissue at a second location, wherein parameters or patterns of the received electromagnetic energy are changed by the person's consumption of food and analyzed to monitor the person's food consumption; a power source; a data processor; and a data transmitter.

In an example, this device can monitor a person's food consumption by measuring changes in the electromagnetic impedance, resistance, conductivity, or permittivity of finger tissue. In an example, this finger-worn device can further comprise an electromagnetic resonator between an electromagnetic energy emitter and an electromagnetic energy receiver. In an example, an electromagnetic resonator can comprise a split ring. In an example, an electromagnetic resonator can comprise two or more nested rings. In an example, an electromagnetic resonator can comprise two or more stacked rings. In an example, an electromagnetic resonator can comprise a spiral.

### INTRODUCTION TO THE FIGURES

FIGS. 1 and 2 show a finger ring for analyzing the composition of food.

FIG. 3 shows a device with biometric sensors at different locations on the device circumference.

FIG. 4 shows a device with biometric sensors with different light-projection angles.

FIG. 5 shows a device with a rotating biometric sensor.

FIG. 6 shows a device with a two-dimensional array of biometric sensors.

FIG. 7 shows a device with biometric sensors pushed inward by hydraulic, pneumatic, or electromagnetic mechanisms.

FIG. 8 shows a device with biometric sensors pushed inward by individual springs.

FIG. 9 shows a device with biometric sensors on an inward-facing surface connected to an elastic compartment.

FIG. 10 shows a device with biometric sensors on an inward-facing surface that pivots around a joint.

FIG. 11 shows a device with biometric sensors on an inward-facing surface pressed inward by springs.

FIG. 12 shows a device with a biometric sensor on an elastic compartment.

FIG. 13 shows a device with multiple biometric sensors on an elastic compartment.

FIG. 14 shows a device with a biometric sensor on an elastic compartment with adjustable pressure.

FIG. 15 shows a device with biometric sensors on elastic compartments on a strap or band.

FIG. 16 shows a device with biometric sensors on toroidal elastic compartments.

FIG. 17 shows a device with biometric sensors on interconnected toroidal elastic compartments.

FIG. 18 shows a device with biometric sensors on inner elastic compartments connected to outer elastic compartments.

FIG. 19 shows a device with biometric sensors which can adjustably slide around the circumference of the device.

FIG. 20 shows a device with a biometric sensor on a laterally-rotating enclosure.

FIG. 21 shows a device with a biometric sensor on a threaded rotating enclosure.

FIG. 22 shows a device with a two-way location-adjustable biometric sensor.

FIG. 23 shows a device with two parallel bands and a biometric sensor that adjustably slides between them.

FIG. 24 shows a device with a biometric sensor on a rotating ball.

FIG. 25 shows a device with an undulating band with biometric sensors.

FIG. 26 shows a device with an undulating band with six undulations and biometric sensors.

FIG. 27 shows a device with a laterally-undulating band with biometric sensors.

FIG. 28 shows a device with one or more elastic portions which are configured to span the anterior (upper) surface of a person's arm, one or more inelastic portions which are configured to span the posterior (lower) surface of the person's arm, an enclosure which is connected to the elastic portions, and one or more biometric sensors which are part of the enclosure.

FIG. 29 shows a device with one or more anterior inelastic portions which are configured to span the anterior (upper) surface of a person's arm, one or more posterior inelastic portions which are configured to span the posterior (lower) surface of a person's arm, one or more elastic portions which connect the anterior and posterior inelastic portions, an enclosure which is configured to be worn on the anterior (upper) portion of the arm, and one or more biometric sensors which are part of the enclosure.

FIG. 30 shows a device with one or more biometric sensors in an enclosure and an attachment member which holds the enclosure on a person's arm, wherein there are rectangular, rounded rectangular, or plano-concave elastic portions of the attachment member which are connected to the enclosure and wherein the rest of the attachment member is inelastic.

FIG. 31 shows a device with elastic portions which are tapered as they approach an enclosure.

FIG. 32 shows a device with four elastic portions, two connected to each side of an enclosure.

FIG. 33 shows a device with two elastic portions on each side of an enclosure which criss-cross each other, forming an “X” on each side of the enclosure.

FIG. 34 shows a device with two parallel elastic bands (or straps) connected to an enclosure.

FIG. 35 shows a device with three parallel elastic bands (or straps) connected to an enclosure.

FIG. 36 shows a device wherein three elastic bands (or straps) connected to an enclosure are not parallel.

FIG. 37 shows a device with biometric sensors in an enclosure, wherein the enclosure is suspended on the surface of the arm by four elastic bands, and wherein each elastic band is individually connected to one of four points which are equally-spaced around the circumference of the enclosure.

FIG. 38 shows a device with biometric sensors in an enclosure, wherein the enclosure is suspended on the surface of the arm by four elastic suspension bands (or straps) connected to three parallel attachment bands or straps which encircle the arm.

FIG. 39 shows a device with a gimbaled enclosure with one or more biometric sensors.

FIG. 40 shows a device with an enclosure containing one or more biometric sensors, wherein this enclosure is suspended by a radial plurality of elastic (and/or stretchable or springy) suspension members which connect to locations on the circumference of the enclosure.

FIG. 41 shows a device with two arcuate enclosures which contain biometric sensors.

FIG. 42 shows a device with an arcuate enclosure to which a strap or band is connected diagonally

FIG. 43 shows a device that looks like two “gummi worms” crawling in a symmetric manner around portions of the circumference of an enclosure.

FIG. 44 shows a device with a bifurcating band, wherein one branch of the band is connected to a display screen and the other branch of the band is connected to a circumferentially-sliding enclosure which contains one or more biometric sensors.

FIG. 45 shows a device with a plurality of various-shaped (rigid) polygons which are inter-connected by flexible strips and/or joints, an enclosure which is connected to the attachment member, and one or more biometric sensors which are part of the enclosure.

FIG. 46 shows a device with a honeycomb configuration.

FIG. 47 shows a device comprising: an attachment member which spans at least 60% of the circumference of a person's arm, wherein this attachment member further comprises a first portion with a first elasticity level spanning completely or partially (clockwise) between the 9 o'clock and 3 o'clock (or 270-degree and 90-degree) positions around the device circumference and a second portion with a second elasticity level spanning completely or partially (clockwise) between the 3 o'clock and 9 o'clock (or 90-degree and 270-degree) positions around the device circumference, wherein the second elasticity level is greater than the first elasticity level; a display screen which is part of (or connected to) the first portion of the attachment member; an enclosure which is part of (or connected to) the second portion of the attachment member; and one or more biometric sensors which are configured to collect data concerning arm tissue and which are part of (or attached to) the enclosure.

FIG. 48 shows a device with one or more biometric sensors which are located on a portion of the device which is diametrically-opposite from the portion of the device which includes a display screen and there is a connector on the device between the sensors and the screen.

FIG. 49 shows a device with a “clam shell” design.

FIG. 50 shows a device with a “clam shell” design and a biometric sensor on the center-facing surface of a compressible member.

FIG. 51 shows a device with proximal and distal arcuate display screens which are attached to a person's arm by a band, wherein the band has one hole on each side of a virtual line connecting the centers of the two displays.

FIG. 52 shows a device with: proximal and distal arcuate display screens; and right and left side enclosures with biometric sensors.

FIG. 53 shows a device with proximal and distal arcuate display screens which are circumferentially attached to an arm by a bifurcating band (or strap) and also connected to each other by a band (or strap) along the central longitudinal axis of the anterior (upper) surface of the arm.

FIG. 54 shows a device with proximal and distal arcuate display screens which are attached to a person's arm by a band with an “S”-shaped portion spanning the anterior (upper) portion of the arm.

FIG. 55 shows a device with two bands which encircle an arm, wherein these two bands are movably-attached to each other in a manner which allows a second band (with biometric sensors) to be rotated relative to a first band.

FIG. 56 shows a device with three bands and biometric sensors on a central band which rotates relative to distal and proximal bands.

FIG. 57 shows a device with a relatively-rigid band and a relatively-elastic band, wherein each of these bands spans at least 60% of the circumference of a person's arm, wherein these bands are connected to each other, and wherein there are biometric sensors on the relatively-elastic band.

FIG. 58 shows a device with two or more modular and connectable bands, wherein each band spans at least 60% of the circumference of a person's arm, and wherein one or more of these bands house biometric sensors.

FIG. 59 shows a device with a partial-circumferential inner elastic band and biometric sensors.

FIG. 60 shows a device wherein an outer inelastic band is sufficiently resilient that its ends hold onto the person's arm without the need for a clasp.

FIG. 61 shows a device with an outer arcuate inelastic band, an inner arcuate elastic band, and biometric sensors which are part of the inner band.

FIG. 62 shows a device with an outer rigid “clam shell” structure to hold a display screen in place and an inner arcuate elastic band to keep biometric sensors close against the surface of the arm.

FIG. 63 shows a device with an inner arcuate elastic band which spans the posterior (lower) surface of a person's arm.

FIG. 64 shows a device with an outer rigid “clam shell” structure and inward-facing flexible undulations to keep biometric sensors close against the surface of the arm.

FIG. 65 shows a device with two display screens suspended by an elastic material between two arcuate bands.

FIG. 66 shows a device with two display screens which are suspended by elastic straps between two arcuate bands.

FIG. 67 shows a device with two display screens whose centers are at 12 o'clock and 6 o'clock positions around a circular band on the anterior (upper) surface of an arm.

FIG. 68 shows a device with two display screens suspended by an oval (or elliptical or circular) elastic member between two arcuate bands.

FIG. 69 shows a glucose monitoring system with an electromagnetic microwave sensor.

FIG. 70 shows a glucose monitoring system with an electromagnetic microwave sensor and electromagnetic resonator.

FIGS. 71 and 72 show a finger ring with an electromagnetic energy sensor for monitoring food consumption.

FIGS. 73 and 74 show a finger ring with an electromagnetic energy sensor and electromagnetic resonator for monitoring food consumption.

### DETAILED DESCRIPTION OF THE FIGURES

A device, system, or method for measuring a person's consumption of at least one selected type of food, ingredient, and/or nutrient is not a panacea for good nutrition, energy balance, and weight management, but it can be a useful part of an overall strategy for encouraging good nutrition, energy balance, weight management, and health improvement. Although such a device, system, or method is not sufficient to ensure energy balance and good health, it can be very useful in combination with proper exercise and other good health behaviors. Such a device, system, or method can help a person to track and modify their eating habits as part of an overall system for good nutrition, energy balance, weight management, and health improvement.

In an example, at least one component of such a device can be worn on a person's body or clothing. A wearable food-consumption monitoring device or system can operate in a more-consistent manner than an entirely hand-held food-consumption monitoring device, while avoiding the potential invasiveness and expense of a food-consumption monitoring device that is implanted within the body.

Information from a food-consumption monitoring device that measures a person's consumption of at least one selected type of food, ingredient, and/or nutrient can be combined with information from a separate caloric expenditure monitoring device that measures a person's caloric expenditure to comprise an overall system for energy balance, fitness, weight management, and health improvement. In an example, a food-consumption monitoring device can be in wireless communication with a separate fitness monitoring device. In an example, capability for monitoring food consumption can be combined with capability for monitoring caloric expenditure within a single device. In an example, a single device can be used to measure the types and amounts of food, ingredients, and/or nutrients that a person consumes as well as the types and durations of the calorie-expending activities in which the person engages.

Information from a food-consumption monitoring device that measures a person's consumption of at least one selected type of food, ingredient, and/or nutrient can also be combined with a computer-to-human interface that provides feedback to encourage the person to eat healthy foods and to limit excess consumption of unhealthy foods. In an example, a food-consumption monitoring device can be in wireless communication with a separate feedback device that modifies the person's eating behavior. In an example, capability for monitoring food consumption can be combined with capability for providing behavior-modifying feedback within a single device. In an example, a single device can be used to measure the selected types and amounts of foods, ingredients, and/or nutrients that a person consumes and to provide visual, auditory, tactile, or other feedback to encourage the person to eat in a healthier manner.

A combined device and system for measuring and modifying caloric intake and caloric expenditure can be a useful part of an overall approach for good nutrition, energy balance, fitness, weight management, and good health. As part of such an overall system, a device that measures a person's consumption of at least one selected type of food, ingredient, and/or nutrient can play a key role in helping that person to achieve their goals with respect to proper nutrition, food consumption modification, energy balance, weight management, and good health outcomes.

In an example, a food-consumption monitor or food-identifying sensor can be an electromagnetic sensor. In an example, an electromagnetic sensor that detects food or nutrient consumption can detect electromagnetic signals from the body in response to the consumption or digestion of food. In an example, analysis of this electromagnetic energy can help to identify the types of food that a person consumes.

In various examples, a sensor to detect food consumption or identify consumption of a selected type of nutrient can be selected from the group consisting of: neuroelectrical sensor, action potential sensor, ECG sensor, EKG sensor, EEG sensor, EGG sensor, capacitance sensor, conductivity sensor, impedance sensor, galvanic skin response sensor, variable impedance sensor, variable resistance sensor, interferometer, magnetometer, RF sensor, electrophoretic sensor, optoelectronic sensor, piezoelectric sensor, and piezocapacitive sensor.

In an example, a food-consumption monitor or food-identifying sensor can be a high-energy sensor. In an example, a high-energy sensor can identify a selected type of food, ingredient, or nutrient based on the interaction of microwaves or x-rays with a portion of food. In various examples a high-energy sensor to detect food consumption or identify consumption of a selected type of nutrient can be selected from the group consisting of: a microwave sensor, a microwave spectrometer, and an x-ray detector.

In various examples, a food-consumption monitor or food-identifying sensor can be selected from the group consisting of: chemical sensor, biochemical sensor, amino acid sensor, chemiresistor, chemoreceptor, photochemical sensor, optical sensor, chromatography sensor, fiber optic sensor, infrared sensor, optoelectronic sensor, spectral analysis sensor, spectrophotometer, olfactory sensor, electronic nose, metal oxide semiconductor sensor, conducting polymer sensor, quartz crystal microbalance sensor, electromagnetic sensor, variable impedance sensor, variable resistance sensor, conductance sensor, neural impulse sensor, EEG sensor, EGG sensor, EMG sensor, interferometer, galvanic skin response sensor, cholesterol sensor, HDL sensor, LDL sensor, electrode, neuroelectrical sensor, neural action potential sensor, Micro Electrical Mechanical System (MEMS) sensor, laboratory-on-a-chip, or medichip, micronutrient sensor, osmolality sensor, protein-based sensor or reagent-based sensor, saturated fat sensor or trans fat sensor, action potential sensor, biological sensor, enzyme-based sensor, protein-based sensor, reagent-based sensor, camera, video camera, fixed focal-length camera, variable focal-length camera, pattern recognition sensor, microfluidic sensor, motion sensor, accelerometer, flow sensor, strain gauge, electrogoniometer, inclinometer, peristalsis sensor, multiple-analyte sensor array, an array of cross-reactive sensors, pH level sensor, sodium sensor, sonic energy sensor, microphone, sound-based chewing sensor, sound-based swallow sensor, ultrasonic sensor, sugar sensor, glucose sensor, temperature sensor, thermometer, and thermistor.

In an example, a sensor to monitor, detect, or sense food consumption or to identify consumption of a selected type of food, ingredient, or nutrient can be a wearable sensor that is worn by the person whose food consumption is monitored, detected, or sensed. In an example, a wearable food-consumption monitor or food-identifying sensor can be worn directly on a person's body. In an example a wearable food-consumption monitor or food-identifying sensor can be worn on, or incorporated into, a person's clothing

In various examples, a wearable sensor can be worn on a person in a location selected from the group consisting of: wrist, neck, finger, hand, head, ear, eyes, nose, teeth, mouth, torso, chest, waist, and leg. In various examples, a wearable sensor can be attached to a person or to a person's clothing by a means selected from the group consisting of: strap, clip, clamp, snap, pin, hook and eye fastener, magnet, and adhesive.

In various examples, a wearable sensor can be worn on a person in a manner like a clothing accessory or piece of jewelry selected from the group consisting of: wristwatch, wristphone, wristband, bracelet, cufflink, armband, armlet, and finger ring; necklace, neck chain, pendant, dog tags, locket, amulet, necklace phone, and medallion; eyewear, eyeglasses, spectacles, sunglasses, contact lens, goggles, monocle, and visor; clip, tie clip, pin, brooch, clothing button, and pin-type button; headband, hair pin, headphones, ear phones, hearing aid, earring; and dental appliance, palatal vault attachment, and nose ring.

In an example, a wearable device can prompt a person to collect information concerning food consumption using a smart phone application. In an example, a wearable device can automatically activate a smart phone or other portable electronic device to collect information concerning food consumption. In an example, a wearable device can automatically trigger a smart phone or other portable electronic device to start recording audio information using the smart phone's microphone when the wearable device detects that the person is probably eating. In an example, a wearable device can automatically trigger a smart phone or other portable electronic device to start recording visual information using the smart phone's camera when the wearable device detects that the person is probably eating.

In an example, a food-consumption monitor or food-identifying sensor can monitor a particular body member. In various examples, such a monitor or sensor can be selected from the group consisting of: a blood monitor (for example using a blood pressure monitor, a blood flow monitor, or a blood glucose monitor); a brain monitor (such as an electroencephalographic monitor); a heart monitor (such as electrocardiographic monitor, a heartbeat monitor, or a pulse rate monitor); a mouth function monitor (such as a chewing sensor, a biting sensor, a jaw motion sensor, a swallowing sensor, or a saliva composition sensor); a muscle function monitor (such as an electromyographic monitor or a muscle pressure sensor); a nerve monitor or neural monitor (such as a neural action potential monitor, a neural impulse monitor, or a neuroelectrical sensor); a respiration monitor (such as a breathing monitor, an oxygen consumption monitor, an oxygen saturation monitor, a tidal volume sensor, or a spirometry monitor); a skin sensor (such as a galvanic skin response monitor, a skin conductance sensor, or a skin impedance sensor); and a stomach monitor (such as an electrogastrographic monitor or a stomach motion monitor). In various examples, a sensor can monitor sonic energy or electromagnetic energy from selected portions of a person's gastrointestinal tract (ranging from the mouth to the intestines) or from nerves which innervate those portions. In an example, a monitor or sensor can monitor peristaltic motion or other movement of selected portions of a person's gastrointestinal tract.

In an example, a food-consumption monitor or food-identifying sensor can be incorporated into a smart watch or other device that is worn on a person's wrist. In an example, a food-consumption monitor or food-identifying sensor can be worn on, or attached to, other members of a person's body or to a person's clothing. In an example, a device for measuring a person's consumption of at least one selected type of food, ingredient, or nutrient can be worn on, or attached to, a person's body or clothing. In an example, a device can be worn on, or attached to, a part of a person's body that is selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

In an example, a device for measuring a person's consumption of at least one selected type of food, ingredient, or nutrient can be worn in a manner similar to a piece of jewelry or accessory. In various examples, a food consumption measuring device can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

In an example, a device for measuring a person's consumption of at least one selected type of food, ingredient, or nutrient can be attached to a person's body or clothing. In an example, a device to measure food consumption can be attached to a person's body or clothing using an attachment means selected from the group consisting of: band, strap, chain, hook and eye fabric, ring, adhesive, bracelet, buckle, button, clamp, clip, elastic band, eyewear, magnet, necklace, piercing, pin, string, suture, tensile member, wrist band, and zipper. In an example, a device can be incorporated into the creation of a specific article of clothing. In an example, a device to measure food consumption can be integrated into a specific article of clothing by a means selected from the group consisting of: adhesive, band, buckle, button, clip, elastic band, hook and eye fabric, magnet, pin, pocket, pouch, sewing, strap, tensile member, and zipper.

In an example, a wearable device for measuring a person's consumption of at least one selected type of food, ingredient, or nutrient can comprise one or more sensors selected from the group consisting of: motion sensor, accelerometer (single multiple axis), electrogoniometer, or strain gauge; optical sensor, miniature still picture camera, miniature video camera, miniature spectroscopy sensor; sound sensor, miniature microphone, speech recognition software, pulse sensor, ultrasound sensor; electromagnetic sensor, skin galvanic response (Galvanic Skin Response) sensor, EMG sensor, chewing sensor, swallowing sensor; temperature sensor, thermometer, infrared sensor; and chemical sensor, chemical sensor array, miniature spectroscopy sensor, glucose sensor, cholesterol sensor, or sodium sensor.

In an example, a device and system for measuring a person's consumption of at least one selected type of food, ingredient, or nutrient can be entirely wearable or include a wearable component. In an example, a wearable device or component can be worn directly on a person's body, can be worn on a person's clothing, or can be integrated into a specific article of clothing. In an example, a wearable device for measuring food consumption can be in wireless communication with an external device. In various examples, a wearable device for measuring food consumption can be in wireless communication with an external device selected from the group consisting of: a cell phone, an electronic tablet, electronically-functional eyewear, a home electronics portal, an internet portal, a laptop computer, a mobile phone, a remote computer, a remote control unit, a smart phone, a smart utensil, a television set, and a virtual menu system.

In an example, a wearable device for measuring a person's consumption of at least one selected type of food, ingredient, or nutrient can comprise multiple components selected from the group consisting of: Central Processing Unit (CPU) or microprocessor; food-consumption monitoring component (motion sensor, electromagnetic sensor, optical sensor, and/or chemical sensor); graphic display component (display screen and/or coherent light projection); human-to-computer communication component (speech recognition, touch screen, keypad or buttons, and/or gesture recognition); memory component (flash, RAM, or ROM); power source and/or power-transducing component; time keeping and display component; wireless data transmission and reception component; and strap or band.

In an example, a device, method, and system for measuring consumption of selected types of foods, ingredients, or nutrients can include a hand-held component in addition to a wearable component. In an example, a hand-held component can be linked or combined with a wearable component to form an integrated system for measuring a person's consumption of at least one selected type of food, ingredient, or nutrient. In an example, the combination and integration of a wearable member and a hand-held member can provide advantages that are not possible with either a wearable member alone or a hand-held member alone. In an example, a wearable member of such a system can be a food-consumption monitor. In an example, a hand-held member of such a system can be a food-identifying sensor.

In an example, a food-consumption monitoring and nutrient identifying system can include a hand-held component that is selected from the group consisting of: smart phone, mobile phone, cell phone, holophone, or application of such a phone; electronic tablet, other flat-surface mobile electronic device, Personal Digital Assistant (PDA), or laptop; digital camera; and smart eyewear, electronically-functional eyewear, or augmented reality eyewear. In an example, such a hand-held component can be in wireless communication with a wearable component of such a system. In an example, a device, method, or system for detecting food consumption or measuring consumption of a selected type of food, ingredient, or nutrient can include integration with a general-purpose mobile device that is used to collects data concerning food consumption. In an example, the hand-held component of such a system can be a general purpose device, of which collecting data for food identification is only one among many functions that it performs. In an example, a system for measuring a person's consumption of at least one selected type of food, ingredient, or nutrient can comprise: a wearable member that continually monitors for possible food consumption; a hand-held smart phone that is used to take pictures of food that will be consumed; wireless communication between the wearable member and the smart phone; and software that integrates the operation of the wearable member and the smart phone.

In various examples, a device for measuring a person's consumption of at least one selected type of food, ingredient, or nutrient can provide feedback to the person that is selected from the group consisting of: auditory feedback (such as a voice message, alarm, buzzer, ring tone, or song); feedback via computer-generated speech; mild external electric charge or neural stimulation; periodic feedback at a selected time of the day or week; phantom taste or smell; phone call; pre-recorded audio or video message by the person from an earlier time; television-based messages; and tactile, vibratory, or pressure-based feedback.

In various examples, a device for measuring a person's consumption of at least one selected type of food, ingredient, or nutrient can provide feedback to the person that is selected from the group consisting of: feedback concerning food consumption (such as types and amounts of foods, ingredients, and nutrients consumed, calories consumed, calories expended, and net energy balance during a period of time); information about good or bad ingredients in nearby food; information concerning financial incentives or penalties associated with acts of food consumption and achievement of health-related goals; information concerning progress toward meeting a weight, energy-balance, and/or other health-related goal; information concerning the calories or nutritional components of specific food items; and number of calories consumed per eating event or time period.

In various examples, a device for measuring a person's consumption of at least one selected type of food, ingredient, or nutrient can provide feedback to the person that is selected from the group consisting of: augmented reality feedback (such as virtual visual elements superimposed on foods within a person's field of vision); changes in a picture or image of a person reflecting the likely effects of a continued pattern of food consumption; display of a person's progress toward achieving energy balance, weight management, dietary, or other health-related goals; graphical display of foods, ingredients, or nutrients consumed relative to standard amounts (such as embodied in pie charts, bar charts, percentages, color spectrums, icons, emoticons, animations, and morphed images); graphical representations of food items; graphical representations of the effects of eating particular foods; holographic display; information on a computer display screen (such as a graphical user interface); lights, pictures, images, or other optical feedback; touch screen display; and visual feedback through electronically-functional eyewear.

In various examples, a device for measuring a person's consumption of at least one selected type of food, ingredient, or nutrient can provide feedback to the person that is selected from the group consisting of: advice concerning consumption of specific foods or suggested food alternatives (such as advice from a dietician, nutritionist, nurse, physician, health coach, other health care professional, virtual agent, or health plan); electronic verbal or written feedback (such as phone calls, electronic verbal messages, or electronic text messages); live communication from a health care professional; questions to the person that are directed toward better measurement or modification of food consumption; real-time advice concerning whether to eat specific foods and suggestions for alternatives if foods are not healthy; social feedback (such as encouragement or admonitions from friends and/or a social network); suggestions for meal planning and food consumption for an upcoming day; and suggestions for physical activity and caloric expenditure to achieve desired energy balance outcomes.

In various examples, a device and system for measuring food consumption can be in wireless communication with an external device or system selected from the group consisting of: internet portal; smart phone, mobile phone, cell phone, holophone, or application of such a phone; electronic tablet, other flat-surface mobile electronic device, Personal Digital Assistant (PDA), remote control unit, or laptop; smart eyewear, electronically-functional eyewear, or augmented reality eyewear; electronic store display, electronic restaurant menu, or vending machine; and desktop computer, television, or mainframe computer. In various examples, a device can receive food-identifying information from a source selected from the group consisting of: electromagnetic transmissions from a food display or RFID food tag in a grocery store, electromagnetic transmissions from a physical menu or virtual user interface at a restaurant, and electromagnetic transmissions from a vending machine.

In an example, a wearable device and system for measuring a person's consumption of at least one selected type of food, ingredient, or nutrient can be tamper resistant. In an example, a wearable device can detect when it has been removed from the person's body by monitoring signals from the body such as pulse, motion, heat, skin electromagnetism, or proximity to an implanted device. In an example, a wearable device for measuring food consumption can detect if it has been removed from the person's body by detecting a lack of motion, lack of a pulse, and/or lack of electromagnetic response from skin. In various examples, a wearable device for measuring food consumption can continually monitor optical, electromagnetic, temperature, pressure, or motion signals that indicate that the device is properly worn by a person. In an example, a wearable device can trigger feedback if the device is removed from the person and the signals stop.

In an example, an optical sensor can be configured to have a sensing direction which points outward from the surface of a person's body or clothing. In an example, an optical sensor can be a spectroscopic optical sensor. In an example, a spectroscopic sensor can be a part of a wearable device which is configured to be worn on a person's finger. In an example, a spectroscopic sensor can be a part of an electronically-functional ring. A wearable sensor can be worn on a person in a manner like a finger ring. In an example, a spectroscopic sensor can collect data concerning the spectrum of light that is transmitted through and/or reflected from nearby food. In an example, a sensor can be selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

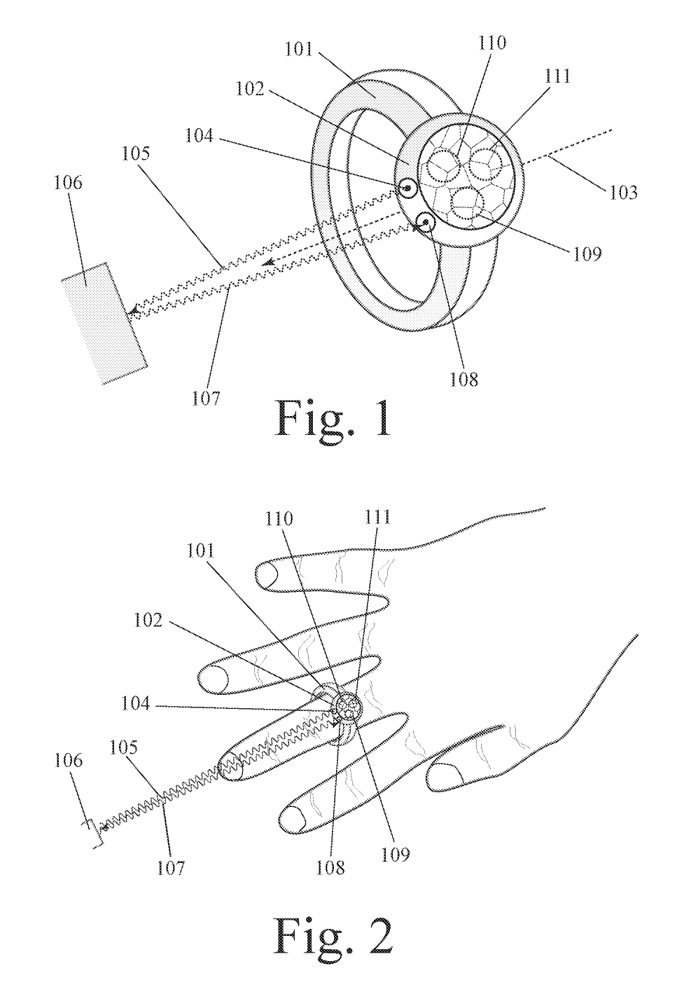

FIGS. 1 and 2 show an example of a spectroscopic finger ring for compositional analysis of food or some other environmental object. This spectroscopic finger ring is one embodiment of a wearable device configured worn on a person's hand including a spectroscopic optical sensor that collects data concerning the spectrum of light that is reflected from (or has passed through) nearby food or some other environmental object. This light spectrum data is analyzed in order to estimate the chemical composition of the food or other environmental object. FIG. 1 shows a close-up view of this finger ring before it is worn. FIG. 2 shows an overall view of this same finger as it is worn on a person's hand

The example shown in FIGS. 1 and 2 is a spectroscopic finger ring for compositional analysis of environmental objects comprising: a ring which is configured to be worn on a person's finger, wherein this ring further comprises a light-emitting member which projects a beam of light away from the person's body toward food or some other environmental object, and wherein this ring further comprises a spectroscopic optical sensor which collects data concerning the spectrum of light which is reflected from (or has passed through) the food or other environmental object.

Looking at this example in more detail, FIGS. 1 and 2 show: a finger-encircling portion 101 of a finger ring; an anterior (or upper) portion 102 of the finger ring; a central proximal-to-distal axis 103 of the finger ring; a light-emitting member 104; an outward-directed light beam 105; a piece of food or other environmental object 106; an inward-directed light beam 107; a spectroscopic optical sensor 108; a data processing unit 109; a power source 110; and a data transmitting unit 111.

In an example, a finger-encircling portion of a ring can have a shape which is selected from the group consisting of: circle, ellipse, oval, cylinder, torus, and volume formed by three-dimensional revolution of a semi-circle. In an example, a finger-encircling portion of a ring can be made from a metal or polymer. In an example, a finger-encircling portion of a ring can have a proximal-to-distal width between ⅛″ to 2″. In an example, proximal can be defined as closer to a person's elbow (or further from a finger tip) and distal can be defined as further from a person's elbow (or closer to a finger tip).

In an example, an anterior (or upper) portion of a finger ring can be made separately and then attached to the finger-encircling portion of the ring. In an example, an anterior (or upper) portion of a finger ring can be an integral portion of the finger-encircling portion of the ring which widens, thickens, bulges, spreads, and/or bifurcates as it spans the anterior (or upper) surface of a finger. In an example, an anterior (or upper) portion of a finger ring can have a cross-sectional shape which is selected from the group consisting of: circle, ellipse, oval, egg shape, tear drop, hexagon, octagon, quadrilateral, and rounded quadrilateral. In an example, an anterior (or upper) portion of a finger ring can be ornamental. In an example, an anterior (or upper) portion of a finger ring can be a gemstone or at least look like a gemstone. In an example, an anterior (or upper) portion of a finger ring can include a display screen. In an example, the anterior (or upper) portion of a finger ring can rotate.

In an example, a central proximal-to-distal axis of a finger ring can be defined as the straight line which most closely fits a proximal-to-distal series of centroids of interior cross-sectional perimeters of the finger-encircling portion of the finger ring. If the shape of a finger ring is approximated by a cylinder or torus, then its central proximal-to-distal axis connects the centers of cross-sectional circles comprising the cylinder or torus. In an example, a finger proximal-to-distal axis can be defined as the central longitudinal axis of a phalange on which a finger ring is configured to be worn. If the shape of a phalange is approximated by a cylinder, then its central proximal-to-distal axis connects the centers of cross-sectional circles comprising the cylinder.

In an example, a light-emitting member can be an LED (Light Emitting Diode). In an example, a light-emitting member can be a laser. In an example, a spectroscopic finger ring can have two or more light-emitting members instead of just one. In an example, a light-emitting member can emit an outward-directed beam of light away from the surface of a person's body. In an example, an outward-directed beam of light from a light-emitting member can comprise near-infrared light. In an example, an outward-directed beam of light from a light-emitting member can comprise infrared light. In an example, an outward-directed beam of light from a light-emitting member can comprise ultra-violet light. In an example, an outward-directed beam of light from a light-emitting member can comprise white light. In an example, an outward-direction beam of light from a light-emitting member can comprise coherent light. In an example, an outward-direction beam of light from a light-emitting member can comprise polarized light.

In an example, a light-emitting member can be part of (or attached to) the anterior (or upper) portion of a finger ring. In an example, a spectroscopic optical sensor in a finger ring can have an outward projection vector which points away from a person's body and toward food or some other environmental object. In an example, a light-emitting member can emit an outward-directed beam of light from the distal portion of the anterior (or upper) portion of a finger ring. In an example, a light-emitting member can emit an outward-directed beam of light in a proximal-to-distal direction. In an example, when a person points their finger at food or some other environmental object, then this outward-directed beam is directed toward that food or other environmental object. In an example, when a person grasps food or some other environmental object with their hand, then this outward-directed beam is directed toward that food or other environmental object.

In an example, a light-emitting member can emit an outward-directed beam of light in a proximal-to-distal vector which is substantially parallel to the central proximal-to-distal axis of a finger ring. In an example, a light-emitting member can emit an outward-directed beam of light in a proximal-to-distal vector which is substantially parallel to the proximal-to-distal axis of the phalange on which a ring is worn. In an example, a light-emitting member can emit an outward-directed beam of light along a vector which intersects (or whose virtual forward or backward extension intersects) a line which is parallel to the central proximal-to-distal axis of the finger ring. In an example, this intersection forms a distal-opening (or proximal-pointing) angle theta. In an example, the absolute value of theta is less than 20 degrees. In an example, the absolute value of theta is less than 45 degrees. In an example, a light-emitting member can emit an outward-directed beam of light along a vector which intersects (or whose virtual forward or backward extension intersects) a line which is parallel to the central proximal-to-distal axis of the phalange on which the ring is worn. In an example, this intersection forms a distal-opening (or proximal-pointing) angle theta. In an example, the absolute value of theta is less than 20 degrees. In an example, the absolute value of theta is less than 45 degrees.

In an example, the vector direction of an outward-directed beam of light emitted by a light-emitting member can be changed by the person wearing the finger ring. In an example, this vector can be automatically changed by the device in response to (changes in) the location of food or some other environmental object. In an example, this vector can be automatically moved in an iterative manner in order to automatically scan for food or some other environmental object. In an example, this vector can be automatically moved in an iterative manner in order to automatically scan a large portion of the surface of food or some other environmental object. In an example, the vector direction of an outward-directed beam of light can be changed by rotating the anterior (or upper) portion of a finger ring. In an example, the vector direction of an outward-directed beam of light can be changed by moving a mirror inside the anterior (or upper) portion of a finger ring.

In an example, a spectroscopic optical sensor can receive inward-directed light which has been reflected from (or passed through) food or some other environmental object. In an example, the reflection of light from the surface of the food or some other environmental object changes the spectrum of light which is then measured by the spectroscopic optical sensor in order to estimate the chemical composition of the food or other environmental object. In an example, the passing of light through food or some other environmental object changes the spectrum of light which is then measured by the spectroscopic optical sensor in order to estimate the chemical composition of the food or other environmental object. In an example, inward-directed light can originate with the outward-directed beam of light from the light-emitting member. In an example, inward-directed light can originate from an ambient light source.

In an example, data from a spectroscopic optical sensor can be analyzed in order to estimate the chemical composition of food or some other environmental object. In an example, data from a spectroscopic optical sensor can be analyzed in order to measure the composition of an environmental object from which an outward-directed beam of light has been reflected. In an example, a spectroscopic optical sensor can be selected from the group consisting of: spectrometry sensor; white light and/or ambient light spectroscopic sensor; infrared spectroscopic sensor; near-infrared spectroscopic sensor; ultraviolet spectroscopic sensor; ion mobility spectroscopic sensor; mass spectrometry sensor; backscattering spectrometric sensor; and spectrophotometer.

In an example, a light-emitting member and a spectroscopic optical sensor can share the same opening, compartment, or location in a finger ring. In an example, a light-emitting member and a spectroscopic optical sensor can be aligned along the same proximal-to-distal axis. In an example, an outward-directed beam of light emitted by a light-emitting member can be substantially parallel to (and even coaxial with) an inward-directed beam of light received by a spectroscopic optical sensor. In an example, a light-emitting member and a spectroscopic optical sensor can occupy different openings, compartments, or locations on a finger ring. In an example, an outward-directed beam of light emitted by a light-emitting member and an inward-directed beam of light received by a spectroscopic optical sensor can travel at different angles along non-parallel vectors.

In an example, the vector along which an outward-directed beam of light is emitted can be selected in order to direct reflected light back to the spectroscopic optical sensor from an object at a selected focal distance. In an example, this selected focal distance can be selected manually by the person wearing the ring. In an example, this selected focal distance can be selected based on detection of food or some other environmental object at a selected distance from the ring. In an example, detection of food or some other environmental object (and its distance) can be based on image analysis, reflection of light energy, reflection of radio waves, reflection of sonic energy, or gesture recognition. In an example, the vector along which an outward-directed beam of light is emitted can be varied in order to scan across different distances (or focal depths) in the surrounding environment.

In an alternative example, a spectroscopic finger ring can have an optical spectroscopic sensor, but no light-emitting member. In such an example, an optical spectroscopic sensor can receive ambient light which has been reflected from (or passed through) food or some other environmental object. In an alternative example, a spectroscopic finger ring can have a member which reflects and/or redirects ambient light toward food or some other environmental object instead of using a light-emitting member. In such an example, a spectroscopic finger ring can have a minor or lens which is adjusted in order to direct sunlight (or other ambient light) toward food or some other environmental object. In an example, the reflection of this ambient light from the food or other environmental object can be analyzed in order to estimate the chemical composition of the food or other environmental object.

In an example, a finger ring device can further comprise a motion sensor. In an example, a finger ring device can further comprise an accelerometer and/or gyroscope. In an example, motion patterns can be analyzed to determine optimal times for initiating a spectroscopic scan of food or some other environmental object. In an example, motion patterns can be analyzed to identify eating patterns. In an example, spectroscopic scans can be triggered at times during eating when a person's arm is most extended and, thus, most likely to be closest to the remaining uneaten portion of food. In an example, a spectroscopic scan can be triggered by a gesture indicating that a person is grasping food or bringing food up to their mouth. In an example, repeated spectroscopic scans of food at different times during a meal can help to analyze the composition of multiple food layers, not just the surface layer. This can provide a more accurate estimate of food composition, especially for foods with different internal layers and/or a composite (non-uniform) ingredient structure.

In an example, a finger ring device can further comprise a visible laser beam. In an example, this visible laser beam can be separate from the outward-directed beam of light that is used for spectroscopic analysis. In an example, a visible laser beam can be used by the person in order to point the spectroscopic beam toward food or some other environmental object for compositional analysis. In an example, a person can “point and click” by pointing the laser beam toward an object and then tapping, clicking, or pressing a portion of the finger ring in order to initiate a spectroscopic scan of the object. In an example, a person can point the laser beam toward the object and then give a verbal command to initiate a spectroscopic scan of the object. In an example, a finger ring device can further comprise a camera which takes a picture of the food or other environmental object. In an example, spectroscopic analysis can reveal the composition of the food (or object) and analysis of images from the camera can estimate the size of the food (or object). In an example, a visible laser beam can serve as a fiducial marker for image analysis.

In an example, a spectroscopic finger ring can be controlled by gesture recognition. In an example, a spectroscopic finger ring can be triggered by pointing at food or some other environmental object. In an example, a spectroscopic finger ring can be controlled by making a specific hand gesture. In an example, a spectroscopic finger ring can be directed to scan the entire surface of nearby food or some other environmental object by a hand gesture.

In an example, a spectroscopic finger ring can be worn on the proximal phalange of a person's finger, in a manner like a conventional ring. In an example, a spectroscopic finger ring can be worn on the middle or distal phalange of a person's finger in order to be more accurately directed toward an object held between the fingers, grasped by the hand, or pointed at by the person. In an example, a spectroscopic finger ring can be worn on a person's ring finger, in a manner like a conventional ring. In an example, a spectroscopic finger ring can be worn on a person's index finger in order to be more accurately directed toward an object held between the person's fingers, grasped by the person's hand, or pointed at by the person. In an example, a spectroscopic finger ring can be worn on a person's middle finger or pinky. In an example, joint analysis of data from a plurality of spectroscopic finger rings can provide more accurate information than data from a single spectroscopic finger ring. In an example, a plurality of spectroscopic finger rings can be worn on the proximal, middle, and/or distal phalanges of a person's finger. In an example, a plurality of spectroscopic finger rings can be worn on a person's index, middle, ring, and/or pinky fingers.

In an example, a finger ring device can further comprise a local data processing unit. In an example, data from an optical spectroscopic sensor can be at least partially processed by this local data processing unit. In an example, this data can be wirelessly transmitted to a remote data processing unit for further processing. In an example, this finger ring device can further comprise a data transmitting unit which wirelessly transmits data to another device and/or system component. In an example, the spectrum of light which has been reflected from (or passed through) food or some other environmental object can be used to help identify the chemical composition of that food or other environmental object. In an example, a change in the spectrum of outward-directed light from a light-emitting member vs. the spectrum of inward-directed light which has been reflected from (or passed through) food or some other environmental object can be used to help identify the chemical composition of that food or other environmental object.

In an example, a spectroscopic finger ring can be in wireless electromagnetic communication with a remote device. In an example, this remote device can be worn elsewhere on the person's body. In an example, a spectroscopic finger ring can be in electromagnetic communication with a smart watch or other wrist-worn device. In an example, information concerning the chemical composition of food or some other environmental object can be displayed on a smart watch or other wrist-worn device. In an example, a spectroscopic finger ring can be in electromagnetic communication with electronically-functional and/or augmented reality eyewear. In an example, information concerning the chemical composition of food or some other environmental object can be displayed via electronically-functional and/or augmented reality eyewear. In an example, a spectroscopic finger ring can be in wireless electromagnetic communication with a hand held device such as a cell phone. In an example, information concerning the chemical composition of food or some other environmental object can be displayed on a cell phone or other hand held electronic device.

In an example, information concerning the composition of food or some other environmental object based on data from a spectroscopic finger ring can be communicated in an auditory manner. In an example, this information can be communicated by voice from a wrist-worn device, electronically-functional eyewear, electronically-functional earwear, or a hand-held electronic device. For example, a person can point at an energy bar which is labeled “100% natural” and electronically-functional earwear can whisper into the person's ear—“Yeah, right . . . 50% natural sugar, 40% natural corn syrup, and 10% natural caffeine. They can call it natural, but it is not good nutrition.”

In an example, this finger ring device can further comprise a power source such as a battery and/or and energy-harvesting unit. In an example, an energy-harvesting unit can harvest energy from body motion, body temperature, ambient light, and/or ambient electromagnetic energy. In various examples, other relevant components and features discussed with respect to other examples in this disclosure can also be applied to the example shown in FIGS. 1 and 2.

In an example, a wearable spectroscopic sensor to measure food consumption based on the interaction between light and the human body can comprise a wearable food-consumption monitor that is configured to be worn on a person's wrist, arm, hand or finger. In an example, a wearable spectroscopic sensor to measure food consumption based on the interaction between light and the human body can monitor light energy that is reflected from a person's body tissue, absorbed by the person's body tissue, or has passed through the person's body tissue. In an example, a wearable spectroscopic sensor to measure food consumption based on the interaction between light and the human body can identify consumption of a selected type of food, ingredient, or nutrient using spectral analysis. In an example, a spectroscopic sensor can be a white light spectroscopic sensor, an infrared spectroscopic sensor, a near-infrared spectroscopic sensor, an ultraviolet spectroscopic sensor, an ion mobility spectroscopic sensor, a mass spectrometry sensor, a backscattering spectrometry sensor, or a spectrophotometer.

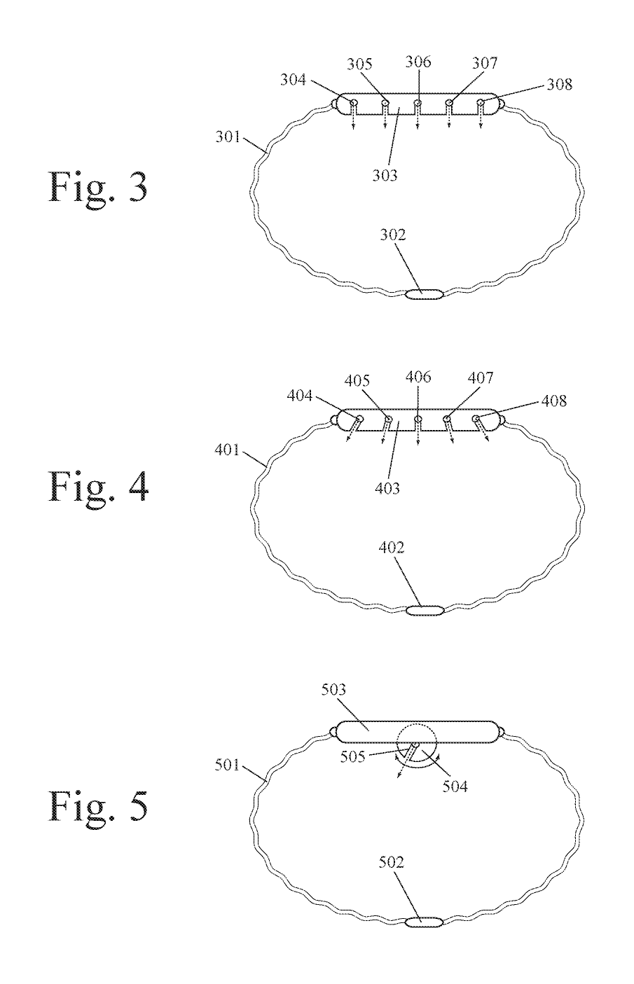

FIG. 3 shows an example of a wearable device for the arm with a plurality of close-fitting biometric sensors. This figure shows the device from a side perspective, as it would appear encircling a lateral cross-section of a person's wrist (or other portion of the person's arm). Described generally, the example shown in FIG. 3 is an arcuate wrist-worn device with a circumferentially-distributed array of biometric sensors. A series of circumference-center-facing biometric sensors are distributed along different locations on a portion of the circumference of the device. In this example, the array of sensors is distributed along the circumference-center-facing surface of an enclosure which is on the anterior (upper) portion of the device. In another example, an array of sensors can be distributed along the circumference-center-facing surface of a band or strap.

Having a circumferentially-distributed array of sensors allows a wearable device to record biometric measurements from different locations along the circumference of a person's wrist. This can help to find the best location on a person's wrist from which to most-accurately record biometric measurements. Having a circumferentially-distributed array of sensors can also enable a device to record biometric measurements from substantially the same location on a person's wrist, even if the device is unintentionally slid, shifted, and/or partially-rotated around the person's wrist. A different primary sensor can selected to record data when the device slides, shifts, and/or rotates. This can help to reduce biometric measurement errors when the device is slid, shifted, and/or partially-rotated around a person's wrist.

More specifically, the example shown in FIG. 3 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; (c) a first biometric sensor at a first location in the enclosure which is configured to record biometric data concerning the person's arm tissue; and (d) a second biometric sensor at a second location in the enclosure which is configured to record biometric data concerning the person's arm tissue, wherein the distance along the circumference of the device from the first location to second location is at least a quarter inch.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, a plurality of sensors can be housed within a single enclosure. In another example, different sensors can be housed in different enclosures. In another example, sensors can be located along the circumference-center-facing surface of an attachment member. In an example, there can be a display screen on the outward-facing surface of an enclosure.

In an example, first and second biometric sensors can be spectroscopic sensors which are each configured to measure the spectrum of light energy reflected from (and/or absorbed by) tissue of the person's arm. In an example, first and second biometric sensors can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 3 includes: strap (or band) 301, strap (or band) connector 302, enclosure 303, and spectroscopic sensors 304, 305, 306, 307, and 308. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 4 shows another example of a wearable device for the arm with a plurality of close-fitting biometric sensors. This figure shows the device from a side perspective, as it would appear encircling a lateral cross-section of a person's wrist (or other portion of the person's arm).

The example shown in FIG. 4 is like the one shown in FIG. 3 except that different sensors in the array of sensors direct light energy onto the surface of an arm at different angles relative to an enclosure. Having an array of sensors which direct light energy onto the surface of the arm at different angles relative to an enclosure can enable a device to record biometric measurements with substantially the same angle of incidence, even if the enclosure is tilted with respect to the surface of the person's wrist. A different primary sensor with a different angle of light projection can be selected to record data when the enclosure is tilted. For example, when an enclosure is parallel to the surface of the person's wrist, then a sensor with a 90-degree light projection angle (relative to the enclosure) can be selected so that light is projected onto the surface of the arm in a perpendicular manner. However, when the enclosure is tilted at a 20-degree angle relative to the surface of the person's wrist, then a sensor with a 70-degree angle (relative to the enclosure) can be selected so that light is again projected onto the surface of the arm in a perpendicular manner.

The example shown in FIG. 4 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; (c) a first spectroscopic sensor in the enclosure which is configured to project a beam of light onto the arm surface at a first angle relative to the enclosure; and (d) a second spectroscopic sensor in the enclosure which is configured to project a beam of light onto the arm surface at a second angle relative to the enclosure, wherein the first angle differs from the second angle by at least 10 degrees.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, a plurality of sensors can be housed within a single enclosure. In another example, different sensors can be housed in different enclosures. In another example, sensors can be located along the circumference-center-facing surface of an attachment member. In an example, there can be a display screen on the outward-facing surface of an enclosure.

With respect to specific components, the example shown in FIG. 4 includes: strap (or band) 401, strap (or band) connector 402, enclosure 403, and spectroscopic sensors 404, 405, 406, 407, and 408. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 5 shows an example of a wearable device for the arm with a close-fitting biometric sensor. This figure shows the device from a side perspective, as it would appear encircling a lateral cross-section of a person's wrist (or other portion of the person's arm).

Described generally, the example shown in FIG. 5 is an arcuate wrist-worn device with a rotating light-projecting spectroscopic sensor, wherein rotation of this sensor changes the angle at which it projects light onto the surface of a person's arm. In this example, the rotating light-projecting spectroscopic sensor is on the circumference-center-facing surface of an enclosure which is on the anterior (upper) portion of the device. In another example, such a sensor can be on the circumference-center-facing surface of a band or strap.

Having a rotating light-projecting spectroscopic sensor can enable a device to record biometric measurements with substantially the same angle of incidence, even if an enclosure is tilted with respect to the surface of the person's wrist. For example, when the enclosure is parallel to the surface of the person's wrist, then the rotating sensor is automatically rotated to project light at a 90-degree angle (relative to the enclosure) so that light is projected onto the surface of the arm in a perpendicular manner. However, when the enclosure is tilted at a 20-degree angle relative to the surface of the person's wrist, then the rotating sensor is automatically rotated to project light at a 70-degree angle (relative to the enclosure) so that light is again projected onto the surface of the arm in a perpendicular manner.

The example shown in FIG. 5 is a wearable device for the arm with a close-fitting biometric sensor comprising: (a) an attachment member, such as a strap or band, which is configured to span at least a portion of the circumference of a person's arm; (b) an enclosure which is part of (or attached to) the attachment member; and (c) a rotating light-projecting spectroscopic sensor, wherein this sensor can be rotated relative to the enclosure and wherein rotation of this sensor relative to the enclosure changes the angle at which the sensor projects light onto the surface of a person's arm.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, there can be a display screen on the outward-facing surface of an enclosure.

With respect to specific components, the example shown in FIG. 5 includes: strap (or band) 501, strap (or band) connector 502, enclosure 503, rotating member 504, and light-projecting spectroscopic sensor 505. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

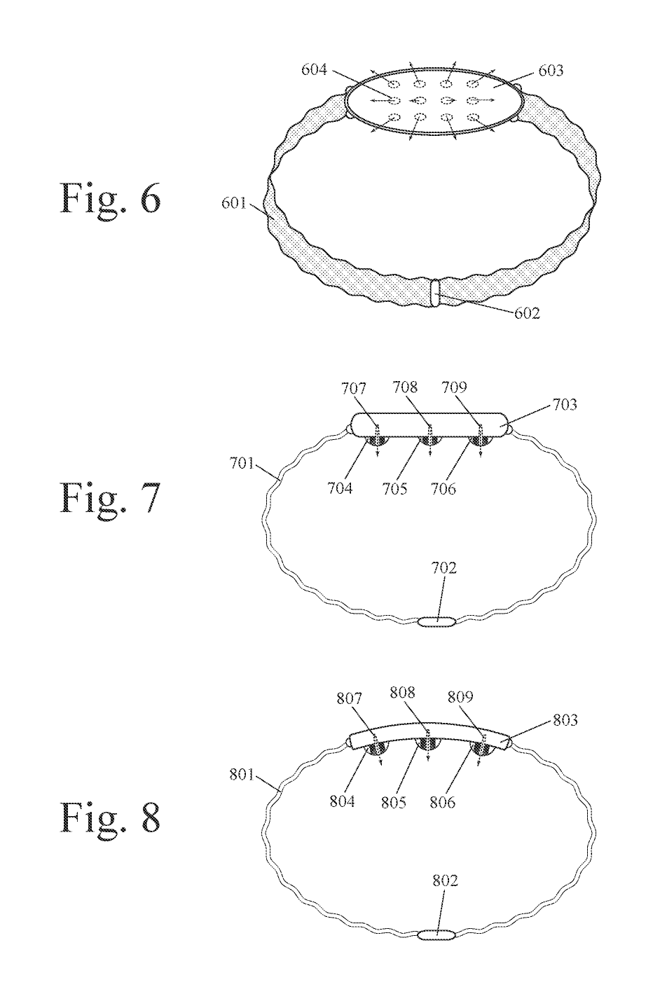

FIG. 6 shows another example of a wearable device for the arm with a plurality of close-fitting biometric sensors. This figure shows the device from a non-perpendicular lateral perspective, as it would appear encircling a lateral cross-section of a person's wrist (or other portion of the person's arm).

Described generally, the example shown in FIG. 6 is an arcuate wrist-worn device with a two-dimensional array of spectroscopic sensors. Sensors in this two-dimensional array differ in location circumferentially (they are at different locations around the circumference of the device) and laterally (they are at different locations along axes which are perpendicular to the circumference of the device). In this example, the two-dimensional sensor array is part of the circumference-center-facing surface of an enclosure which is on the anterior (upper) portion of the device. In another example, a two-dimensional sensor array can be on the circumference-center-facing surface of a band or strap.

Having a two-dimensional sensor array allows a wearable device to record biometric measurements from multiple locations on a person's wrist. This can help to find the best location on a person's wrist from which to most-accurately record biometric measurements. Having a two-dimensional sensor array can also enable a device to record biometric measurements from substantially the same location on a person's wrist even if the device is rotated around the person's wrist or slid up or down the person's arm. A different primary sensor can be automatically selected to record data when the device rotates or slides.

More specifically, the example shown in FIG. 6 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; and (c) a two-dimensional sensor array which is part of the enclosure, wherein sensors in this two-dimensional array differ in location along a portion of the circumference of the device, and wherein sensors in this two-dimensional array differ in location along axes which are perpendicular to the circumference of the device.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, there can be a display screen on the outward-facing surface of an enclosure.

In an example, sensors in a two-dimensional sensor array can be spectroscopic sensors which are each configured to measure the spectrum of light energy reflected from (and/or absorbed by) tissue of the person's arm. In an example, sensors in a two-dimensional sensor array can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 6 includes: a strap (or band) 601, a strap (or band) connector 602, an enclosure 603, and a two-dimensional spectroscopic sensor array which includes sensor 604. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 7 shows another example of a wearable device for the arm with a plurality of close-fitting biometric sensors. This figure shows the device from a side perspective, as it would appear encircling a lateral cross-section of a person's wrist (or other portion of the person's arm).

Described generally, the example shown in FIG. 7 is an arcuate wrist-worn device with a plurality of spectroscopic sensors, wherein each of these sensors is pushed toward the surface of an arm in order to stay in close contact with the surface of the arm even if the enclosure is shifted or tilted away from the surface of the arm. In this example, the spectroscopic sensors are on the circumference-center-facing portion of an enclosure. In this example, each of the spectroscopic sensors is pushed toward the surface of the arm by a spring mechanism. In another example, each of the spectroscopic sensors can be pushed toward the surface by a hydraulic mechanism, a pneumatic mechanism, or a microscale electromagnetic actuator.

More specifically, the example shown in FIG. 7 is a wearable device for the arm with a plurality of close-fitting biometric sensors comprising: (a) an attachment member, such as a strap or band, which is configured to span at least a portion of the circumference of a person's arm; (b) an enclosure which is part of (or attached to) the attachment member; and (c) a plurality of sensors which are part of the enclosure, wherein each sensor in this plurality of sensors is configured to be pushed toward the surface of the arm by a spring mechanism in order to keep the sensor in close contact with the surface of the arm.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, sensors of this device can be spectroscopic sensors which are each configured to measure the spectrum of light energy reflected from (and/or absorbed by) tissue of the person's arm. In an example, sensors of this device can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 7 includes: a strap (or band) 701; a strap (or band) connector 702; an enclosure 703; a plurality of spectroscopic sensors (707, 708, and 709); and a plurality of spring mechanisms (704, 705, and 706) which are configured to push the sensors inward toward the center of the device. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 8 shows another example of a wearable device for the arm with a plurality of close-fitting biometric sensors. This figure shows the device from a side perspective, as it would appear encircling a lateral cross-section of a person's wrist (or other portion of the person's arm). The example shown in FIG. 8 is similar to the one shown in FIG. 7, except that the enclosure housing biometric sensors in FIG. 8 has a curved circumference-center-facing surface rather than a flat circumference-center-facing surface.

With respect to specific components, the example shown in FIG. 8 includes: a strap (or band) 801; a strap (or band) connector 802; an enclosure 803; a plurality of spectroscopic sensors (807, 808, and 809); and a plurality of spring mechanisms (804, 805, and 806) which are configured to push the sensors inward toward the center of the device. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

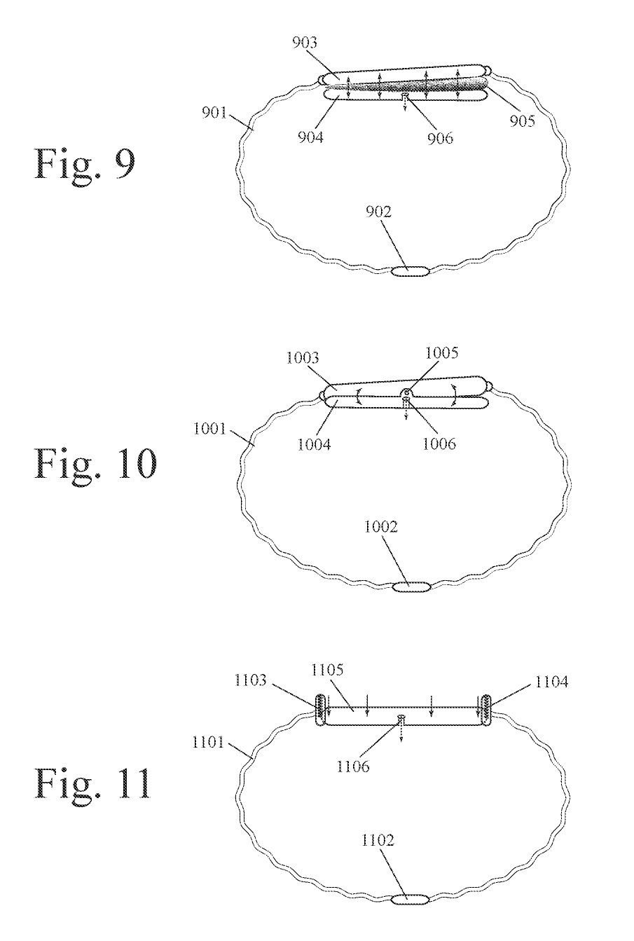

FIG. 9 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 9 is an arcuate wrist-worn device with a biometric sensor which is located on a circumference-center-facing portion of an enclosure, wherein this circumference-center-facing portion tilts on a central inflated portion of the enclosure so that the sensor remains in close contact with the surface of a person's arm even if the device tilts with respect to the arm surface. In this example, an enclosure is positioned on the anterior (upper) portion of the device circumference. In this example, the enclosure has an outward-facing portion (which can include a display screen), a central inflated portion (which can be a balloon), and an inner-facing portion (which houses the biometric sensor). In an example, a central inflated portion can be sandwiched between a rigid outward-facing portion and a rigid circumference-center-facing portion. In an example, the circumference-center-facing portion can tilt with respect to the outward-facing portion as the device tilts with respect to the surface of the person's arm.

Having a biometric sensor located on a circumference-center-facing portion of an enclosure which tilts on a central inflated portion can help to keep the biometric sensor in close proximity to the surface of the person's arm and at substantially the same angle with respect to the surface of a person's arm. This can be particularly important for a spectroscopic sensor, wherein it is desirable to maintain the same projection angle (and/or reflection angle) of a beam of light which is directed toward (and/or reflected from) the surface of a person's arm.

More specifically, the example shown in FIG. 9 is a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member, wherein this enclosure further comprises a rigid outward facing portion, an inflated central portion, and a rigid circumference-center-facing portion, wherein the rigid circumference-center-facing portion tilts relative to the rigid outward facing portion; and (c) a biometric sensor in the circumference-center-facing portion which is configured to record biometric data concerning the person's arm tissue.

In an example, there can be a display screen on the outward-facing surface of the enclosure. In an example, the central portion of an enclosure can be filled with a liquid or gel rather than inflated with a gas. In an example, there can be more than one biometric sensor on the rigid circumference-center-facing portion. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 9 includes: strap (or band) 901, strap (or band) connector 902, outward facing portion 903 of an enclosure, circumference-center-facing portion 904 of the enclosure, inflated central portion 905 of the enclosure, and a biometric sensor 906 on the circumference-center-facing portion of the enclosure. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 10 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 10 is an arcuate wrist-worn device with a biometric sensor which is located on a circumference-center-facing portion of an enclosure, wherein this circumference-center-facing portion pivots around an axis so that the sensor remains in close contact with the surface of a person's arm even if the device tilts with respect to the arm surface. In this example, an enclosure is positioned on the anterior (upper) portion of the device circumference. In this example, the enclosure has an outward-facing portion (which can include a display screen) and an inner-facing portion (which houses the biometric sensor).

In this example, a circumference-center-facing portion which houses a biometric sensor pivots around a central axis when the device tilts with respect to the surface of the person's arm. Having a biometric sensor located on a circumference-center-facing portion of an enclosure which pivots around an axis can help to keep the biometric sensor in close proximity to the surface of the person's arm and at substantially the same angle with respect to the surface of a person's arm. This can be particularly important for a spectroscopic sensor, wherein it is desirable to maintain the same projection angle (and/or reflection angle) of a beam of light which is directed toward (and/or reflected from) the surface of a person's arm.

More specifically, the example shown in FIG. 10 is a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member, wherein this enclosure further comprises an outward facing portion and a circumference-center-facing portion, wherein the rigid inward (or center) pivots around a central axis with respect to the outward facing portion; and (c) a biometric sensor in the circumference-center-facing portion which is configured to record biometric data concerning the person's arm tissue.

In this example, the central axis around which the circumference-center-facing portion pivots is perpendicular to the circumference of the device. In another example, the central axis around which the circumference-center-facing portion pivots can be parallel or tangential to the circumference of the device. In an example, there can be a display screen on the outward-facing surface of the enclosure. In an example, there can be more than one biometric sensor on the circumference-center-facing portion of the enclosure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 10 includes: strap (or band) 1001, strap (or band) connector 1002, outward facing portion 1003 of an enclosure, circumference-center-facing portion 1004 of the enclosure, axis 1005 around which circumference-center-facing portion 1004 pivots; and a biometric sensor 1006 on the circumference-center-facing portion of the enclosure. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 11 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 11 is a wrist-worn device with a biometric sensor located on an enclosure, wherein the enclosure is pushed toward the surface of a person's arm by spring mechanisms so that the sensor remains in close contact with the arm's surface even if the rest of the device shifts away from the arm's surface. In this example, the enclosure is on the anterior (upper) portion of the device circumference.

The example shown in FIG. 11 can also be expressed as a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member; (c) one or more spring mechanisms which push the enclosure inward toward the circumference center of the device; and (d) a biometric sensor in the enclosure which is configured to record biometric data concerning the person's arm tissue.

In this example, there are two spring mechanisms which push the enclosure inward toward the surface of a person's arm. In this example, these spring mechanisms are located at the places where the enclosure is connected to a strap or band. In an example, there can be a display screen on the outward-facing surface of the enclosure. In an example, there can be more than one biometric sensor on the circumference-center-facing portion of the enclosure. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 11 includes: strap (or band) 1101, strap (or band) connector 1102, first spring mechanism 1103, second spring mechanism 1104, enclosure 1105 which is pushed inward (toward the circumference center of the device) by spring mechanisms 1103 and 1104, and biometric sensor 1106. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

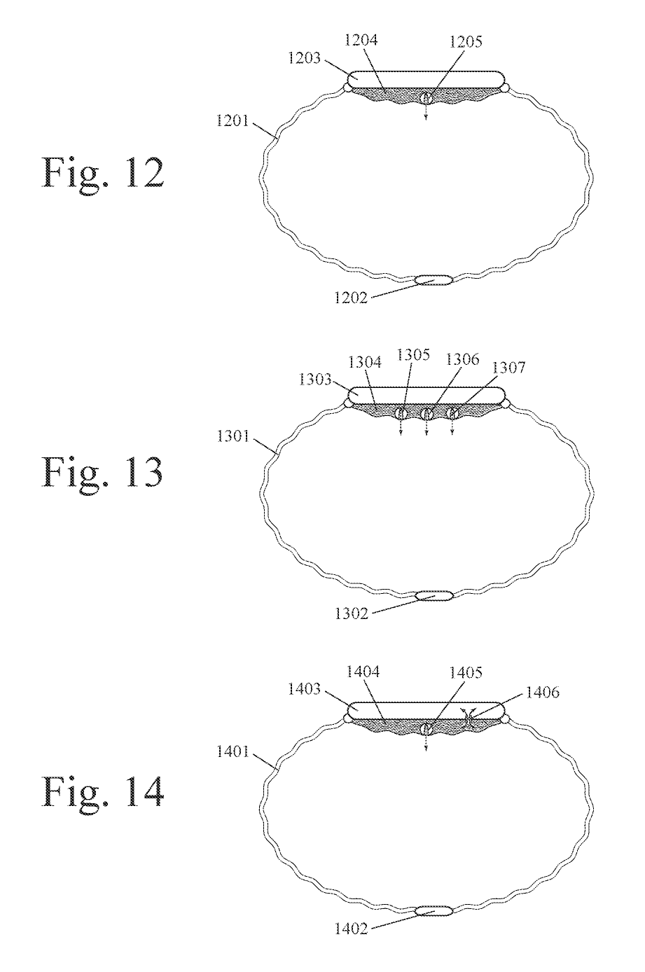

FIG. 12 shows another example of a wearable device for the arm with one or more close-fitting biometric sensors. This figure shows the device from a side perspective, as it would appear encircling a lateral cross-section of a person's wrist (or other portion of the person's arm). Described generally, this example is a wrist-worn device with an elastic member (such as a balloon) that is filled with a fluid, gel, or gas and a biometric sensor which is attached to the circumference-center-facing wall of this elastic member. Having a biometric sensor attached to the circumference-center-facing wall of an elastic member can help to keep the sensor in close contact with the surface of a person's arm, even if other components of the device are shifted or tilted away from the arm's surface. In an example, an elastic member can be part of an enclosure which is attached to an arm by a strap. In an example, such an enclosure can be positioned on the anterior (upper) portion of the device circumference.

The example shown in FIG. 12 can also be expressed as a wearable device for the arm with one or more close-fitting biometric sensors comprising: (a) an attachment member, such as a strap or band, which is configured to span at least a portion of the circumference of a person's arm; (b) an enclosure which is part of (or attached to) the attachment member; (c) an elastic member filled with a fluid, gel, or gas which is attached to (or part of) the enclosure; and (d) a biometric sensor which is configured to record biometric data concerning the person's arm tissue, wherein this sensor is attached to a circumference-center-facing wall of the elastic member.

In an example, there can be a display screen on the outward facing surface of an enclosure. In an example, there can be more than one biometric sensor on the circumference-center-facing wall of an elastic member. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 12 includes: strap (or band) 1201; strap (or band) connector 1202; enclosure 1203; elastic member 1204 which is filled with a fluid, gel, or gas; and biometric sensor 1205 which is attached to the circumference-center-facing wall of the elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 13 shows another example of a wearable device for the arm with one or more close-fitting biometric sensors. This figure shows the device from a side perspective, as it would appear encircling a lateral cross-section of a person's wrist (or other portion of the person's arm). The example shown in FIG. 13 is like the one shown in FIG. 12, except that in FIG. 13 there are multiple biometric sensors on the circumference-center-facing wall of an elastic member. In FIG. 13, there are three biometric sensors.

With respect to specific components, the example shown in FIG. 13 includes: strap (or band) 1301; strap (or band) connector 1302; enclosure 1303; elastic member 1304 which is filled with a fluid, gel, or gas; and biometric sensors 1305, 1306, and 1307 which are attached to the circumference-center-facing wall of the elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 14 shows another example of a wearable device for the arm with one or more close-fitting biometric sensors. This figure shows the device from a side perspective, as it would appear encircling a lateral cross-section of a person's wrist (or other portion of the person's arm). The example shown in FIG. 14 is like the one shown in FIG. 12, except that in FIG. 14 there is also a micropump which can pump fluid, gel, or gas into (or out of) the elastic member. This enables (automatic) adjustment of the size and/or internal pressure of the elastic member in order to better maintain proximity of the sensor to the surface of the person's arm.

With respect to specific components, the example shown in FIG. 14 includes: strap (or band) 1401; strap (or band) connector 1402; enclosure 1403; elastic member 1404 which is filled with a fluid, gel, or gas; biometric sensor 1405 which is attached to the circumference-center-facing wall of the elastic member; and micropump 1406 which pumps fluid, gel, or gas into (or out of) the elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

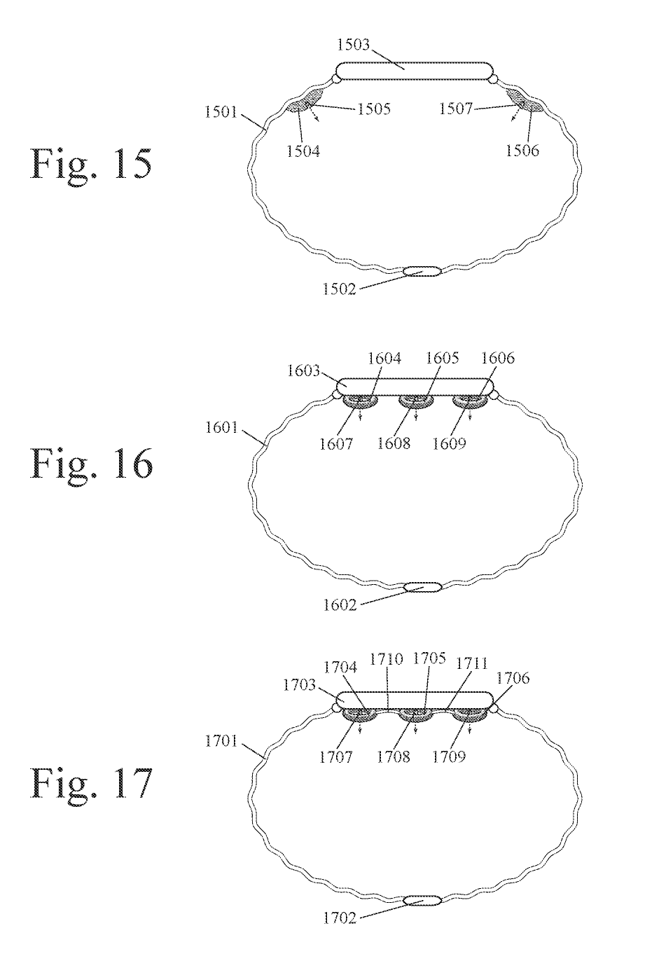

FIG. 15 shows another example of a wearable device for the arm with one or more close-fitting biometric sensors. This figure shows the device from a side perspective, as it would appear encircling a lateral cross-section of a person's wrist (or other portion of the person's arm). This wrist-worn device comprises: an attachment member which is configured to span at least a portion of the circumference of a person's arm; one or more elastic members filled with a flowable substance, wherein these elastic members are part of (or attached to) the circumference-center-facing surface of the attachment member; and one or more biometric sensors, wherein each sensor is part of (or attached to) a circumference-center-facing wall of an elastic member.

The design of this device keeps biometric sensors close to the surface of a person's arm, even if portions of the device shift away from the surface of the person's arm. The interiors of the elastic members on which these sensors are located are under modest pressure so that these elastic members expand when they are moved away from the arm surface and these elastic members are compressed when they are moved toward the arm surface.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, an attachment member can be attached to a person's arm by stretching it circumferentially and sliding it over the person's hand onto the arm. In an example, an attachment member can be attached to a person's arm by applying force to pull two ends of the member apart in order to slip the member over the arm; the two ends then retract back towards each other when device is on the arm and the force is removed.

In an example, an elastic member can be a balloon or other elastic substance-filled compartment. In an example, the flowable substance inside an elastic member can be a fluid, gel, or gas. In this example, there are two elastic members on the attachment member. In this example, the elastic members are symmetrically located with respect to a central cross-section of the device. In an example, there can be a plurality of elastic members (with attached biometric sensors) which are distributed around the circumference of an attachment member and/or the device. In this example, a device can also include an enclosure which further comprises a display screen.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 15 includes: band 1501; band connector 1502; enclosure 1503; first elastic member 1504 which is filled with a fluid, gel, or gas; first biometric sensor 1505 which is attached to the circumference-center-facing wall of the first elastic member; second elastic member 1506 which is filled with a fluid, gel, or gas; and second biometric sensor 1507 which is attached to the circumference-center-facing wall of the second elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 16 shows another example of a wearable device for the arm with one or more close-fitting biometric sensors. This figure shows the device from a side perspective, as it would appear encircling a lateral cross-section of a person's wrist (or other portion of the person's arm). This wrist-worn device comprises: (a) an attachment member which is configured to span at least a portion of the circumference of a person's arm; (b) an enclosure which is part of (or attached to) the attachment member; (c) one or more torus-shaped elastic members filled with a flowable substance, wherein these elastic members are part of (or attached to) the enclosure; and (d) one or more biometric sensors, wherein each sensor is located in the central hole of a torus-shaped elastic member.

In an example, an attachment member can be a strap, band, bracelet, ring, armlet, cuff, or sleeve. In an example, an enclosure can further comprise a display screen on its outer surface. In an example, a torus-shaped elastic member can be a balloon which is filled with a fluid, gel, or gas. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 16 includes: band 1601; band connector 1602; enclosure 1603; torus-shaped elastic members 1604, 1605, and 1606; and biometric sensors 1607, 1608, and 1609 which are each located in the central opening (or hole) of a torus-shaped elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 17 shows another example of a wearable device for the arm with one or more close-fitting biometric sensors. This figure shows the device from a side perspective, as it would appear encircling a lateral cross-section of a person's wrist (or other portion of the person's arm). The example in FIG. 17 like the one shown in FIG. 16, except that the example in FIG. 17 also includes channels through which a fluid, gel, or gas can flow between the torus-shaped elastic members.

With respect to specific components, the example shown in FIG. 17 includes: band 1701; band connector 1702; enclosure 1703; torus-shaped elastic members 1704, 1705, and 1706; biometric sensors 1707, 1708, and 1709 which are each located in the central opening (or hole) of a torus-shaped elastic member; and channels 1710 and 1711 through which fluid, gel, or gas can flow between the torus-shaped elastic members. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

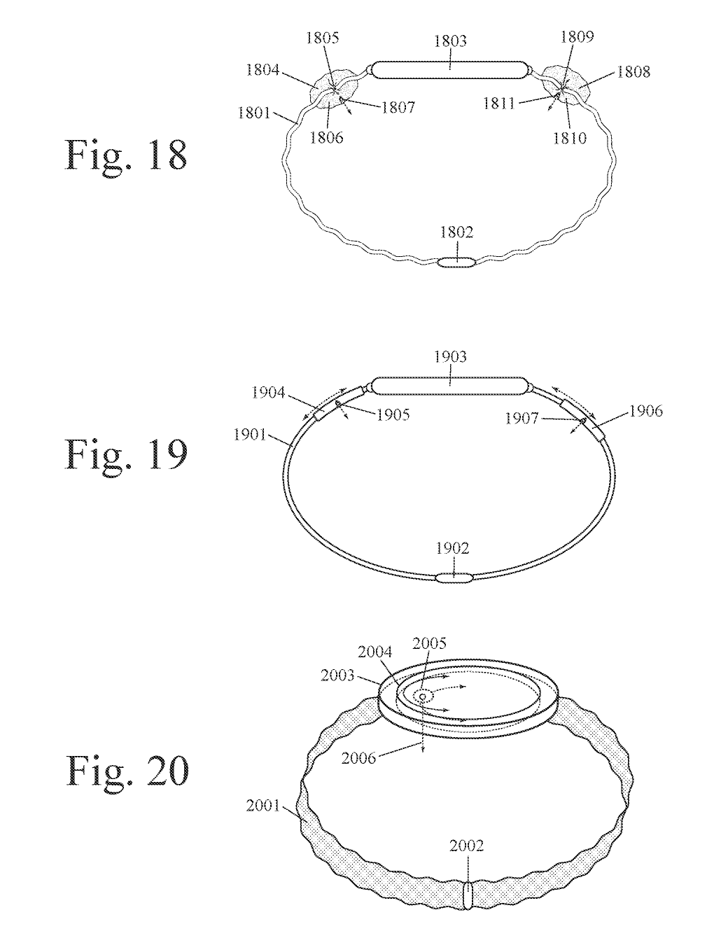

FIG. 18 shows another example of a wearable device for the arm with one or more close-fitting biometric sensors. This figure shows the device from a side perspective, as it would appear encircling a lateral cross-section of a person's wrist (or other portion of the person's arm). The example in FIG. 18 like the one shown in FIG. 15, except that the example in FIG. 18 also includes elastic members on the outward-facing surface of the attachment member and channels through which fluid, gel, or gas can flow from circumference-center-facing elastic members to outward-facing elastic members, or vice versa.

This wrist-worn device comprises: (a) an attachment member which is configured to span at least a portion of the circumference of a person's arm; (b) at least one circumference-center-facing elastic member, wherein this member is filled with a flowable substance, and wherein this elastic member is part of (or attached to) the circumference-center-facing surface of the attachment member; (c) at least one outward-facing elastic member, wherein this member is filled with the flowable substance, and wherein this elastic member is part of (or attached to) the outward-facing surface of the attachment member; (d) a channel through which the flowable substance can flow between the circumference-center-facing elastic member and the outward-facing elastic member; and (e) a biometric sensor which is part of (or attached to) the circumference-center-facing wall of the circumference-center-facing elastic member.

In an example, an attachment member can be a strap, band, bracelet, ring, armlet, cuff, or sleeve. In an example, one or both of the elastic members can be a balloon or other elastic substance-filled compartment. In an example, the flowable substance inside an elastic member can be a fluid, gel, or gas. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 18 includes: band 1801; band connector 1802; enclosure 1803; outward-facing elastic members 1804 and 1808, which are filled with a fluid, gel, or gas; circumference-center-facing elastic members 1806 and 1810, which are filled with the fluid, gel, or gas; channels 1805 and 1809 through which the fluid, gel, or gas can flow from an outward-facing elastic member to a circumference-center-facing elastic member, or vice versa; and biometric sensors 1807 and 1811 which are each attached to a circumference-center-facing wall of a circumference-center-facing elastic member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 19 shows another example of a wearable device for the arm with one or more close-fitting biometric sensors. This figure shows the device from a side perspective, as it would appear encircling a lateral cross-section of a person's wrist (or other portion of the person's arm). A general description of the example in FIG. 19 can be expressed as a wrist-worn device with biometric sensors on circumferentially-sliding members, wherein these circumferentially-sliding members are slid along the circumference of the device in order to adjust the positions from which the biometric sensors measure data concerning arm tissue. Such moveable sensors enable a user to find the best positions around the circumference of the device from which to collect biometric data for a selected application.

This wrist-worn device comprises: (a) an attachment member which is configured to span at least a portion of the circumference of a person's arm; (b) at least one circumferentially-sliding member, wherein this member is slid along the circumference of the attachment member; and (c) at least one a biometric sensor which is part of (or attached to) the circumferentially-sliding member and collects data concerning arm tissue.

In an example, a sliding member can laterally-encircle an attachment member in order to keep the sliding member on the attachment member. In an example, the ends of a sliding member can curve around the sides of an attachment member in order to keep the sliding member on the attachment member. In an example, there can be a circumferential track on an attachment member into which a sliding member fits in order to keep the sliding member on the attachment member. In an example, a spring or other compressive mechanism on a sliding member can engage the attachment member in order to keep the sliding member on the attachment member. In an example, pressing on the top or sides of a sliding member frees it to slide along the attachment member and releasing this pressure causes the sliding member to stop sliding (and remain at a selected location on the attachment member). In an example, data from a biometric sensor on the sliding member can be analyzed in real time in order to identify the optimal location along the circumference of the attachment member from which to collect data.

In an example, an attachment member can be a strap, band, bracelet, ring, armlet, cuff, or sleeve. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 19 includes: band 1901; band connector 1902; enclosure 1903; circumferentially-sliding members 1904 and 1906, wherein these members slide along the circumference of the band; and biometric sensors 1905 and 1907 which are each part of (or attached to) a circumferentially-sliding member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 20 shows another example of a wearable device for the arm with one or more close-fitting biometric sensors. This figure shows the device from a non-perpendicular lateral perspective, as it would appear encircling a lateral cross-section of a person's wrist (or other portion of the person's arm).

A general description of the example in FIG. 20 can be expressed as a wrist-worn device with a biometric sensor on a rotating member, wherein rotation of the rotating member moves the biometric sensor in a circular manner. In an example, the circular path in which a sensor moves is configured to be in a plane which is substantially tangential to the surface of a person's arm. In an example, a user can manually rotate the rotating member in order to find the optimal location from which to have the sensor collect biometric data. In an example, a device can automatically rotate the rotating member in order to find the optimal location from which to have the sensor collect biometric data. In an example, a device can automatically rotate the rotating member in order to maintain the optimal sensor location if the device is unintentionally shifted with respect to the arm's surface. In an example, a device can automatically rotate the rotating member in order to collect data from multiple locations for more comprehensive and/or accurate analysis.

In this example, a wearable device for the arm with one or more close-fitting biometric sensors comprises: (a) an attachment member which is configured to span at least a portion of the circumference of a person's arm; (b) an enclosure which is part of (or attached to) the attachment member; (c) a rotating member which is part of (or attached to) the enclosure; and (d) a biometric sensor which is part of (or attached to) the rotating member, wherein this biometric sensor is configured to collect data concerning a person's arm tissue, and wherein this biometric sensor moves in a circular path when the rotating member is rotated.

In an example, an attachment member can be a strap, band, bracelet, ring, armlet, cuff, or sleeve. In an example, a rotating member can be a circular member which fits into a hole or recess in an enclosure. In an example, a rotating member can be manually moved by a user in order to find the best location from which to have a sensor collect biometric data. In an example, a rotating member can be automatically moved by an actuator in the device in order to find the best location from which to have a sensor collect biometric data. In an example, a rotating member can be automatically moved by an actuator in the device in order to maintain the best sensor location when an enclosure is unintentionally shifted with respect to the arm's surface. In an example, a rotating member can be automatically moved by an actuator in order to collect data from multiple locations for more comprehensive and/or accurate analysis.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 20 includes: band 2001; band connector 2002; enclosure 2003; rotating member 2004 which is part of (or attached to) the enclosure; and biometric sensor 2005 which is part of (or attached to) the rotating member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

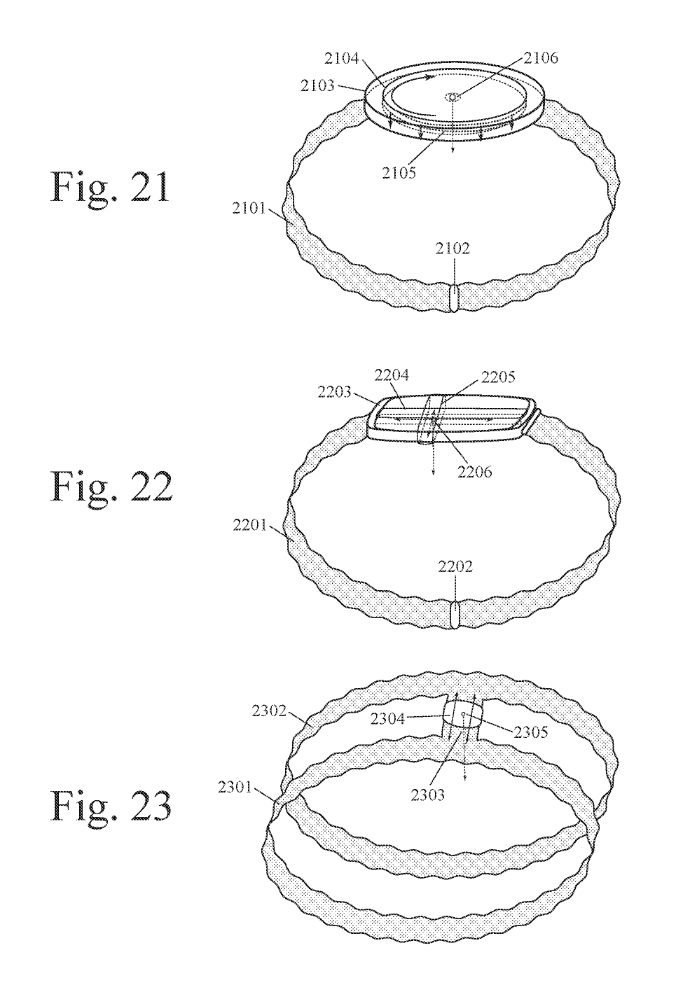

FIG. 21 shows another example of a wearable device for the arm with one or more close-fitting biometric sensors. This figure shows the device from a non-perpendicular lateral perspective, as it would appear encircling a lateral cross-section of a person's wrist (or other portion of the person's arm).

A general description of the example shown in FIG. 21 can be expressed as a wrist-worn device with a biometric sensor on a threaded rotating member, wherein rotation of the threaded rotating member adjusts the distance between the biometric sensor and the surface of a person's arm. In an example, a user can manually rotate the rotating member in order to find the optimal distance between the sensor and the arm's surface from which to have the sensor collect biometric data. In an example, a device can automatically (e.g. with an actuator) rotate the rotating member in order to find the optimal distance between the sensor and the arm's surface from which to have the sensor collect biometric data. In an example, a device can automatically (e.g. with an actuator) rotate the rotating member to maintain the optimal distance between a sensor and the arm's surface if the enclosure is unintentionally shifted with respect to the arm's surface.

In this example, a wearable device for the arm with one or more close-fitting biometric sensors comprises: (a) an attachment member which is configured to span at least a portion of the circumference of a person's arm; (b) an enclosure which is attached to (or part of) the attachment member; (c) a threaded rotating member which is attached to (or part of) the enclosure, wherein rotation of the threaded rotating member changes the distance between the threaded rotating member and the circumferential center of the device; and (d) a biometric sensor which is attached to (or part of) the threaded rotating member, wherein this biometric sensor is configured to collects data concerning a person's arm tissue, and wherein rotation of the threaded rotating member changes the distance between the biometric sensor and the circumferential center of the device. In an example, rotation of the threaded rotating member is also configured to change the distance between the biometric sensor and the surface of the person's arm.

In an example, an attachment member can be a strap, band, bracelet, ring, armlet, cuff, or sleeve. In an example, a threaded rotating member can have a spiral thread around its circumference which fits into a complementary spiral thread in a hole or recess in the enclosure. In an example, a threaded rotating member can be manually moved by a user in order to find the best distance between a sensor and the arm's surface from which to collect biometric data. In an example, a threaded rotating member can be automatically moved by an actuator in the device in order to find the best distance between a sensor and the arm's surface from which to collect biometric data. In an example, a threaded rotating member can be automatically moved by an actuator in the device in order to maintain the best distance between a sensor and the arm's surface when the location of an enclosure with respect to the arm's surface is unintentionally shifted.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 21 includes: band 2101; band connector 2102; enclosure 2103; threaded rotating member 2104 with spiral thread 2105 which is part of (or attached to) the enclosure; and biometric sensor 2106 which is part of (or attached to) the rotating member. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 22 shows another example of a wearable device for the arm with one or more close-fitting biometric sensors. This figure shows the device from a non-perpendicular lateral perspective, as it would appear encircling a lateral cross-section of a person's wrist (or other portion of the person's arm). The example shown in FIG. 22 can be described as a wrist-worn device with a biometric sensor, wherein the location of this sensor can be moved along an X axis and/or along a Y axis, wherein the X axis is substantially tangential to the circumference of the device, and wherein the Y axis is perpendicular to the X axis.

In an example, a user can manually move a sensor along these X and/or Y axes in order to find the optimal location from which to collect biometric data concerning arm tissue. In an example, the device can automatically move a sensor (e.g. with an actuator) along these X and/or Y axes in order to find the optimal location from which to collect biometric data concerning arm tissue. In an example, the device can automatically move a sensor (e.g. with an actuator) along these X and/or Y axes in order to keep the sensor at the optimal location even if the device is unintentionally shifted with respect to the arm's surface. In an example, the device can automatically move a sensor (e.g. with an actuator) along these X and/or Y axes in order to collect data from various locations for more comprehensive or accurate analysis.

In this example, a wearable device for the arm with one or more close-fitting biometric sensors comprises: (a) an attachment member which is configured to span at least a portion of the circumference of a person's arm; (b) an enclosure which is attached to (or part of) the attachment member; (c) a biometric sensor which is configured to collect data concerning arm tissue; (d) a first moving member whose movement moves the biometric sensor along an X axis, wherein this X axis is substantially tangential to the circumference of the device; and (e) a second moving member whose movement moves the biometric sensor along an Y axis, wherein this Y axis is perpendicular to the X axis.

In an example, an attachment member can be a strap, band, bracelet, ring, armlet, cuff, or sleeve. In an example, a biometric sensor can be attached to a circumference-center-facing portion of an enclosure. In an example, first and second moving members can be sliding members. In an example, a first moving member can be a strip on an enclosure which slides along the X axis. In an example, a second moving member can be a strip on an enclosure which slides along the Y axis. In another example, first and second moving members can be rotating members.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 22 includes: band 2201; band connector 2202; enclosure 2203; first sliding member 2204 which slides along an X axis, wherein the X axis is substantially tangential to the circumference of the device; second sliding member 2205 which slides along an Y axis, wherein the Y axis is substantially perpendicular to the X axis; and biometric sensor 2206 which is configured to collect data concerning arm tissue, wherein the X and Y coordinates of biometric sensor 2206 are changed by moving the first and second sliding members, respectively. In a variation on this example, both an enclosure and Y axis can be arcuate. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 23 shows another example of a wearable device for the arm with one or more close-fitting biometric sensors. This figure shows the device from a non-perpendicular lateral perspective, as it would appear encircling a lateral cross-section of a person's wrist (or other portion of the person's arm). The example shown in FIG. 23 can be described as a wrist-worn device with: two parallel bands which are connected to each other on the anterior (upper) surface of the wrist; and a biometric sensor which slides (back and forth) along the strip which connects the two bands.

In an example, a user can manually slide the biometric sensor (back and forth) along the strip connecting the two bands in order to find the optimal location from which to collect biometric data concerning arm tissue. In an example, the device can automatically slide the biometric sensor (back and forth) along the strip connecting the two bands in order to find the optimal location from which to collect biometric data concerning arm tissue. In an example, the device can automatically slide the biometric sensor (back and forth) along the strip connecting the two bands in order to collect data from different locations for more comprehensive or accurate analysis.

In this example, a wearable device for the arm with one or more close-fitting biometric sensors comprises: (a) two substantially-parallel bands which are each configured to span at least a portion of the circumference of a person's arm; (b) a connecting strip which is configured to connect the two bands to each other on the anterior (upper) surface of the arm; (c) a moving enclosure which slides (back and forth) along the connecting strip; and (d) a biometric sensor which is configured to collect data concerning arm tissue, wherein this biometric sensor is part of (or attached to) the moving enclosure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 23 includes: first band 2301; second band 2302; connecting strip 2303 which connects the first and second bands; sliding enclosure 2304 which slides (back and forth) along the connecting strip; and biometric sensor 2305 which is configured to collect data concerning arm tissue, wherein this biometric sensor is part of (or attached to) the sliding enclosure. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

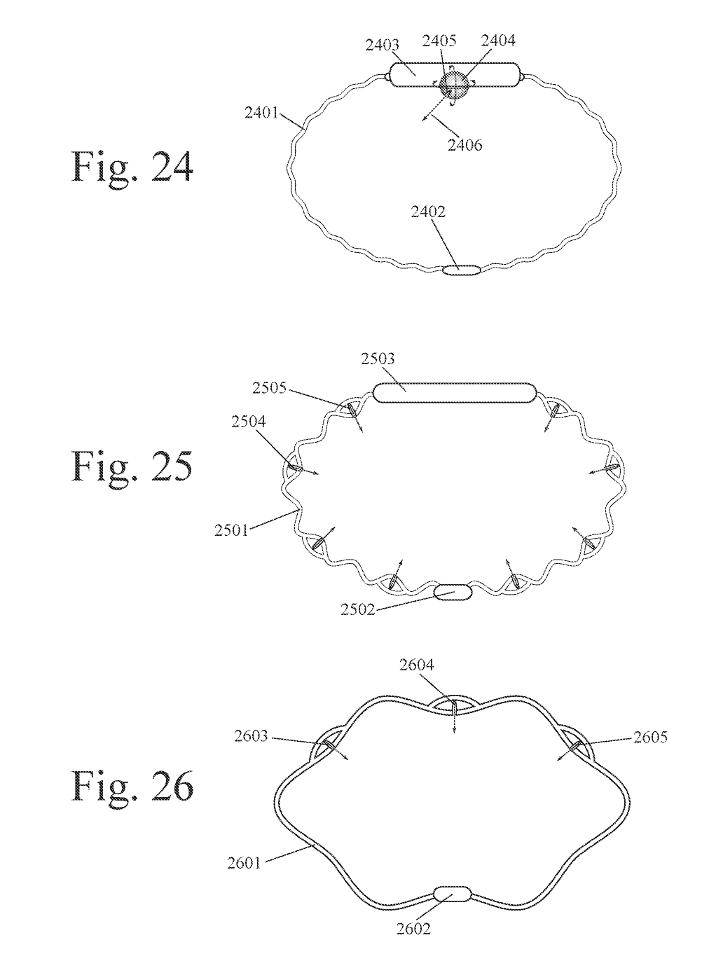

FIG. 24 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 24 is an arcuate wrist-worn device with a light-projecting spectroscopic sensor on a rotating ball. Rotating the ball changes the angle at which the spectroscopic sensor projects light onto the surface of a person's arm. The ball can be rotated in different directions so that the range of possible projection beams comprises a conic or frustal shape in three-dimensional space. Having a light-projecting spectroscopic sensor on a rotating ball can enable a device to record biometric measurements with substantially the same angle of incidence, even if an enclosure is tilted with respect to the surface of the person's arm.

The example shown in FIG. 24 is a wearable device for the arm with a close-fitting biometric sensor comprising: (a) an attachment member, such as a strap or band, which is configured to span at least a portion of the circumference of a person's arm; (b) an enclosure which is part of (or attached to) the attachment member; (c) a rotating ball which is part of (or attached to) the enclosure; and (d) a light-projecting spectroscopic sensor which is part of (or attached to) the rotating ball.

In an example, an attachment member can be a strap, band, bracelet, ring, armlet, cuff, or sleeve. In an example, the circumference-center-facing surface of an enclosure can be substantially flat. In an example, the circumference-center-facing surface of an enclosure can be curved. In an example, there can be a display screen on the outward-facing surface of an enclosure. In an example, the rotating ball can fit into the enclosure like a ball-and-socket joint. In an example, the device can further comprise one or more actuators which move the rotating ball.

With respect to specific components, the example shown in FIG. 24 includes: strap 2401, strap connector 2402, enclosure 2403, rotating ball 2404, and spectroscopic sensor 2405 which emits beam of light 2406. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 25 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 25 is a wearable device for the arm with a flexible circumferentially-undulating band with biometric sensors on the proximal portions of undulating waves. A band with such a flexible circumferentially-undulating structure can help to keep a plurality of biometric sensors in close proximity to the surface of a person's arm. In an example, an attachment member can be a strap, band, bracelet, ring, or armlet. In an example, a circumferentially-undulating attachment member can have a repeating wave pattern. In an example, a circumferentially-undulating attachment member can have a sinusoidal wave pattern.

The example shown in FIG. 25 is a wearable device for the arm with a close-fitting biometric sensor comprising: (a) a circumferentially-undulating attachment member which is configured to span at least a portion of the circumference of a person's arm; and (b) a plurality of biometric sensors which collect data concerning arm tissue, wherein each biometric sensor is located at the proximal portion of an undulation, and wherein the proximal portion of an undulation is the portion of an undulating wave which is closest to the circumferential center of the device.

With respect to specific components, the example shown in FIG. 25 includes: circumferentially-undulating band 2501, band connector 2502, enclosure 2503, first biometric sensor 2504 at the proximal portion of a first wave in the circumferentially-undulating band, and second biometric sensor 2505 at the proximal portion of a second wave in the circumferentially-undulating band. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 26 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 26 is a wearable device for the arm with a flexible circumferentially-undulating band with six waves and biometric sensors on the proximal portions of some or all of these waves.

A band with a circumferentially-undulating structure can help to keep a plurality of biometric sensors in close proximity to the surface of a person's arm. Further, a band with six waves can engage the sides of a person's wrist with two symmetrically-opposite waves to resist rotational shifting better than a circular or oval band. This can help to reduce measurement errors caused by movement of biometric sensors. In an example, a circumferentially-undulating attachment member can be a strap, band, bracelet, ring, or armlet. In an example, a circumferentially-undulating attachment member can have a repeating wave pattern. In an example, a circumferentially-undulating attachment member can have a sinusoidal wave pattern.

The example shown in FIG. 26 is a wearable device for the arm with one or more close-fitting biometric sensors comprising: (a) a circumferentially-undulating attachment member with six waves which is configured to span the circumference of a person's arm; and (b) a plurality of biometric sensors which collect data concerning arm tissue, wherein each biometric sensor is located at the proximal portion of an undulation, and wherein the proximal portion of an undulation is the portion of an undulating wave which is closest to the circumferential center of the device.

With respect to specific components, the example shown in FIG. 26 includes: circumferentially-undulating band 2601 with six waves, band connector 2602, a first biometric sensor 2603 at the proximal portion of a first wave in the circumferentially-undulating band, a second biometric sensor 2605 at the proximal portion of a second wave in the circumferentially-undulating band, and a third biometric sensor 2606 at the proximal portion of a third wave in the circumferentially-undulating band. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

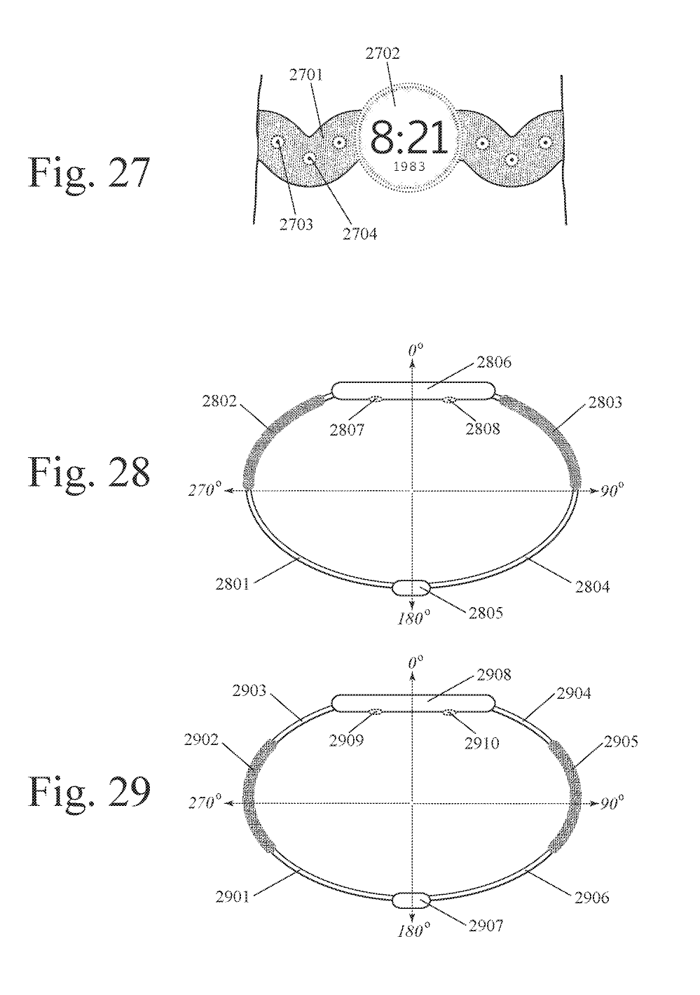

FIG. 27 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. Described generally, the example shown in FIG. 27 is a wearable device for the arm with a laterally-undulating band and biometric sensors. Lateral undulations are waves which are substantially perpendicular to the plane containing the band circumference. In an example, a band can have sinusoidal lateral undulations.

The example shown in FIG. 27 is a wearable device for the arm with one or more close-fitting biometric sensors comprising: (a) a laterally-undulating attachment member which is configured to span at least a portion of the circumference of a person's arm, wherein lateral undulations are waves which are substantially perpendicular to the plane containing the circumference of the attachment member; and (b) one or more biometric sensors which collect data concerning arm tissue, wherein these biometric sensors are part of (or attached to) the laterally-undulating attachment member.

With respect to specific components, the example shown in FIG. 27 includes: laterally-undulating strap 2701; display screen 2702; and biometric sensors including 2703 and 2704. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 28 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 28 is a wearable device for an arm with one or more biometric sensors in an enclosure and an attachment member (such as a strap, band, bracelet, or cuff) which attaches the enclosure to the arm, wherein this attachment member has relatively-elastic portions connected to the enclosure and relatively-inelastic portions elsewhere. This structure can help to keep the enclosure and sensors fitting closely against the arm. This, in turn, can enable more-consistent collection of data concerning arm tissue.

In an example, the device in FIG. 28 can be specified as a wearable device for the arm with one or more close-fitting biometric sensors comprising: (a) an attachment member which is configured to span at least 60% of the circumference of a person's arm, wherein this attachment member further comprises—one or more elastic portions which are configured to span the anterior (upper) surface of a person's arm and one or more inelastic portions which are configured to span the posterior (lower) surface of the person's arm; (b) an enclosure which is connected to the elastic portions of the attachment member; and (c) one or more biometric sensors which collect data concerning arm tissue which are part of (or attached to) the enclosure.

In an alternative example, a wearable device for the arm with one or more close-fitting biometric sensors can comprise: (a) an attachment member which is configured to span at least 60% of the circumference of a person's arm, wherein this attachment member further comprises—one or more elastic portions which are configured to span the posterior (lower) surface of a person's arm and one or more inelastic portions which are configured to span the anterior (upper) surface of the person's arm; (b) an enclosure which is connected to the elastic portions of the attachment member; and (c) one or more biometric sensors which collect data concerning arm tissue which are part of (or attached to) the enclosure.

In an example, an elastic portion of an attachment member can be an elastic strap or band. In an example, an elastic portion of an attachment member can be made from elastic fabric. In an example, an elastic portion of an attachment member can have a first elasticity level, an inelastic portion of an attachment member can have a second elasticity level, and the first elasticity level can be greater than the second elasticity level. In an example, a first elastic portion of an attachment member can be directly connected to a first side of an enclosure and a second elastic portion of an attachment member can be directly connected to a second (opposite) side of the enclosure. In an example, a first elastic portion of an attachment member can be indirectly connected to a first side of an enclosure and a second elastic portion of an attachment member can be indirectly connected to a second (opposite) side of the enclosure.

In an example, the device in FIG. 28 can be specified as a wearable device for the arm with one or more close-fitting biometric sensors comprising: (a) an attachment member which is configured to span at least 60% of the circumference of a person's arm wherein this attachment member further comprises: two elastic portions which are configured to span a first portion of the circumference of a person's arm; and two inelastic portions which are configured to span a second portion of the circumference of the person's arm; (b) an enclosure which is connected between the two elastic portions; (c) a clip, buckle, clasp, pin, or hook-and-eye mechanism between the two inelastic portions; and d) one or more biometric sensors which collect data concerning arm tissue which are part of (or attached to) the enclosure.

In an example, the device in FIG. 28 can be specified as a wearable device for the arm with one or more close-fitting biometric sensors comprising: (a) an attachment member which is configured to span at least 60% of the circumference of a person's arm wherein this attachment member further comprises: two elastic portions of the attachment member which are configured to span a portion of the circumference of a person's arm; and one or more inelastic portions which comprise the remainder of the attachment member; (b) an enclosure which is connected between the two elastic portions; (c) one or more biometric sensors which collect data concerning arm tissue which are part of (or attached to) the enclosure.

In an example, a single elastic portion can be configured to span at least 10% of the circumference of a person's arm. In an example, a single elastic portion can be configured to span at least 10% of the circumference of an attachment member. In an example, a single inelastic portion can be configured to span at least 10% of the circumference of a person's arm. In an example, a single inelastic portion can be configured to span at least 10% of the circumference of an attachment member. In an example, two elastic portions can be configured to collectively span at least 20% of the circumference of a person's arm. In an example, two elastic portions can be configured to collectively span at least 20% of the circumference of an attachment member. In an example, two inelastic portions can be configured to collectively span at least 20% of the circumference of a person's arm. In an example, two inelastic portions can be configured to collectively span at least 20% of the circumference of an attachment member.

In an example, a first definition of polar (or compass) coordinates can be defined for a device relative to how the device is configured to be worn on a person's arm. A 0-degree position can be defined as the position on a device circumference which is configured to intersect the longitudinal mid-line of the anterior (upper) surface of the arm. A 180-degree position is diametrically opposite (through the circumferential center) the 0-degree position. A 90-degree position is (clockwise) midway between the 0-degree and 180-degree positions. A 270-degree position is diametrically opposite the 90-degree position.

Using this first definition of polar coordinates, the device in FIG. 28 can be specified as a wearable device for the arm with one or more close-fitting biometric sensors comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90-degree positions, an inelastic third portion with a third level of elasticity which spans at least 35 degrees (clockwise) between the 90-degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is configured to be worn (clockwise) between the 270-degree and 90-degree positions; and (c) one or more biometric sensors which collect data concerning arm tissue which are part of (or attached to) the enclosure.

Using this first definition of polar coordinates, the device in FIG. 28 can also be specified as a wearable device for the arm with one or more close-fitting biometric sensors comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90-degree positions, an inelastic third portion with a third level of elasticity which spans at least 35 degrees (clockwise) between the 90-degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is connected between the elastic first and second portions; and (c) one or more biometric sensors which collect data concerning arm tissue which are part of (or attached to) the enclosure.

Alternatively, a second definition of polar (or compass) coordinates can be defined for the circumference of such a device relative to the position of an enclosure. The 0-degree position can be defined as the position on the device circumference which intersects the (lateral) mid-line of the enclosure. The 180-degree position is diametrically opposite (through the circumferential center) the 0-degree position. The 90-degree position is clockwise midway between the 0-degree and 180-degree positions. The 270-degree position is diametrically opposite the 90-degree position.

Using this second definition of polar coordinates, the device in FIG. 28 can be specified as a wearable device for the arm with one or more close-fitting biometric sensors comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90-degree positions, an inelastic third portion with a third level of elasticity which spans at least 35 degrees (clockwise) between the 90-degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is connected between the elastic first and second portions of the attachment member; and (c) one or more biometric sensors which collect data concerning arm tissue which are part of (or attached to) the enclosure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 28 includes: inelastic portion 2801 of an attachment member; elastic portion 2802 of an attachment member; elastic portion 2803 of an attachment member; inelastic portion 2804 of an attachment member; attachment member connector 2805; enclosure 2806; and biometric sensors 2807 and 2808. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 29 shows another example of a wearable device for the arm with one or more close-fitting biometric sensors. This figure shows the device from a side perspective, as it would appear encircling a lateral cross-section of a person's wrist (or other portion of the person's arm). Described generally, the example shown in FIG. 29 is a wearable device for the arm with one or more biometric sensors in an enclosure and an attachment member (such as a strap, band, bracelet, or cuff) which attaches the enclosure to the arm, wherein the attachment member is configured to have elastic portions spanning the lateral surfaces of the arm and inelastic portions spanning the anterior (upper) and posterior (lower) surfaces of the arm. This structure can help to keep the enclosure and sensors from rotating around the arm. This, in turn, can enable more-consistent collection of data concerning arm tissue.

In an example, the device in FIG. 29 can be specified as a wearable device for the arm with one or more close-fitting biometric sensors comprising: (a) an attachment member which is configured to span at least 60% of the circumference of a person's arm wherein this attachment member further comprises—one or more anterior inelastic portions which are configured to span the anterior (upper) surface of a person's arm, one or more posterior inelastic portions which are configured to span the posterior (lower) surface of a person's arm, and one or more elastic portions which connect the anterior and posterior inelastic portions; (b) an enclosure which is configured to be worn on the anterior (upper) portion of the arm; and (c) one or more biometric sensors which collect data concerning arm tissue which are part of (or attached to) the enclosure.

In another example, a wearable device for the arm with one or more close-fitting biometric sensors can comprise: (a) an attachment member which is configured to span at least 60% of the circumference of a person's arm wherein this attachment member further comprises—one or more anterior inelastic portions which are configured to span the anterior (upper) surface of a person's arm, one or more posterior inelastic portions which are configured to span the posterior (lower) surface of a person's arm, and one or more elastic portions which connect the anterior and posterior inelastic portions; (b) an enclosure which is configured to be worn on the posterior (lower) portion of the arm; and (c) one or more biometric sensors which collect data concerning arm tissue which are part of (or attached to) the enclosure.

In an example, a first inelastic portion of an attachment member can be connected to a first side of an enclosure and a second inelastic portion of an attachment member can be connected to a second side of the enclosure. In an example, an elastic portion can have a first level of elasticity, an inelastic portion can have a second level of elasticity, and the first level is greater than the second level. In an example, a single elastic portion can be configured to span at least 10% of the circumference of a person's arm. In an example, a single elastic portion can be configured to span at least 10% of the circumference of an attachment member. In an example, a single inelastic portion can be configured to span at least 10% of the circumference of a person's arm. In an example, a single inelastic portion can be configured to span at least 10% of the circumference of an attachment member.

In an example, polar (or compass) coordinates can be defined for a device relative to how the device is configured to be worn on a person's arm. A 0-degree position can be defined as the position on a device circumference which is configured to intersect the longitudinal mid-line of the anterior (upper) surface of the arm. A 180-degree position is diametrically opposite (through the circumferential center) the 0-degree position. A 90-degree position is clockwise midway between the 0-degree and 180-degree positions. A 270-degree position is diametrically opposite the 90-degree position.

In an example, the device in FIG. 29 can be specified as a wearable device for the arm with one or more close-fitting biometric sensors comprising: (a) an attachment member which is configured to span at least 60% of the circumference of a person's arm wherein this attachment member further comprises—a inelastic first portion with a first level of elasticity which spans at least 35 degrees (clockwise) between the 270-degree and 90-degree positions; an inelastic second portion with a second level of elasticity which spans at least 35 degrees (clockwise) between the 90-degree and 270-degree positions, an elastic third portion with a third level of elasticity which spans at least 35 degrees (clockwise) between the 180-degree and 0-degree positions, an elastic fourth portion with a fourth level of elasticity which spans at least 35 degrees (clockwise) between the 0-degree and 180-degree positions, and wherein each of the first and second elasticity levels is lower than each of the third and fourth elasticity levels; (b) an enclosure that is connected between the inelastic first portion and the inelastic second portion; and (c) one or more biometric sensors which collect data concerning arm tissue which are part of (or attached to) the enclosure.

In an alternative example, polar (or compass) coordinates can be defined for the circumference of such a device relative to the position of an enclosure on the device. The 0-degree position can be defined as the position on the device circumference which intersects the (lateral) mid-line of the enclosure. The 180-degree position is diametrically opposite (through the circumferential center) the 0-degree position. The 90-degree position is clockwise midway between the 0-degree and 180-degree positions. The 270-degree position is diametrically opposite the 90-degree position.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 29 includes: inelastic portion 2901 of an attachment member; elastic portion 2902 of an attachment member; inelastic portion 2903 of an attachment member; inelastic portion 2904 of an attachment member; elastic portion 2905 of an attachment member; inelastic portion 2906 of an attachment member; attachment member connector 2907; enclosure 2908; and biometric sensors 2909 and 2910. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

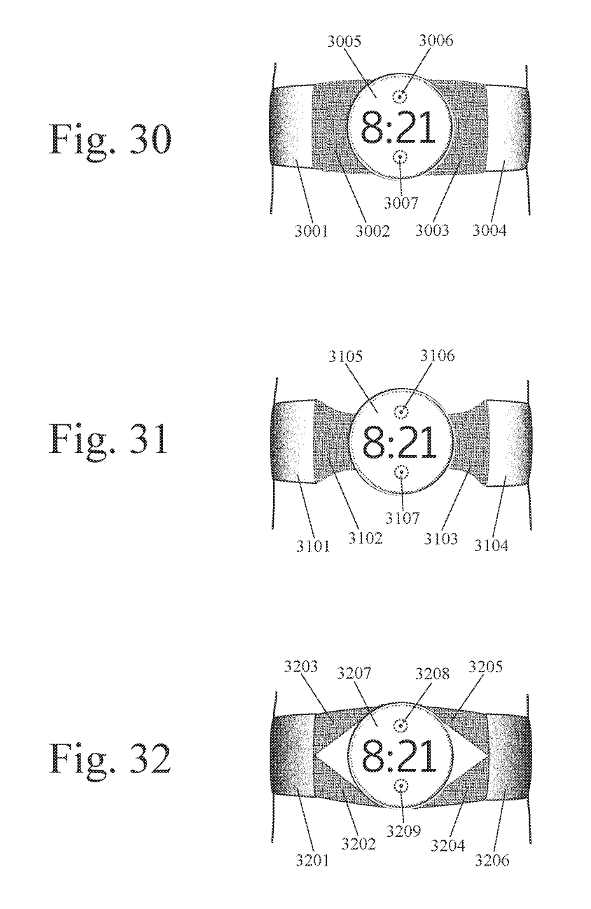

FIG. 30 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. This can be seen as a top-down view of a further-specified variation of the example that was shown from a side perspective in FIG. 28.

The example shown in FIG. 30 can be described generally as a wearable device for the arm with one or more biometric sensors in an enclosure and an attachment member (such as a band, strap, bracelet, or cuff) which holds the enclosure on a person's arm, wherein there are rectangular, rounded rectangular, or plano-concave elastic portions of the attachment member which are connected to the enclosure and wherein the rest of the attachment member is inelastic. Such a structure can help to keep the enclosure and sensors close against the arm surface. This, in turn, can enable more-consistent collection of data concerning arm tissue.

In an example, the device in FIG. 30 can be specified as a wearable device for the arm with one or more close-fitting biometric sensors comprising: (a) an attachment member which is configured to span at least 60% of the circumference of a person's arm, wherein this attachment member further comprises—a first elastic portion with a first elasticity level, a second elastic portion with a second elasticity level, and an inelastic portion with a third elasticity level, wherein the third elasticity level is less than each of the first and second elasticity levels; (b) an enclosure which is connected between the first and second elastic portions; and (c) one or more biometric sensors which collect data concerning arm tissue which are part of (or attached to) the enclosure.

In an example, the device in FIG. 30 can be specified as a wearable device for the arm with one or more close-fitting biometric sensors comprising: (a) an attachment member which is configured to span at least 60% of the circumference of a person's arm, wherein this attachment member further comprises—a first plano-concave elastic portion with a first elasticity level, a second plano-concave elastic portion with a second elasticity level, and an inelastic portion with a third elasticity level, wherein the third elasticity level is less than each of the first and second elasticity levels; (b) an enclosure which is connected between the first and second plano-concave elastic portions; and (c) one or more biometric sensors which collect data concerning arm tissue which are part of (or attached to) the enclosure.

In an example, the device in FIG. 30 can be specified as a wearable device for the arm with one or more close-fitting biometric sensors comprising: (a) an enclosure which is configured to be worn on a person's arm; (b) one or more biometric sensors which collect data concerning arm tissue which are part of (or attached to) the enclosure; (c) two elastic members which are attached to the enclosure, wherein these elastic members are configured to collectively span at least 20% of the circumference of the arm, and wherein these elastic attachment members have first and second elasticity levels, respectively; and (d) one or more inelastic members which are attached to the two elastic attachment members, wherein these inelastic members collectively span at least 24% of the circumference of the arm, and wherein these inelastic members have a third elasticity level which is less than each of the first and second elasticity levels.

In an example, an elastic member can have a shape which is selected from the group consisting of: rectangular; rounded rectangle; plano-concave; and section of a cylinder. In an example, the device in FIG. 30 can be specified as a wearable device for the arm with one or more close-fitting biometric sensors comprising: (a) an attachment member which is configured to span at least 60% of the circumference of a person's arm, wherein this attachment member further comprises—two symmetric plano-concave elastic portions, with first and second elasticity levels respectively, and an inelastic portion with a third elasticity level, wherein the third elasticity level is less than each of the first and second elasticity levels; (b) an enclosure which is connected to the concave sides of the two symmetric plano-concave elastic portions of the attachment member; and (c) one or more biometric sensors which collect data concerning arm tissue which are part of (or attached to) the enclosure.

In an example, an attachment member can be a band, strap, bracelet, bangle, armlet, cuff, or sleeve. In an example, an elastic portion of an attachment member can be made from elastic and/or stretchable fabric. In an example, an enclosure can be arcuate. In an example, an enclosure can be circular. In an example, a device can further comprise a display screen on the outward-facing surface of an enclosure. In an example, a biometric sensor can be a spectroscopic sensor which is configured to measure the spectrum of light energy reflected from (and/or absorbed by) tissue of a person's arm. In an example, a biometric sensor can be an electromagnetic energy sensor which is configured to measure parameters and/or patterns of electromagnetic energy passing through (and/or emitted by) tissue of a person's arm. In an example, measured parameters and/or patterns of electromagnetic energy can be selected from the group consisting of: impedance, resistance, conductivity, and electromagnetic wave pattern.

With respect to specific components, the example shown in FIG. 30 includes: first elastic portion 3002 of an attachment member; second elastic portion 3003 of an attachment member; first inelastic portion 3001 of an attachment member; second inelastic portion 3004 of an attachment member; enclosure 3005 with a display screen on its outward-facing surface; and biometric sensors 3006 and 3007. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 31 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 31 is like the one shown in FIG. 30 except that in FIG. 31 the elastic portions are tapered (narrower in width) as they approach their connections with the enclosure.

With respect to specific components, the example shown in FIG. 31 includes: first tapered (width-varying) elastic portion 3102 of an attachment member; second tapered (width-varying) elastic portion 3103 of an attachment member; first inelastic portion 3101 of an attachment member; second inelastic portion 3104 of an attachment member; enclosure 3105 with a display screen on its outward-facing surface; and biometric sensors 3106 and 3107. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 32 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 32 is like the one shown in FIG. 30 except that in FIG. 32 there are four elastic portions, two connected to each side of the enclosure. Further, each elastic portion has a shape which is triangular and/or pennant shaped.

The example shown in FIG. 32 is a wearable device for the arm with one or more close-fitting biometric sensors comprising: (a) an attachment member which is configured to span at least 60% of the circumference of a person's arm, wherein this attachment member further comprises—four tapered (width-varying) elastic portions, wherein these elastic portions have a first elasticity level, and wherein these elastic portions are configured to be worn on the anterior (upper) portion of a person's arm; and one or more inelastic portions which comprise the remainder of the attachment member, wherein these inelastic portions have a second elasticity level which is less than the first elasticity level; (b) an enclosure, wherein a first side of the enclosure is connected to tapered ends of two of the four elastic portions of the attachment member and wherein a second (opposite) side of the enclosure is connected to tapered ends of the other two of the four elastic portions of the attachment member; and (c) one or more biometric sensors which collect data concerning arm tissue, wherein these sensors are part of (or attached to) the enclosure.

With respect to specific components, the example shown in FIG. 32 includes: first, second, third, and fourth tapered elastic portions (3202, 3203, 3204, and 3205) of an attachment member; first and second inelastic portions (3201 and 3206) of an attachment member; enclosure 3207 with a display screen on its outward-facing surface; and biometric sensors 3208 and 3209. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

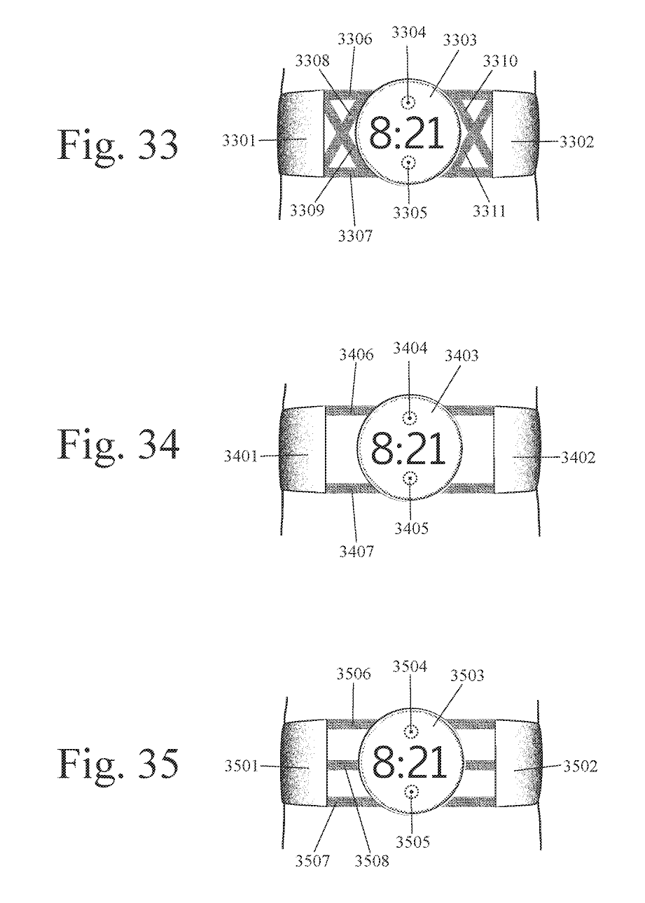

FIG. 33 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 33 is like the one shown in FIG. 30 except that in FIG. 33 there are two elastic portions on each side of the enclosure which criss-cross each other, forming an “X” on each side of the enclosure. This design can help to keep the enclosure and sensors close to the surface of the arm for more consistent collection of biometric data.

The example shown in FIG. 33 is a wearable device for the arm with one or more close-fitting biometric sensors comprising: (a) an enclosure which is configured to be worn on a person's arm; (b) one or more biometric sensors which collect data concerning arm tissue, wherein these sensors are part of (or attached to) the enclosure; and (c) an attachment member which is configured to attach the enclosure to the person's arm, wherein the attachment member is configured to span at least 60% of the circumference of the person's arm, wherein the portion of the attachment member that connects to the enclosure includes elastic bands (or straps) on each side of the enclosure which criss-cross each other. In an example, the criss-crossing bands (or straps) on each side of the enclosure form an “X” on each side of the enclosure.

With respect to specific components, the example shown in FIG. 33 includes: inelastic portion 3301 of the attachment member; inelastic portion 3302 of the attachment member; enclosure 3303 with an outward-facing display screen; biometric sensors 3304 and 3305; and elastic bands (or straps) 3306, 3307, 3308, 3309, 3310, and 3311. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 34 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 34 is like the one shown in FIG. 30 except that in FIG. 34 there are two parallel elastic bands (or straps) connected to the enclosure. This design can help to keep the enclosure and sensors close to the surface of the arm for more consistent collection of biometric data.

The example shown in FIG. 34 is a wearable device for the arm with one or more close-fitting biometric sensors comprising: (a) an enclosure which is configured to be worn on a person's arm; (b) one or more biometric sensors which collect data concerning arm tissue, wherein these sensors are part of (or attached to) the enclosure; and (c) an attachment member which is configured to attach the enclosure to the person's arm, wherein the attachment member is configured to span at least 60% of the circumference of the person's arm, and wherein the portion of the attachment member that connects to the enclosure includes two parallel elastic bands (or straps) connected to the enclosure.

With respect to specific components, the example shown in FIG. 34 includes: inelastic portion 3401 of the attachment member; inelastic portion 3402 of the attachment member; enclosure 3403 with an outward-facing display screen; biometric sensors 3404 and 3405; and two parallel elastic bands (or straps) 3406 and 3407. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 35 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 35 is like the one shown in FIG. 30 except that in FIG. 35 there are three parallel elastic bands (or straps) connected to the enclosure. This design can help to keep the enclosure and sensors close to the surface of the arm for more consistent collection of biometric data.

The example shown in FIG. 35 is a wearable device for the arm with one or more close-fitting biometric sensors comprising: (a) an enclosure which is configured to be worn on a person's arm; (b) one or more biometric sensors which collect data concerning arm tissue, wherein these sensors are part of (or attached to) the enclosure; and (c) an attachment member which is configured to attach the enclosure to the person's arm, wherein the attachment member is configured to span at least 60% of the circumference of the person's arm, and wherein the portion of the attachment member that connects to the enclosure includes three parallel elastic bands (or straps) connected to the enclosure.

With respect to specific components, the example shown in FIG. 35 includes: inelastic portion 3501 of the attachment member; inelastic portion 3502 of the attachment member; enclosure 3503 with an outward-facing display screen; biometric sensors 3504 and 3505; and three parallel elastic bands (or straps) 3506, 3507, and 3508. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

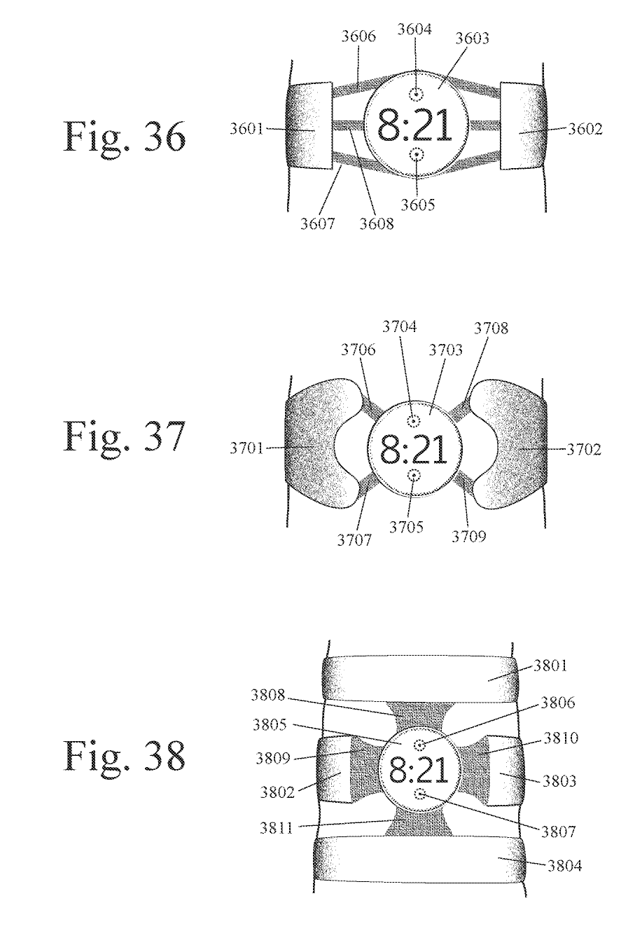

FIG. 36 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 36 is like the one shown in FIG. 35 except that the three elastic bands (or straps) are not parallel.

With respect to specific components, the example shown in FIG. 36 includes: inelastic portion 3601 of the attachment member; inelastic portion 3602 of the attachment member; enclosure 3603 with an outward-facing display screen; biometric sensors 3604 and 3605; and three elastic bands (or straps) 3606, 3607, and 3608. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 37 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 37 can be generally described as an arm-worn device with biometric sensors in an enclosure, wherein the enclosure is suspended on the surface of the arm by four elastic bands, and wherein each elastic band is individually connected to one of four points which are equally-spaced around the circumference of the enclosure. This enclosure suspension design can help to keep the sensors close to the surface of the arm for more consistent collection of biometric data.

The example shown in FIG. 37 can be specified as a wearable device for the arm with one or more close-fitting biometric sensors comprising: (a) an arcuate enclosure which is configured to be worn on a person's arm; (b) one or more biometric sensors which collect data concerning arm tissue, wherein these sensors are part of (or attached to) the enclosure; and (c) four elastic bands (or straps), each of which is connected to one of four points which are equally spaced around the circumference of the enclosure. In an example, each of the four elastic bands (or straps) can have one end which is connected to the enclosure and one end which is connected to an inelastic band, strap, bracelet, or armlet which is configured to span at least 50% of the circumference of the arm.

In another example, a wearable device for the arm with one or more close-fitting biometric sensors can comprise: (a) a quadrilateral enclosure which is configured to be worn on a person's arm; (b) one or more biometric sensors which collect data concerning arm tissue, wherein these sensors are part of (or attached to) the enclosure; and (c) four elastic bands (or straps), each of which is connected to one side of the enclosure. In an example, each of the four elastic bands (or straps) can have one end which is connected to the enclosure and one end which is connected to an inelastic band, strap, bracelet, or armlet which is configured to span at least 50% of the circumference of the arm.

With respect to specific components, the example shown in FIG. 37 includes: inelastic portion 3701 of the attachment member; inelastic portion 3702 of the attachment member; enclosure 3703 with an outward-facing display screen; biometric sensors 3704 and 3705; and four elastic bands (or straps) 3706, 3707, 3708, and 3709. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

FIG. 38 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 38 can be generally described as an arm-worn device with biometric sensors in an enclosure, wherein the enclosure is suspended on the surface of the arm by four elastic suspension bands (or straps) connected to three parallel attachment bands or straps which encircle the arm. This enclosure suspension design can help to keep the sensors close to the surface of the arm for more consistent collection of biometric data.

The example shown in FIG. 38 can be specified as a wearable device for the arm with one or more close-fitting biometric sensors comprising: (a) an enclosure which is configured to be worn on a person's arm; (b) one or more biometric sensors which collect data concerning arm tissue, wherein these sensors are part of (or attached to) the enclosure; and (c) three parallel attachment bands, including proximal, middle, distal attachment bands; wherein each of the three parallel attachment bands is configured to span at least 60% of the circumference of the arm; and (d) four elastic suspension bands, wherein the four suspension bands are connected to the four sides of the enclosure, respectively; wherein two of the suspension bands are also connected to the proximal attachment band and the distal attachment band, respectively; and wherein the other two of the suspension bands are also connected to the middle attachment band. In an example, the word “strap” can be substituted for the word “band” in the above specification.

With respect to specific components, the example shown in FIG. 38 includes: a distal attachment band 3801, ends 3802 and 3803 of a middle attachment band, proximal attachment band 3804, enclosure 3805 with outward-facing display screen, biometric sensors 3806 and 3807, and four suspension bands 3808, 3809, 3810, and 3811. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

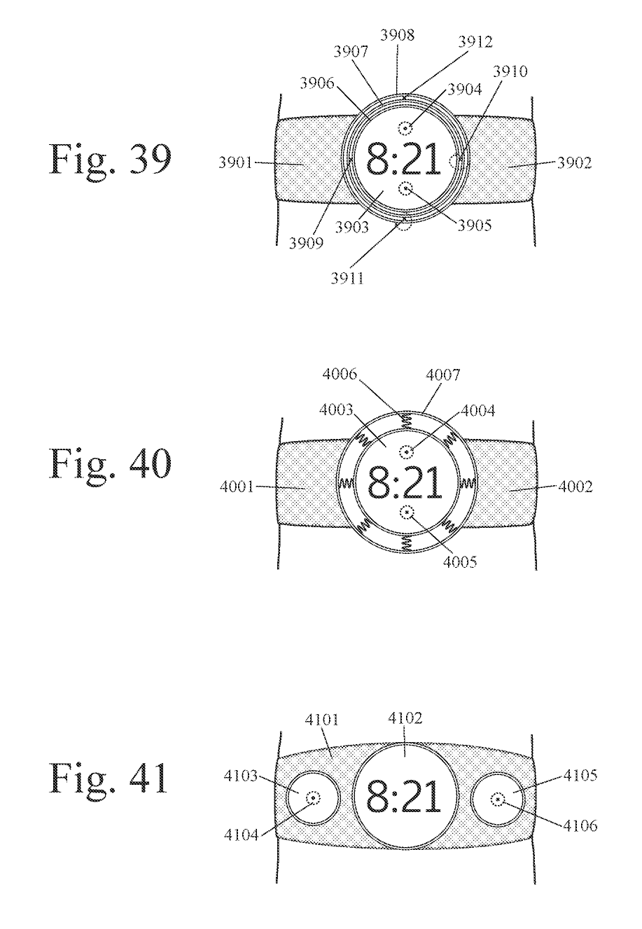

FIG. 39 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 39 can be described as a wrist-worn device with a gimbaled enclosure that contains one or more biometric sensors. The gimbal mechanism around the enclosure enables the enclosure and sensors to remain relatively flat against the surface of the arm, even if the device shifts, rotates, and/or twists on the person's arm. This can help to maintain consistent measurement of biometric data from the arm.

The example in FIG. 39 can be specified as a wearable device for the arm with one or more close-fitting biometric sensors comprising: (a) an attachment member which is configured to span at least 60% of the circumference of a person's arm; (b) a gimbal mechanism which is connected to the attachment member, wherein this gimbal mechanism further comprises two or more concentric rings which are axially connected so that they can move relative to each other; (c) an enclosure within the most central concentric ring of the gimbal mechanism; and (d) one or more biometric sensors which are part of (or attached to) the enclosure, wherein these biometric sensors are configured to collect data concerning arm tissue.

In an example, an attachment member can be a strap, band, bracelet, bangle, chain, ring, armlet, cuff, gauntlet, or sleeve. In an example, an attachment member can stretch to span the entire circumference of a person's arm. In an example, an attachment member can have two ends which connect to each other to hold the attachment member onto a person's arm. In an example, an attachment member can be sufficiently rigid and/or resilient in shape that it has ends which hold onto the person's arm even though the ends are not connected to each other.

In an example, a gimbal mechanism can comprise two concentric (inner and outer) rings which pivot relative to each other, wherein these rings are connected by one or more axles at opposite sides of the inner ring. In an example, a gimbal mechanism can comprise three concentric (inner, central, and outer) rings which pivot relative to each other, wherein the outer and central rings are connected by one or more axles at a first set of opposite sides of the central ring, wherein the central and inner rings are connected by one or more axles at a second set of opposite sides of the central ring, and wherein the second set is at locations which are rotated around the circumference of the center ring by 90-degrees relative to the locations of the first set.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, an enclosure can further comprise a display screen which is seen on the outward-facing surface of the enclosure. In an example, the enclosure can be circular. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

With respect to specific components, the example shown in FIG. 39 includes: first portion 3901 of an attachment member; second portion 3902 of the attachment member; enclosure 3903 with an outward-facing display screen; biometric sensors 3904 and 3905 within the enclosure; inner ring 3906, central ring 3907, and outer ring 3908 of a gimbal mechanism; first set of axles 3909 and 3910 connecting the inner ring and the central ring; and second set of axles 3911 and 3912 connecting the central ring and the outer ring.

FIG. 40 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 40 is a wrist-worn device with an enclosure containing one or more biometric sensors, wherein this enclosure is suspended by a radial plurality of elastic (and/or stretchable or springy) suspension members which connect to locations on the circumference of the enclosure. In an example, this design can be called a “sunburst suspension system” because the elastic (and/or stretchable or springy) suspension members look like the radial sunrays in a traditional “sunburst” drawing. The “sunburst suspension” design enables the enclosure and sensors to remain relatively flat against the surface of the arm, even if the device shifts, rotates, and/or twists on the person's arm. This can help to maintain consistent measurement of biometric data from the arm.

The example in FIG. 40 can be specified as a wearable device for the arm with one or more close-fitting biometric sensors comprising: (a) an attachment member which is configured to span at least 60% of the circumference of a person's arm; (b) an enclosure; (c) one or more biometric sensors which are part of (or attached to) the enclosure, wherein these biometric sensors are configured to collect data concerning arm tissue; and (d) a plurality of elastic (and/or stretchable or springy) suspension members, wherein these suspension members flexibly connect the enclosure to the attachment member, wherein each of these suspension members is connected at one end to a location on the circumference of the enclosure and connected at its other end to the attachment member, and wherein the longitudinal axis of each of the suspension members is substantially parallel with a virtual radial spoke outward from the center of the enclosure.

In an example, an attachment member can be a strap, band, bracelet, bangle, chain, ring, armlet, cuff, gauntlet, or sleeve. In an example, an attachment member can stretch to span the entire circumference of a person's arm. In an example, an attachment member can have two ends which connect to each other to hold the attachment member onto a person's arm. In an example, an attachment member can be sufficiently rigid and/or resilient in shape that it has ends which hold onto the person's arm, even though the ends of the attachment member are not connected to each other.

In an example, an enclosure can be circular. In an example, an enclosure can further comprise a display screen which is seen on the outward-facing surface of the enclosure. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, a suspension member can be a spring. In an example, a suspension member can be an elastic band or strap. In an example, the locations on the circumference of the enclosure to which the suspension members are connected can be evenly distributed around the circumference of the enclosure. In an example, there can be four suspension members. In an example, there can be six suspension members. In an example, there can be eight suspension members. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

With respect to specific components, the example shown in FIG. 40 includes: first portion 4001 of an attachment member; second portion 4002 of the attachment member; enclosure 4003 with an outward-facing display screen; biometric sensors 4004 and 4005 within the enclosure; a plurality of spring suspension members, including 4006; and ring 4007.

FIG. 41 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 41 is a flexible arm-worn device with two arcuate enclosures which contain biometric sensors.

The example in FIG. 41 can be specified as a wearable device for the arm with one or more close-fitting biometric sensors comprising: (a) a flexible attachment member which is configured to span at least 60% of the circumference of a person's arm; (b) a first arcuate enclosure whose center is at a first location on the circumference of the flexible attachment member; (c) a first biometric sensor which is part of (or attached to) the first arcuate enclosure, wherein this first biometric sensor is configured to collect data concerning arm tissue; (d) a second arcuate enclosure whose center is at a second location on the circumference of the flexible attachment member, wherein the distance between the first and second locations is greater than one-half inch; and (e) a second biometric sensor which is part of (or attached to) the second arcuate enclosure, wherein this second biometric sensor is configured to collect data concerning arm tissue.

In an example, a flexible attachment member can be a strap, band, bracelet, bangle, chain, ring, armlet, cuff, gauntlet, or sleeve. In an example, a flexible attachment member can stretch to span the entire circumference of a person's arm. In an example, a flexible attachment member can have two ends which connect to each other to hold the attachment member onto a person's arm. In an example, a flexible attachment member can be sufficiently rigid and/or resilient in shape that it has ends which hold onto the person's arm, even though the ends of the attachment member are not connected to each other.

In an example, an arcuate enclosure containing a biometric sensor can be circular. In an example, this device can further comprise a display screen between the two arcuate enclosures. In an alternative example, each of the arcuate enclosures can have a display screen on its outward-facing side. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 41 includes: flexible attachment member 4101; central display screen 4102; first arcuate enclosure 4103; first biometric sensor 4104; second arcuate enclosure 4105; and second biometric sensor 4106. In an example, this device can further comprise one or more components selected from the group consisting of: a data processor; a battery and/or energy harvesting unit; a display screen; a data transmitter; and a data receiver. In an example, relevant embodiment variations discussed elsewhere in this disclosure can also apply to this example.

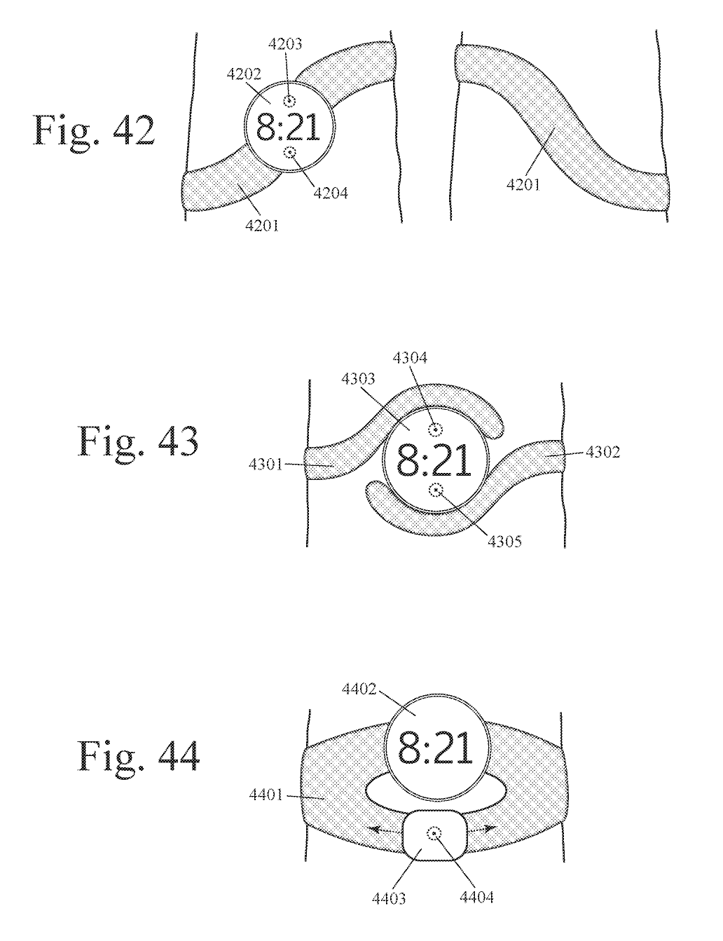

FIG. 42 shows another example of a wearable device for the arm with one or more close-fitting biometric sensors. The left side of this figure shows the device from a top-down perspective, as it would appear spanning the anterior (upper) surface of a person's wrist (or other portion of the person's arm) in a circumferential manner. The right side of this figure shows the device from a bottom-up perspective, as it would appear spanning the posterior (lower) surface of a person's wrist (or other portion of the person's arm) in a circumferential manner. The example shown in FIG. 42 can be described as an arm-worn device with an arcuate enclosure to which a strap or band is connected diagonally.

The example in FIG. 42 can be specified as a wearable device for the arm with one or more close-fitting biometric sensors comprising: (a) an arcuate enclosure which is configured to be worn on a person's arm; (b) one or more biometric sensors which are configured to collect data concerning arm tissue and which are part of (or attached to) the arcuate enclosure; and (c) an attachment member which is configured to span at least 60% of the circumference of a person's arm, wherein a first portion of this attachment member is connected to the arcuate enclosure between the 12 o'clock and 3 o'clock positions (or 0-degree and 90-degree positions using compass coordinates) of the circumference of the enclosure; and wherein a second portion of this attachment member is connected to the arcuate enclosure between the 6 o'clock and 9 o'clock positions (or 180-degree and 270-degree positions using compass coordinates) of the circumference of the enclosure.

With respect to specific components, the example shown in FIG. 42 includes: attachment member 4201; enclosure 4202 with an outward-facing display screen; and biometric sensors 4203 and 4204 within the enclosure. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

FIG. 43 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 43 can be colorfully described as a “two gummi worm” design—because it looks like two gummi worms crawling in a symmetric manner around portions of the circumference of an enclosure.

The example in FIG. 43 can be specified as a wearable device for the arm with one or more close-fitting biometric sensors comprising: (a) an arcuate enclosure which is configured to be worn on a person's arm; (b) one or more biometric sensors which are configured to collect data concerning arm tissue and which are part of (or attached to) the arcuate enclosure; and (c) an attachment member which is configured to span at least 60% of the circumference of a person's arm, wherein a first portion of this attachment member spans most of the circumference of the arcuate enclosure between the 10 o'clock and 2 o'clock positions (or 300-degree and 60-degree positions using compass coordinates); and wherein a second portion of this attachment member spans most of the circumference of the arcuate enclosure between the 4 o'clock and 8 o'clock positions (or 120-degree and 240-degree positions using compass coordinates).

With respect to specific components, the example shown in FIG. 43 includes: first portion 4301 of an attachment member; second portion 4302 of an attachment member; enclosure 4303 with an outward-facing display screen; and biometric sensors 4304 and 4305 within the enclosure. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

FIG. 44 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 44 can be generally described as an arm-worn device with a bifurcating band, wherein one branch of the band is connected to a display screen and the other branch of the band is connected to a circumferentially-sliding enclosure which contains one or more biometric sensors. Having biometric sensors on a separate circumferentially-sliding enclosure enables adjustment of the circumferential location from which biometric data is collected, without changing the location of a display screen.

The example in FIG. 44 can be specified as a wearable device for the arm with one or more close-fitting biometric sensors comprising: (a) an attachment member which is configured to span at least 60% of the circumference of a person's arm, wherein this attachment member has a first circumferential portion which bifurcates into a first branch and a second branch, and wherein this attachment member has a second circumferential portion in which the first branch and the second branch reconverge; (b) a display screen which is connected to the first branch of the attachment member; (c) a circumferentially-sliding enclosure which is connected to the second branch of the attachment member; and (d) one or more biometric sensors which are configured to collect data concerning arm tissue and which are part of (or attached to) the circumferentially-sliding enclosure.

With respect to specific components, the example shown in FIG. 44 includes: bifurcating attachment member 4401; display screen 4402 on a first branch of the attachment member; circumferentially-sliding enclosure 4403 on a second branch of the attachment member; and biometric sensor 4404 within the circumferentially-sliding enclosure. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

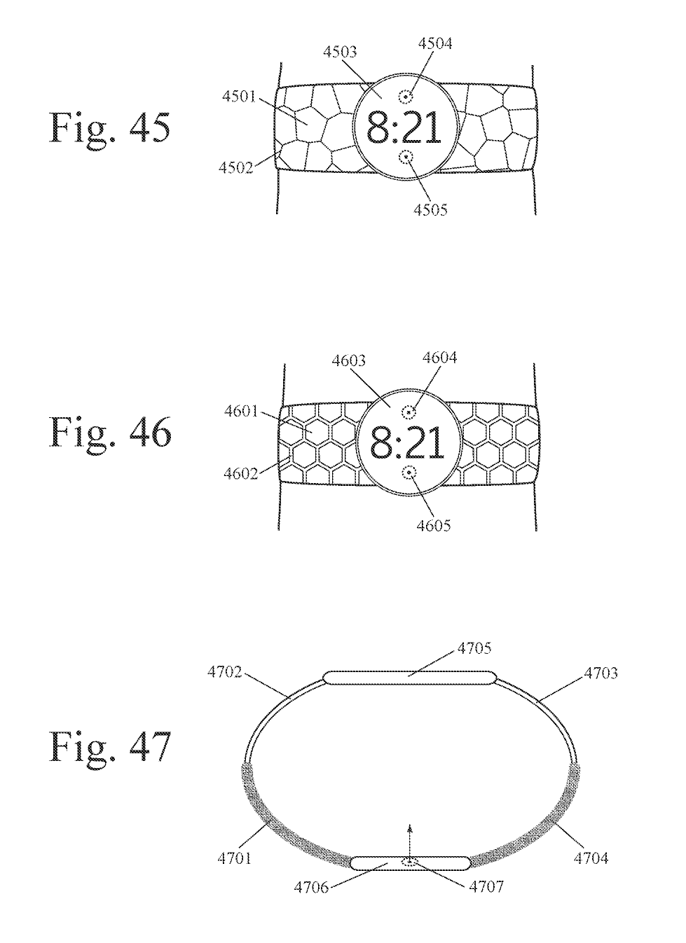

FIG. 45 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner.

The example in FIG. 45 can be specified as a wearable device for the arm with one or more close-fitting biometric sensors comprising: (a) an attachment member which is configured to span at least 60% of the circumference of a person's arm, wherein this attachment member further comprises a plurality of various-shaped (rigid) polygons which are inter-connected by flexible strips and/or joints; (b) an enclosure which is connected to the attachment member; and (c) one or more biometric sensors which are configured to collect data concerning arm tissue and which are part of (or attached to) the enclosure. Such a design can help to keep the enclosure (and thus the sensors) flat against the surface of the person's arm, even if the attachment member shifts, twists, or rotates.

In an example, a majority of the various-shaped polygons can have five sides. In an example, a majority of the various-shaped polygons can have six sides. In an example, a majority of the various-shaped polygons can have unequal sides. In an example, a majority of the various-shaped polygons can have unequal angles between sides. In an example, sides of the various-shaped polygons can be inter-connected by strips of flexible fabric. In an example, sides of the various-shaped polygons can be inter-connected by hinge joints. In an example, the enclosure can have a display screen on its outward-facing surface.

With respect to specific components, the example shown in FIG. 45 includes: a plurality of various-shaped inter-connected polygons, including polygon 4501; a plurality of flexible joints, including joint 4502; an arcuate enclosure 4503 which further comprises a display screen on its outward-facing surface; and biometric sensors 4504 and 4505 within the enclosure. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

FIG. 46 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. In this example, an attachment member has a honeycomb configuration. This can help to keep the enclosure (and thus the sensors) flat against the surface of the person's arm, even if the attachment member shifts, twists, or rotates.

The example in FIG. 46 can be specified as a wearable device for the arm with one or more close-fitting biometric sensors comprising: (a) an attachment member which is configured to span at least 60% of the circumference of a person's arm, wherein this attachment member further comprises a plurality of flexibly-connected (rigid) hexagons; (b) an enclosure which is connected to the attachment member; and (c) one or more biometric sensors which are configured to collect data concerning arm tissue and which are part of (or attached to) the enclosure.

With respect to specific components, the example shown in FIG. 46 includes: a plurality of hexagons, including hexagon 4601; a plurality of flexible joints, including joint 4602; an arcuate enclosure 4603 which further comprises a display screen on its outward-facing surface; and biometric sensors 4604 and 4605 within the enclosure. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

FIG. 47 shows another example of a wearable device for the arm with one or more close-fitting biometric sensors. This figure shows the device from a side perspective, as it would appear encircling a lateral cross-section of a person's wrist (or other portion of the person's arm). In this example, one or more biometric sensors are located on a portion of the device which is diametrically-opposite (e.g. symmetric relative to the circumferential center of the device) from the portion of the device which includes a display screen. In an example, one or more biometric sensors can be configured to be worn on the posterior (lower) surface of an arm and a display screen can be configured to be worn on the anterior (upper) surface of the arm, or vice versa.

The specific example in FIG. 47 can be specified as a wearable device for the arm with one or more close-fitting biometric sensors comprising: (a) an attachment member which is configured to span at least 60% of the circumference of a person's arm, wherein this attachment member further comprises a first portion with a first elasticity level spanning completely or partially (clockwise) between the 9 o'clock and 3 o'clock (or 270-degree and 90-degree) positions around the device circumference and a second portion with a second elasticity level spanning completely or partially (clockwise) between the 3 o'clock and 9 o'clock (or 90-degree and 270-degree) positions around the device circumference, wherein the second elasticity level is greater than the first elasticity level; (b) a display screen which is part of (or connected to) the first portion of the attachment member; (c) an enclosure which is part of (or connected to) the second portion of the attachment member; and (d) one or more biometric sensors which are configured to collect data concerning arm tissue and which are part of (or attached to) the enclosure.

In an example, the display screen can be centrally located with respect to the first portion of the attachment member. In an example, the center of the display screen can be located at the 12 o'clock (or 0-degrees) position on the circumference of the device. In an example, the enclosure can be centrally located with respect to the second portion of the attachment member. In an example, the center of the display screen can be located at the 6 o'clock (or 180-degrees) position on the circumference of the device.

With respect to specific components, the example shown in FIG. 47 includes: elastic segments 4701 and 4704 of an attachment member; inelastic segments 4702 and 4703 of an attachment member; display screen 4705; enclosure 4706; and biometric sensor 4707. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

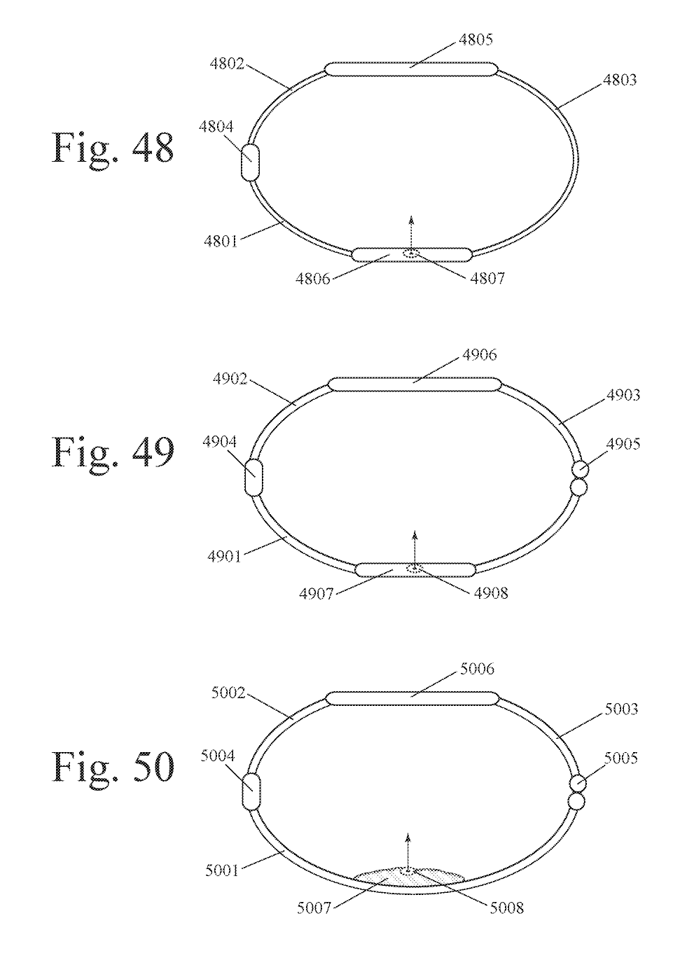

FIG. 48 shows another example of a wearable device for the arm with one or more close-fitting biometric sensors. This figure shows the device from a side perspective, as it would appear encircling a lateral cross-section of a person's wrist (or other portion of the person's arm). In this example, one or more biometric sensors are located on a portion of the device which is diametrically-opposite (e.g. symmetric relative to the circumferential center of the device) from the portion of the device which includes a display screen and there is a connector (such as a buckle, clip, clasp, pin, plug, or hook-and-eye mechanism) on the device between the sensors and the screen.

The example in FIG. 48 can be specified as a wearable device for the arm with one or more close-fitting biometric sensors comprising: (a) an attachment member which is configured to span at least 60% of the circumference of a person's arm; (b) a display screen which is part of (or connected to) the attachment member at a first location along the circumference of the device; (c) an enclosure which is part of (or connected to) the attachment member at a second location along the circumference of the device, wherein the second location is on the opposite side of the device (e.g. through the circumferential center of the device) from the first location; (d) one or more biometric sensors which are configured to collect data concerning arm tissue and which are part of (or attached to) the enclosure; and (e) a connector which connects two ends of the attachment member to each other, wherein this connector is at a location along the circumference of the device which is between the display screen and the enclosure.

The example in FIG. 48 can also be specified as a wearable device for the arm with one or more close-fitting biometric sensors comprising: (a) an attachment member which is configured to span at least 60% of the circumference of a person's arm; (b) a display screen which is located between the 10 o'clock and 2 o'clock (or 300-degree and 60-degree) positions on the circumference of the attachment member; (c) an enclosure which is located between the 4 o'clock and 8 o'clock (or 120-degree and 240-degree) positions on the circumference of the attachment member; (d) one or more biometric sensors which are configured to collect data concerning arm tissue and which are part of (or attached to) the enclosure; and (e) a connector which connects two ends of the attachment member to each other, wherein this connector is at a location along the circumference of the device which is between the display screen and the enclosure.

In an example, the center of the display screen can be located at the 12 o'clock (or 0-degrees) position on the circumference of the device. In an example, the center of the display screen can be located at the 6 o'clock (or 180-degrees) position on the circumference of the device. In an example, a connector can be selected from the group consisting of: buckle, clip, clasp, hook, plug, pin, snap, and hook-and-eye mechanism.

With respect to specific components, the example shown in FIG. 48 includes: segments 4801, 4802, and 4803 of an attachment member; connector 4804; display screen 4805; enclosure 4806; and biometric sensor 4807. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

FIG. 49 shows another example of a wearable device for the arm with one or more close-fitting biometric sensors. This figure shows the device from a side perspective, as it would appear encircling a lateral cross-section of a person's wrist (or other portion of the person's arm). In this example, there are one or more biometric sensors which are opposite a display screen, a connector between the sensors and the screen, and a hinge which is opposite the connector. If portions of an attachment member connecting these components are relatively rigid, then this example can be called a “clam shell” design.

The example in FIG. 49 can be specified as a wearable device for the arm with one or more close-fitting biometric sensors comprising: (a) an attachment member which is configured to span at least 60% of the circumference of a person's arm; (b) a display screen which is part of (or connected to) the attachment member at a first location around the circumference of the device; (c) an enclosure which is part of (or connected to) the attachment member at a second location around the circumference of the device, wherein the second location is on the opposite (e.g. through the circumferential center) side of the device from the first location; (d) one or more biometric sensors which are configured to collect data concerning arm tissue and which are part of (or attached to) the enclosure; and (e) a connector which connects two ends of the attachment member to each other at a third location around the circumference of the device, wherein this third location is between the first and second locations; (f) a hinge (or joint) which connects two portions of the attachment member to each other at a fourth location around the circumference of the device, wherein this fourth location is on the opposite (e.g. through the circumferential center) side of the device from the third location.

The example in FIG. 49 can also be specified as a wearable device for the arm with one or more close-fitting biometric sensors comprising: (a) an attachment member which is configured to span at least 60% of the circumference of a person's arm; (b) a display screen which is located between the 10 o'clock and 2 o'clock (or 300-degree and 60-degree) positions on the circumference of the attachment member; (c) an enclosure which is located between the 4 o'clock and 8 o'clock (or 120-degree and 240-degree) positions on the circumference of the attachment member; (d) one or more biometric sensors which are configured to collect data concerning arm tissue and which are part of (or attached to) the enclosure; (e) a connector which connects two ends of the attachment member to each other, wherein this connector is located between the 7 o'clock and 11 o'clock (or 210-degree and 330-degree) positions on the circumference of the attachment member; and (f) a hinge which connects two portions of the attachment member to each other, wherein this hinge is located between the 1 o'clock and 5 o'clock (or 30-degree and 150-degree) positions on the circumference of the attachment member.

In an example, the center of the display screen can be located at the 12 o'clock (or 0-degrees) position on the circumference of the device. In an example, the center of the display screen can be located at the 6 o'clock (or 180-degrees) position on the circumference of the device. In an example, a connector can be selected from the group consisting of: buckle, clip, clasp, hook, plug, pin, snap, and hook-and-eye mechanism.

With respect to specific components, the example shown in FIG. 49 includes: segments 4901, 4902, and 4903 of an attachment member; connector 4904; hinge 4905; display screen 4906; enclosure 4907; and biometric sensor 4908. In an example, this device can further comprise one or more components selected from the group consisting of: a data processor; a battery and/or energy harvesting unit; a display screen; a data transmitter; and a data receiver. Relevant embodiment variations discussed with respect to FIG. 39 or elsewhere in this disclosure can also be applied to this example.

FIG. 50 shows another example of a wearable device for the arm with one or more close-fitting biometric sensors. This figure shows the device from a side perspective, as it would appear encircling a lateral cross-section of a person's wrist (or other portion of the person's arm). This example is similar to the one shown in FIG. 49 except a biometric sensor is on the center-facing surface of a compressible member.

With respect to specific components, the example shown in FIG. 50 includes: segments 5001, 5002, and 5003 of an attachment member; connector 5004; hinge 5005; display screen 5006; compressible member 5007; and biometric sensor 5008. In an example, this device can further comprise one or more components selected from the group consisting of: a data processor; a battery and/or energy harvesting unit; a display screen; a data transmitter; and a data receiver.

In an example, a compressible member can be an elastic member which is filled with a fluid, gel, or gas. In an example, a compressible member can be a pneumatic or hydraulic chamber which is filled with a fluid, gel, or gas. In an example, a compressible member can be a balloon. In an example, a compressible member can be made from compressible foam. Relevant embodiment variations discussed with respect to FIG. 49 or elsewhere in this disclosure can also be applied to this example.

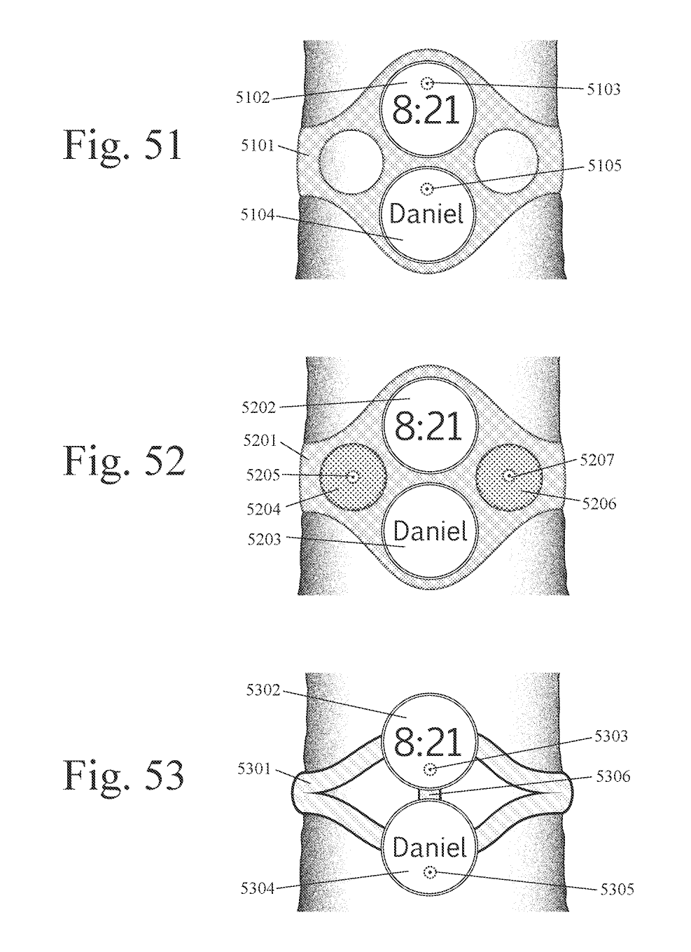

FIG. 51 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. Described generally, the example shown in FIG. 51 is a biometric-sensing device with proximal and distal arcuate display screens which are attached to a person's arm by a band, wherein the band has one holes on each side of a virtual line connecting the centers of the two displays.

Described more specifically, the example shown in FIG. 51 is a wearable device for the arm with one or more close-fitting biometric sensors comprising: (a) a proximal arcuate display screen, wherein this proximal arcuate display screen is a first distance from a person's shoulder; (b) a distal arcuate display screen, wherein this distal arcuate display screen is a second distance from a person's shoulder, and wherein the second distance is greater than the first distance; (c) an attachment member which attaches the proximal arcuate display screen and the distal arcuate display screen to the person's arm, wherein this attachment member has one hole on each side of a virtual line connecting the center of the proximal arcuate display screen and the center of the distal arcuate display screen; and (d) one or more biometric sensors which are configured to collect data concerning arm tissue.

In an example, a display screen can be circular. In an example, a display screen can be activated by touch and/or gesture. In an example, a virtual line connecting the center of a proximal display screen and the center of a distal display screen can parallel to the longitudinal axis of the arm. In an example, holes on each side of this virtual line can be circular. In an example, the area of a hole in an attachment member can be half of the area of a display screen. In an example, the area of a hole in an attachment member can be the same as the area of a display screen. In an example, the area of a hole in an attachment member can be between 50% and 100% of the area of a display screen.

In an example, an attachment member can be a strap, band, bracelet, bangle, ring, armlet, gauntlet, cuff, or sleeve. In an example, an attachment member can be wider as it spans the anterior (upper) surface of a person's arm. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

Specific components in the example shown in FIG. 51 include: attachment member 5101 which has a hole on each side of a central longitudinal axis of the anterior (upper) surface of an arm; distal display screen 5102; proximal display screen 5104; and biometric sensors 5103 and 5105. In an example, this device can further comprise one or more components selected from the group consisting of: a data processor; a battery and/or energy harvesting unit; a data transmitter; and a data receiver. In an example, this device can function as a smart watch. Relevant embodiment variations discussed elsewhere in this disclosure can also be applied to this example.

FIG. 52 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. Described generally, the example shown in FIG. 52 is an arm-worn biometric-sensing device with: proximal and distal arcuate display screens; and right and left side enclosures with biometric sensors.

Described more specifically, the example shown in FIG. 52 is a wearable device for the arm with one or more close-fitting biometric sensors comprising: (a) a proximal arcuate display screen, wherein this proximal arcuate display screen is a first distance from a person's shoulder; (b) a distal arcuate display screen, wherein this distal arcuate display screen is a second distance from a person's shoulder, and wherein the second distance is greater than the first distance; (c) a right-side enclosure, wherein the center of this right-side enclosure is to the right of a virtual line connecting the center of the proximal arcuate display screen and the center of the distal arcuate display screen, and wherein this right-side enclosure further comprises a biometric sensor that is configured to collect data concerning arm tissue; (d) a left-side enclosure, wherein the center of this left-side enclosure is to the left of a virtual line connecting the center of the proximal arcuate display screen and the center of the distal arcuate display screen, and wherein this left-side enclosure further comprises a biometric sensor that is configured to collect data concerning arm tissue; and (e) an attachment member which attaches the proximal arcuate display screen, the distal arcuate display screen, the right-side enclosure, and the left-side enclosure to the person's arm.

In an example, a display screen can be circular. In an example, a display screen can be activated by touch and/or gesture. In an example, a virtual line connecting the center of a proximal display screen and the center of a distal display screen can parallel to the longitudinal axis of the arm. In an example, an attachment member can be a strap, band, bracelet, bangle, ring, armlet, gauntlet, cuff, or sleeve. In an example, an attachment member can be wider as it spans the anterior (upper) surface of a person's arm. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

Specific components in the example shown in FIG. 52 include: attachment member 5201; distal display screen 5202; proximal display screen 5203; right-side enclosure 5206 with biometric sensor 5207; and left-side enclosure 5204 with biometric sensor 5205. In an example, this device can further comprise one or more components selected from the group consisting of: a data processor; a battery and/or energy harvesting unit; a data transmitter; and a data receiver. In an example, this device can function as a smart watch. Relevant embodiment variations discussed elsewhere in this disclosure can also be applied to this example.

FIG. 53 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. Described generally, the example shown in FIG. 53 is an arm-worn biometric-sensing device with proximal and distal arcuate display screens which are circumferentially attached to an arm by a bifurcating band (or strap) and also connected to each other by a band (or strap) along the central longitudinal axis of the anterior (upper) surface of the arm.

Described more specifically, the example shown in FIG. 53 is a wearable device for the arm with one or more close-fitting biometric sensors comprising: (a) a proximal arcuate display screen, wherein this proximal arcuate display screen is configured to be worn a first distance from a person's shoulder; (b) a distal arcuate display screen, wherein this distal arcuate display screen is configured to be worn a second distance from a person's shoulder, and wherein the second distance is greater than the first distance; (c) one or more biometric sensors that are configured to collect data concerning arm tissue; (d) a bifurcated attachment member, wherein this bifurcated attachment member bifurcates into a proximal and distal branches as it spans the anterior (upper) surface of the person's arm, wherein these proximal and distal branches reconverge as the bifurcated attachment member further spans the anterior (upper) surface of the person's arm, wherein the proximal branch is configured to attach the proximal arcuate display screen to the person's arm, and wherein the distal branch is configured to attach the distal arcuate display screen to the person's arm; and (e) an inter-display connecting band (or strip) which connects the proximal arcuate display screen to the distal arcuate display screen.

In an example, a display screen can be circular. In an example, a display screen can be activated by touch and/or gesture. In an example, a virtual line connecting the center of a proximal display screen and the center of a distal display screen can parallel to the longitudinal axis of the arm. In an example, an inter-display connecting band (or strip) connecting the center of a proximal display screen and the center of a distal display screen can parallel to the longitudinal axis of the arm.

In an example, an attachment member can be a strap, band, bracelet, bangle, ring, armlet, gauntlet, cuff, or sleeve. In an example, an attachment member can be wider as it spans the anterior (upper) surface of a person's arm. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

Specific components in the example shown in FIG. 53 include: bifurcated attachment member 5301; distal display screen 5302; proximal display screen 5304; biometric sensors 5303 and 5305; and inter-display connecting band 5306. In an example, this device can further comprise one or more components selected from the group consisting of: a data processor; a battery and/or energy harvesting unit; a data transmitter; and a data receiver. In an example, this device can function as a smart watch. Relevant embodiment variations discussed elsewhere in this disclosure can also be applied to this example.

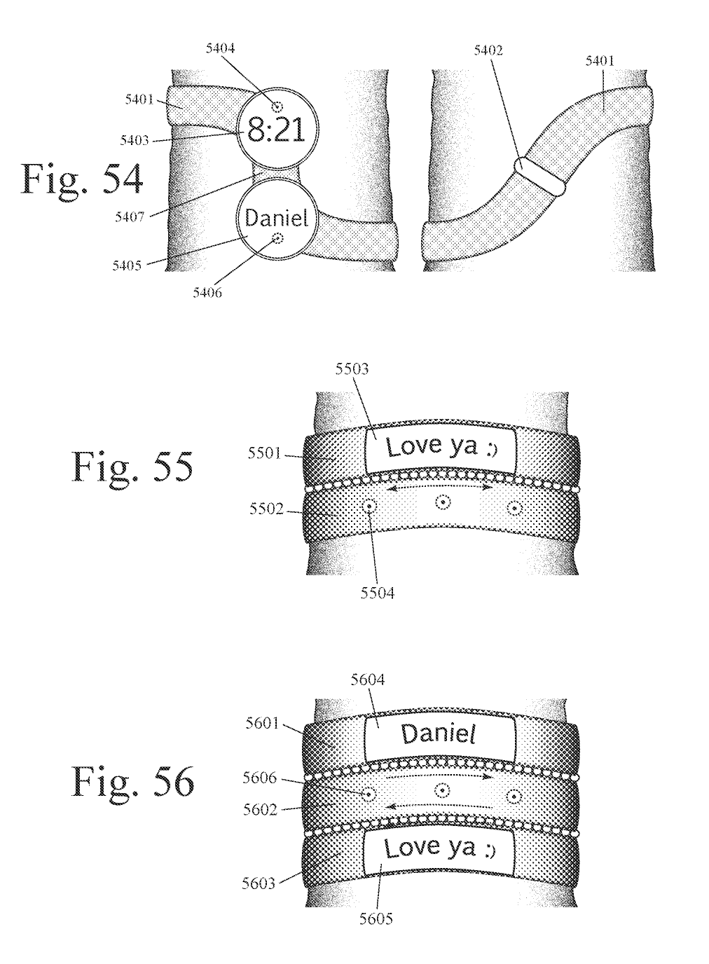

FIG. 54 shows another example of a wearable device for the arm with one or more close-fitting biometric sensors. The left side of this figure shows the device from a top-down perspective, as it would appear spanning the anterior (upper) surface of a person's wrist (or other portion of the person's arm) in a circumferential manner. The right side of this figure shows the device from a bottom-up perspective, as it would appear spanning the posterior (lower) surface of a person's wrist (or other portion of the person's arm) in a circumferential manner. Described generally, the example shown in FIG. 54 is an arm-worn biometric-sensing device with proximal and distal arcuate display screens which are attached to a person's arm by a band with an “S”-shaped portion spanning the anterior (upper) portion of the arm.

Described more specifically, the example shown in FIG. 54 is a wearable device for the arm with one or more close-fitting biometric sensors comprising: (a) a proximal arcuate display screen, wherein this proximal arcuate display screen is configured to be worn a first distance from a person's shoulder; (b) a distal arcuate display screen, wherein this distal arcuate display screen is configured to be worn a second distance from a person's shoulder, and wherein the second distance is greater than the first distance; (c) one or more biometric sensors that are configured to collect data concerning arm tissue; (d) a attachment member which is attached to the right side of the proximal arcuate display screen and to the left side of the distal arcuate display screen; (e) an inter-display connecting band which connects the distal portion of the proximal display screen to the proximal portion of the distal arcuate display screen.

Alternatively, a wearable device for the arm with one or more close-fitting biometric sensors can comprise: (a) a proximal arcuate display screen, wherein this proximal arcuate display screen is configured to be worn a first distance from a person's shoulder; (b) a distal arcuate display screen, wherein this distal arcuate display screen is configured to be worn a second distance from a person's shoulder, and wherein the second distance is greater than the first distance; (c) one or more biometric sensors that are configured to collect data concerning arm tissue; (d) a attachment member which is attached to the left side of the proximal arcuate display screen and to the right side of the distal arcuate display screen; (e) an inter-display band which connects the distal portion of the proximal display screen to the proximal portion of the distal arcuate display screen.

In an example, an attachment member can be a strap, band, bracelet, bangle, ring, armlet, gauntlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

Specific components in the example shown in FIG. 54 include: attachment member 5401; connector 5402; distal display screen 5403; proximal display screen 5405; biometric sensors 5404 and 5406; and inter-display band 5407. In an example, this device can further comprise one or more components selected from the group consisting of: a data processor; a battery and/or energy harvesting unit; a data transmitter; and a data receiver. In an example, this device can function as a smart watch. Relevant embodiment variations discussed elsewhere in this disclosure can also be applied to this example.

FIG. 55 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 55 can be described as a wearable device with two bands which encircle an arm, wherein these two bands are movably-attached to each other in a manner which allows a second band (with biometric sensors) to be rotated relative to a first band. Such rotation enables adjustment of the locations of one or more biometric sensors relative to the arm in order to improve collection of biometric data from arm tissue.

More specifically, the example shown in FIG. 55 is a wearable device for the arm with one or more close-fitting biometric sensors comprising: (a) a first band which is configured to span at least 60% of the circumference of a person's arm; (b) a second band which is configured to span at least 60% of the circumference of the person's arm, wherein the first band and the second band are attached to each other by a mechanism that enables the second band to be circumferentially-rotated relative to the first band; and (c) one or more biometric sensors which are configured to collect data concerning arm tissue, wherein these biometric sensors are part of (or attached to) the second band.

More generally, the example shown in FIG. 55 is a wearable device for the arm with one or more close-fitting biometric sensors comprising: (a) a first attachment member which is configured to span at least 60% of the circumference of a person's arm; (b) a second attachment member which is configured to span at least 60% of the circumference of the person's arm, wherein the first attachment member and the second attachment member are attached to each other by a mechanism that enables the second attachment member to be circumferentially-rotated relative to the first attachment member; and (c) one or more biometric sensors which are configured to collect data concerning arm tissue, wherein these biometric sensors are part of (or attached to) the second attachment member.

In an example, an attachment member can be a band, ring, strap, bracelet, bangle, or armlet. In an example, a band or other attachment member can be attached to a person's arm by connecting two ends of the attachment member with a clasp, clip, buckle, hook, pin, plug, or hook-and-eye mechanism. In an example, a band or other attachment member can be attached to a person's arm by stretching and sliding it over the person's hand onto the arm. In an example, a band or other attachment member can be attached to a person's arm by applying force to pull two ends apart to slip the member over the arm, wherein the two ends retract back towards each other when the force is removed.

In an example, a first attachment member can be attached to a person's arm in a relatively-fixed manner, so that it does not substantively rotate and/or shift around the circumference of the arm. In an example, a second attachment member can be attached to a person's arm in a relatively-loose manner, so that it can rotate around the circumference of the arm. In an example, a second attachment member can be attached (or connected) to the first attachment member by a connection mechanism which enables the second attachment member to be rotated around the circumference of the person's arm (relative to the first attachment member).

When the second attachment member contains one or more biometric sensors, rotation of the second attachment member also rotates these sensors relative to the circumference of the arm. This enables a user to find the optimal locations around the circumference of the arm from which to collect biometric data for a selected application. In an example, this device can further include a locking mechanism which locks the location of the second attachment member relative to the first attachment member when the optimal location for sensors is found. In an example, a connection mechanism between the two attachment members can be a ball-bearing mechanism. In an example, a connection mechanism can be a sliding tongue-and-groove (or tongue-and-slot) mechanism.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, a first attachment member can include a display screen on its outward-facing surface. In an example, this device can function as a smart watch. Relevant embodiment variations discussed elsewhere in this disclosure can also be applied to this example. Specific components in the example shown in FIG. 55 include: first band 5501; second band 5502; display screen 5503; and biometric sensors including 5504.

FIG. 56 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 56 is like the one shown in FIG. 55, except that in FIG. 56 there are three bands instead of two and biometric sensors are on a central band which rotates relative to distal and proximal bands. Such rotation enables adjustment of the locations of one or more biometric sensors relative to the arm in order to improve collection of biometric data from arm tissue.

The example shown in FIG. 56 is a wearable device for the arm with one or more close-fitting biometric sensors comprising: (a) a distal attachment member which is configured to span at least 60% of the circumference of a person's arm; (b) a proximal attachment member which is configured to span at least 60% of the circumference of a person's arm; (c) a central attachment member which is configured to span at least 60% of the circumference of the person's arm, wherein this central attachment member is between the distal and proximal attachment members, and wherein this central attachment member is circumferentially-rotated relative to the distal and proximal attachment members; and (d) one or more biometric sensors which are configured to collect data concerning arm tissue, wherein these biometric sensors are part of (or attached to) the central attachment member.

In an example, an attachment member can be a band, ring, strap, bracelet, bangle, or armlet. In an example, a band or other attachment member can be attached to a person's arm by connecting two ends of the attachment member with a clasp, clip, buckle, hook, pin, plug, or hook-and-eye mechanism. In an example, a band or other attachment member can be attached to a person's arm by stretching and sliding it over the person's hand onto the arm. In an example, a band or other attachment member can be attached to a person's arm by applying force to pull two ends apart to slip the member over the arm, wherein the two ends retract back towards each other when the force is removed.

In an example, distal and proximal attachment members can be attached to a person's arm in a relatively-fixed manner, so that they do not substantively rotate and/or shift around the circumference of the arm. In an example, a central attachment member can be attached to a person's arm in a relatively-loose manner, so that it can rotate around the circumference of the arm. In an example, a central attachment member can be attached (or connected) to the distal and proximal attachment members by a connection mechanism which enables the second attachment member to be rotated around the circumference of the person's arm.

When a central attachment member contains one or more biometric sensors, rotation of the central attachment member also rotates these sensors relative to the circumference of the arm. This enables a user to find the optimal locations around the circumference of the arm from which to collect biometric data for a selected application. In an example, this device can further include a locking mechanism which locks the location of the central attachment member relative to the distal and proximal attachment members when the optimal location for sensors is found. In an example, a connection mechanism between the two attachment members can be a ball-bearing mechanism. In an example, a connection mechanism can be a sliding tongue-and-groove (or tongue-and-slot) mechanism.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, a distal and/or proximal attachment member can include a display screen on an outward-facing surface. In an example, this device can function as a smart watch. Relevant embodiment variations discussed elsewhere in this disclosure can also be applied to this example. Specific components in the example shown in FIG. 56 include: distal band 5601; central band 5602; proximal band 5603; display screens 5604 and 5605; and biometric sensors including 5606.

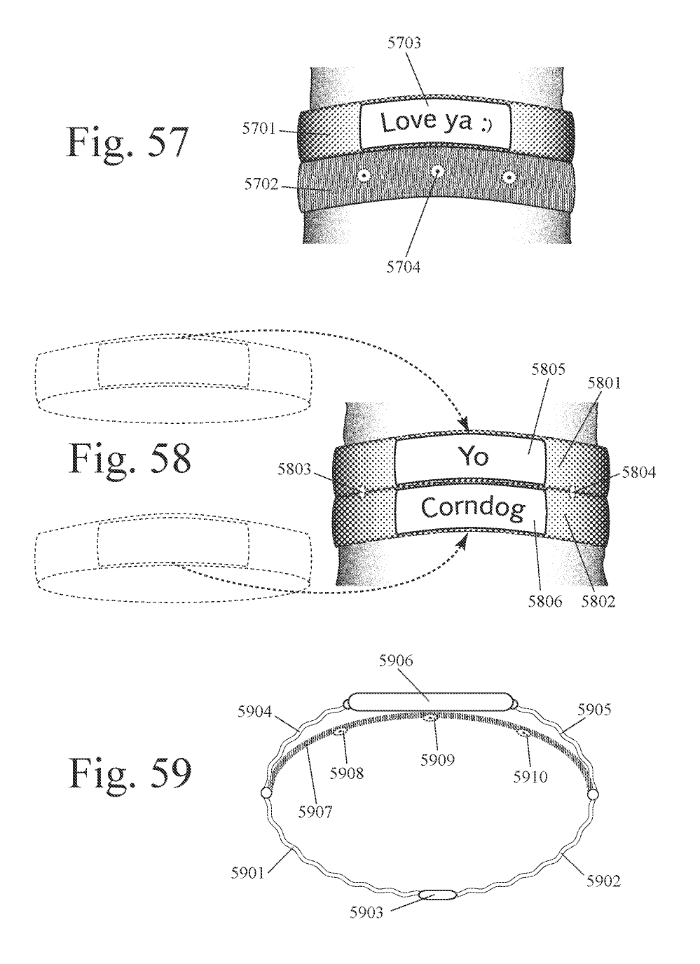

FIG. 57 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 57 can be described as an arm-wearable device with a relatively-rigid band and a relatively-elastic band, wherein each of these bands spans at least 60% of the circumference of a person's arm, wherein these bands are connected to each other, and wherein there are biometric sensors on the relatively-elastic band.

More specifically, the example shown in FIG. 57 is a wearable device for the arm with one or more close-fitting biometric sensors comprising: (a) an inelastic attachment member which is configured to span at least 60% of the circumference of a person's arm, wherein this inelastic attachment member has a first elasticity level; (b) an elastic attachment member which is configured to span at least 60% of the circumference of a person's arm, wherein this elastic attachment member has a second elasticity level, wherein the second elasticity level is greater than the first elasticity level, and wherein the elastic attachment member is connected to the inelastic attachment member; and (c) one or more biometric sensors which are configured to collect data concerning arm tissue, wherein these biometric sensors are part of (or attached to) the elastic attachment member.

In an example, an attachment member can be a band, ring, strap, bracelet, bangle, armlet, sleeve, or cuff. In an example, a band or other attachment member can be attached to a person's arm by connecting two ends of the attachment member with a clasp, clip, buckle, hook, pin, plug, or hook-and-eye mechanism. In an example, a band or other attachment member can be attached to a person's arm by stretching and sliding it over the person's hand onto the arm. In an example, a band or other attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 57 include: inelastic band 5701; elastic band 5702; display screen 5703; and biometric sensors including 5704.

FIG. 58 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example shown in FIG. 58 can be described as an arm-wearable device with two or more modular and connectable bands, wherein each band spans at least 60% of the circumference of a person's arm, and wherein one or more of these bands house biometric sensors.

More specifically, the example shown in FIG. 58 is a wearable device for the arm with one or more close-fitting biometric sensors comprising: (a) a first modular band which is configured to span at least 60% of the circumference of a person's arm; (b) a second modular band which is configured to span at least 60% of the circumference of a person's arm, wherein the first modular band and the second modular band have a first configuration in which they are not connected to each other and are not worn by a person, wherein the first band and the second band have a second configuration wherein they are connected to each other and worn on a person's arm, and wherein the first band and the second band can be changed from the first configuration to the second configuration by the person who wears them, and wherein the first band and the second band can be changed back from the second configuration to the first configuration by the person who wears them; and (c) one or more biometric sensors which are configured to collect data concerning arm tissue, wherein these biometric sensors are part of (or attached to) one or both of the modular bands.

In an example, an attachment member can be a band, ring, strap, bracelet, bangle, armlet, sleeve, or cuff. In an example, a band or other attachment member can be attached to a person's arm by connecting two ends of the attachment member with a clasp, clip, buckle, hook, pin, plug, or hook-and-eye mechanism. In an example, a band or other attachment member can be attached to a person's arm by stretching and sliding it over the person's hand onto the arm. In an example, a band or other attachment member can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 58 include: first modular band 5801; second modular band 5802; temporary connectors 5803 and 5804; and display screens 5805 and 5806.

FIG. 59 shows another example of a wearable device for the arm with one or more close-fitting biometric sensors. This figure shows the device from a side perspective, as it would appear encircling a lateral cross-section of a person's arm.

The example in FIG. 59 can be described as an arm-wearable device with a partial-circumferential inner elastic band and biometric sensors. Such a device can have an outer inelastic band with a first elasticity level which spans a first percentage of the arm circumference and an inner elastic band with a second elasticity level which spans a second percentage of the arm circumference—wherein the second percentage is less than the first percentage and the second elasticity level is greater than the first elasticity level. In the example shown in FIG. 59, an outer inelastic band (and display screen) spans the entire arm circumference and a semi-circular inner elastic band (interior relative to the outer inelastic band) spans only half of the arm circumference. This design can provide an overall semi-rigid structure (for housing a display screen), but can also keep biometric sensors close against the surface of the arm for consistent collection of biometric data.

More specifically, the example shown in FIG. 59 is a wearable device for the arm with one or more close-fitting biometric sensors comprising: (a) an outer inelastic band which is configured to span a first percentage of a person's arm and which has a first elasticity level; (b) an inner elastic band which is configured to span a second percentage of a person's arm and which has a second elasticity level, wherein this inner elastic band is configured to be closer to the surface of the arm than the outer inelastic band, wherein the second percentage is less than the first percentage, and wherein the second elasticity level is greater than the first elasticity level; and (c) one or more biometric sensors which are configured to collect data concerning arm tissue, wherein these biometric sensors are part of (or attached to) the inner elastic band.

Alternatively, the example shown in FIG. 59 can be specified as a wearable device for the arm with one or more close-fitting biometric sensors comprising: (a) an outer inelastic band with a first arcuate length and a first elasticity level; (b) an inner elastic band with a second arcuate length and a second elasticity level, wherein this inner elastic band is located on the concave side of the outer elastic band, wherein the second percentage is less than the first percentage, and wherein the second elasticity level is greater than the first elasticity level; and (c) one or more biometric sensors which are configured to collect data concerning arm tissue, wherein these biometric sensors are part of (or attached to) the inner elastic band.

In an example, the word “ring”, “strap”, “bracelet”, “bangle”, “armlet”, “sleeve”, or “cuff” can be substituted for the word “band” in the above specifications. In an example, an outer inelastic band can span Y % of the circumference of a person's arm and an inner elastic band can span X % of the circumference of a person's arm, wherein Y % is at least 20 percentage points greater than X %. In an example, Y % can be 75% and X % can be 50%. In an example, the ends of the inner elastic band can be attached to the outer inelastic band. In an example, an inner elastic band can be configured to span the anterior (upper) surface of a person's arm. In an example, an inner elastic band can be configured to span the posterior (lower) surface of a person's arm.

In an example, an outer inelastic band can be attached to a person's arm by connecting two ends of an outer inelastic band with a clasp, clip, buckle, hook, pin, plug, or hook-and-eye mechanism. In an example, an outer inelastic band can be attached to a person's arm by stretching and sliding it over the person's hand onto the arm. In an example, an outer inelastic band can be attached to a person's arm by applying force to pull two ends apart to slip the member over the arm, wherein the two ends retract back towards each other when the force is removed.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 59 include: four segments (5901, 5902, 5904, and 5905) of an outer inelastic band; inner elastic band 5907; biometric sensors (5908, 5909, and 5910); outer elastic band clasp 5903; and display screen 5906.

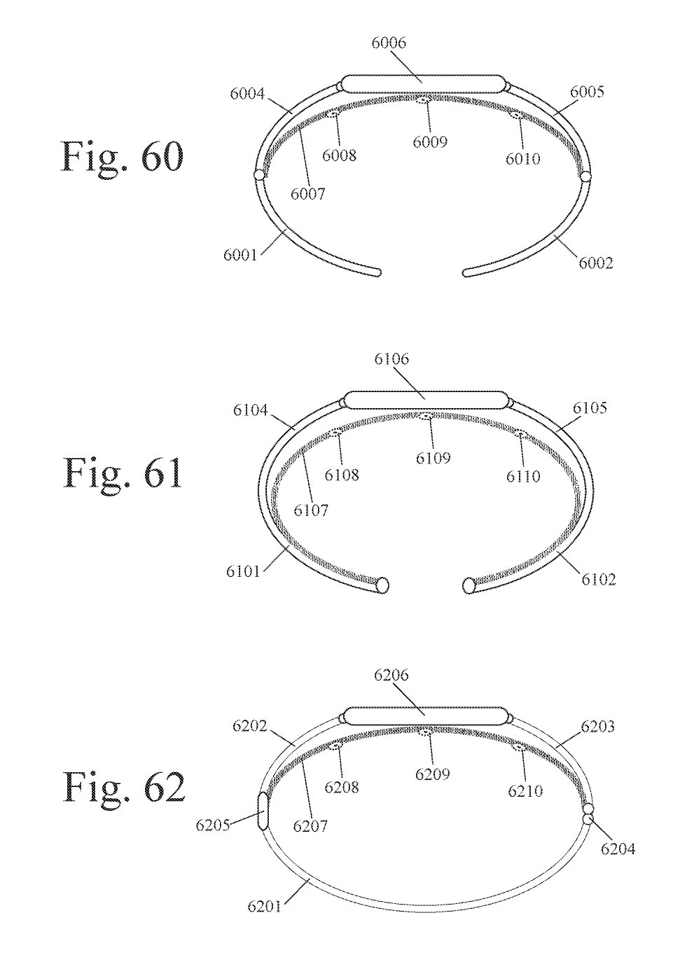

FIG. 60 shows another example of a wearable device for the arm with one or more close-fitting biometric sensors. This figure shows the device from a side perspective, as it would appear encircling a lateral cross-section of a person's wrist (or other portion of their arm). The example in FIG. 60 is like the one shown in FIG. 59, except that in FIG. 60 the outer inelastic band is sufficiently resilient that its ends hold onto the person's arm without the need for a clasp. The outer inelastic band can be attached to a person's arm by applying force to pull two ends apart to slip the member over the arm, wherein the two ends retract back towards each other when the force is removed.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 60 include: four segments (6001, 6002, 6004, and 6005) of an outer inelastic band; inner elastic band 6007; biometric sensors (6008, 6009, and 6010); and display screen 6006.

FIG. 61 shows another example of a wearable device for the arm with one or more close-fitting biometric sensors. This figure shows the device from a side perspective, as it would appear encircling a lateral cross-section of a person's arm. The example in FIG. 61 can be described as an arm-wearable device with an outer arcuate inelastic band, an inner arcuate elastic band, and biometric sensors which are part of the inner band. This design can provide an overall semi-rigid structure (e.g. to hold a rigid display screen in place) and also keep biometric sensors close against the surface of the arm for consistent collection of biometric data.

The example shown in FIG. 61 can be specified as a wearable device for the arm with one or more close-fitting biometric sensors comprising: (a) an outer arcuate inelastic band which is configured to span at least 60% of the circumference of a person's arm and which has a first elasticity level; (b) an inner arcuate elastic band which is located on (and attached to) the concave side of the outer arcuate band and which has a second elasticity level, wherein the second elasticity level is greater than the first elasticity level; and (c) one or more biometric sensors which are configured to collect data concerning arm tissue, wherein these biometric sensors are part of (or attached to) the inner arcuate elastic band. In various examples, a ring, strap, bracelet, bangle, armlet, sleeve, or cuff can be substituted for a band.

Alternatively, the example shown in FIG. 61 can be specified as a wearable device for the arm with one or more close-fitting biometric sensors comprising: (a) an outer arcuate inelastic band, wherein this outer arcuate inelastic band is configured to span at least 60% of the circumference of a person's arm, wherein this outer arcuate inelastic band is configured to be a first average distance from the surface of the person's arm, and wherein this outer arcuate inelastic band has a first elasticity level; (b) an inner arcuate elastic band, wherein this inner arcuate elastic band is attached to the outer arcuate inelastic band, wherein this inner arcuate elastic band is configured to be an second average distance from the surface of the person's arm, wherein this inner arcuate elastic band has a second elasticity level, wherein the second average distance is less than the first average distance, and wherein the second elasticity level is greater than the first elasticity level; and (c) one or more biometric sensors which are configured to collect data concerning arm tissue, wherein these biometric sensors are part of (or attached to) the inner arcuate elastic band. In various examples, a ring, strap, bracelet, bangle, armlet, sleeve, or cuff can be substituted for a band.

In an example, an outer arcuate inelastic band can be attached to a person's arm by connecting two ends of the outer inelastic band with a clasp, clip, buckle, hook, pin, plug, or hook-and-eye mechanism. In an example, an outer arcuate inelastic band can be attached to a person's arm by stretching and sliding it over the person's hand onto the arm. In an example, an outer arcuate inelastic band can be attached to a person's arm by applying force to pull two ends apart to slip the member over the arm, wherein the two ends retract back towards each other when the force is removed. In an example, an inner arcuate elastic band can be made from a stretchable fabric. In an example, an inner arcuate elastic band can be attached to an outer arcuate inelastic band at the ends of the arcuate inelastic band. In an example, an inner arcuate elastic band can be attached to an outer arcuate inelastic band near mid-points of segments of the outer arcuate inelastic band.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 61 include: four segments (6101, 6102, 6104, and 6105) of an outer inelastic band; inner elastic band 6107; biometric sensors (6108, 6109, and 6110); and display screen 6106.

FIG. 62 shows another example of a wearable device for the arm with one or more close-fitting biometric sensors. This figure shows the device from a side perspective, as it would appear encircling a lateral cross-section of a person's wrist (or other portion of the person's arm). The example in FIG. 62 can be described as an arm-wearable device with an outer rigid “clam shell” structure to hold a display screen in place and an inner arcuate elastic band to keep biometric sensors close against the surface of the arm.

The example shown in FIG. 62 can be specified as a wearable device for the arm with one or more close-fitting biometric sensors comprising: (a) a clam shell structure which is configured to span the circumference of a person's arm, wherein this clam shell structure further comprises: an upper half-circumferential portion, a lower half-circumferential portion, a joint (and/or hinge) between these portions on a first side of these portions, and a connector which reversibly connects these portions on a second side of these portions; (b) an arcuate elastic band which is located within the concavity of the clam shell structure and is attached to the clam shell structure; and (c) one or more biometric sensors which are configured to collect data concerning arm tissue, wherein these biometric sensors are part of (or attached to) the arcuate elastic band.

In an example, an upper half-circumferential portion of a clam shell structure can span the anterior (upper) surface of a person's arm and a lower half-circumferential portion of a clam shell structure can span the posterior (lower) surface of the person's arm. In an example, there can be a display screen on the outer surface of one or both portions of a clam shell structure. In an example, a connector which reversibly connects the upper and lower portions of a clam shell structure can be selected from the group consisting of: clasp, clip, buckle, hook, pin, plug, and hook-and-eye mechanism. In an example, an inner arcuate elastic band can be made from a stretchable fabric. In an example, an inner arcuate elastic band can be attached to an upper half-circumferential portion of a clam shell structure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 62 include: two segments 6202 and 6203 of an upper half-circumferential portion of a clam shell structure; a lower half-circumferential portion 6201 of the clam shell structure; a joint (or hinge) 6204 between the upper and lower portions of the clam shell structure; a reversible connector 6205 between the upper and lower portions of the clam shell structure; an inner elastic band 6207; biometric sensors 6208, 6209, and 6210; and display screen 6206.

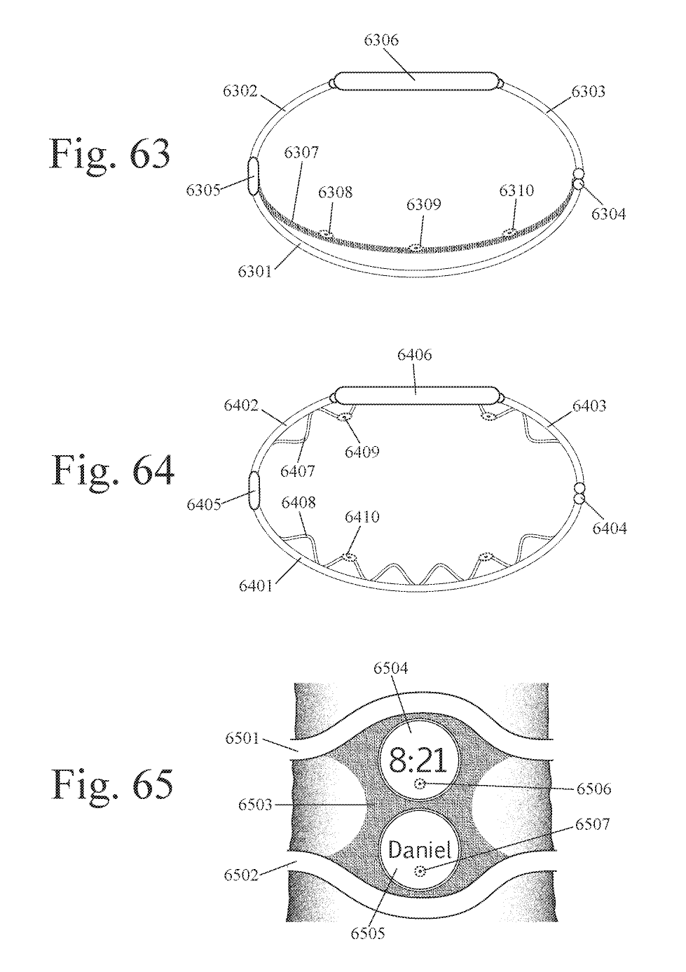

FIG. 63 shows another example of a wearable device for the arm with one or more close-fitting biometric sensors. This figure shows the device from a side perspective, as it would appear encircling a lateral cross-section of a person's wrist (or other portion of the person's arm). The example in FIG. 63 is like the one shown in FIG. 62, except that in FIG. 63 an inner arcuate elastic band spans the posterior (lower) surface of a person's arm. Specific components in the example shown in FIG. 63 include: two segments 6302 and 6303 of an upper half-circumferential portion of a clam shell structure; a lower half-circumferential portion 6301 of the clam shell structure; a joint (or hinge) 6304 between the upper and lower portions of the clam shell structure; a reversible connector 6305 between the upper and lower portions of the clam shell structure; an inner elastic band 6307; biometric sensors 6308, 6309, and 6310; and display screen 6306.

FIG. 64 shows another example of a wearable device for the arm with one or more close-fitting biometric sensors. This figure shows the device from a side perspective, as it would appear encircling a lateral cross-section of a person's wrist (or other portion of the person's arm). The example in FIG. 64 can be described as an arm-wearable device with an outer rigid “clam shell” structure and inward-facing flexible undulations to keep biometric sensors close against the surface of the arm.

The example shown in FIG. 64 can be specified as a wearable device for the arm with one or more close-fitting biometric sensors comprising: (a) a clam shell structure which is configured to span the circumference of a person's arm, wherein this clam shell structure further comprises: an upper half-circumferential portion, a lower half-circumferential portion, a joint (and/or hinge) between these portions on a first side of these portions, and a connector which reversibly connects these portions on a second side of these portions; (b) an inward-facing undulating member which is part of (or attached to) the clam shell structure; and (c) one or more biometric sensors which are configured to collect data concerning arm tissue, wherein these biometric sensors are part of (or attached to) the undulating member.

In an example, an upper half-circumferential portion of a clam shell structure can span the anterior (upper) surface of a person's arm and a lower half-circumferential portion of a clam shell structure can span the posterior (lower) surface of the person's arm. In an example, there can be a display screen on the outer surface of one or both portions of a clam shell structure. In an example, a connector which reversibly connects the upper and lower portions of a clam shell structure can be selected from the group consisting of: clasp, clip, buckle, hook, pin, plug, and hook-and-eye mechanism. In an example, an inward-facing undulating member can have a sinusoidal shape. In an example, an inward-facing undulating member can be flexible and/or compressible. In an example, an inward-facing undulating member can be elastic and filled with a liquid, gel, or gas.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 64 include: two segments 6402 and 6403 of an upper half-circumferential portion of a clam shell structure; a lower half-circumferential portion 6401 of the clam shell structure; a joint (or hinge) 6404 between the upper and lower portions of the clam shell structure; a reversible connector 6405 between the upper and lower portions of the clam shell structure; inward-facing undulating members including 6407 and 6408; biometric sensors including 6409 and 6410; and display screen 6406.

FIG. 65 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example in FIG. 65 can be described as an arm-wearable device with two display screens suspended by an elastic material between two arcuate bands.

The example shown in FIG. 65 can be specified as a wearable device for the arm with one or more close-fitting biometric sensors comprising: (a) a distal arcuate band which is configured to span at least 60% of the circumference of a person's arm; (b) a proximal arcuate band which is configured to span at least 60% of the circumference of a person's arm, wherein distal is defined as further from a person's shoulder and proximal is defined as closer to the person's shoulder; (c) an elastic member that is between the distal arcuate band and the proximal arcuate band which connects the distal actuate band to the proximal arcuate band; and (d) two arcuate display screens between the distal arcuate band and the proximal arcuate band, wherein these display screens are attached to the elastic member; and (e) one or more biometric sensors which are configured to collect data concerning arm tissue. In various examples, a ring, strap, bracelet, or bangle can be substituted for a band.

In an example, an arcuate band can undulate laterally as it spans the circumference a person's arm. In an example, distal and proximal arcuate bands can curve away from each other as they span a central portion of the anterior (upper) surface of a person's arm and can curve back toward each other as they span a side surface of the person's arm. In an example, an arcuate band can be attached to a person's arm by connecting two ends of the arcuate band with a clasp, clip, buckle, hook, pin, plug, or hook-and-eye mechanism. In an example, an arcuate band can be attached to a person's arm by stretching and sliding it over the person's hand onto the arm. In an example, an arcuate band can be attached to a person's arm by applying force to pull two ends apart to slip the member over the arm, wherein the two ends retract back towards each other when the force is removed.

In an example, an elastic member can be made from elastic fabric. In an example, an elastic member can be an elastic mesh. In an example, an elastic member can have four arcuate sides: two convex sides and two concave sides. In an example, one concave side can connect to the distal arcuate band and the other concave side can connect to the proximal band. In an example, two convex sides can be between the two bands. In an example, an elastic member can completely surround the perimeters of two display screens. In an example, an elastic member can flexibly-suspend two display screens between two arcuate bands. In an example, a display screen can be circular. In an example, the centers of two display screens can be connected to form a virtual line which is parallel to the longitudinal axis of an arm. In an example, the centers of two display screens can be connected to form a virtual line which is parallel to a line which is perpendicular to the circumferences of distal and proximal arcuate bands.

In an example, biometric sensors can be part of (or attached to) display screens and/or enclosures which house display screens. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 65 include: distal arcuate band 6501; proximal arcuate band 6502; elastic member 6503 between the two arcuate bands; display screens 6504 and 6505 suspended by the elastic member; and biometric sensors 6506 and 6507.

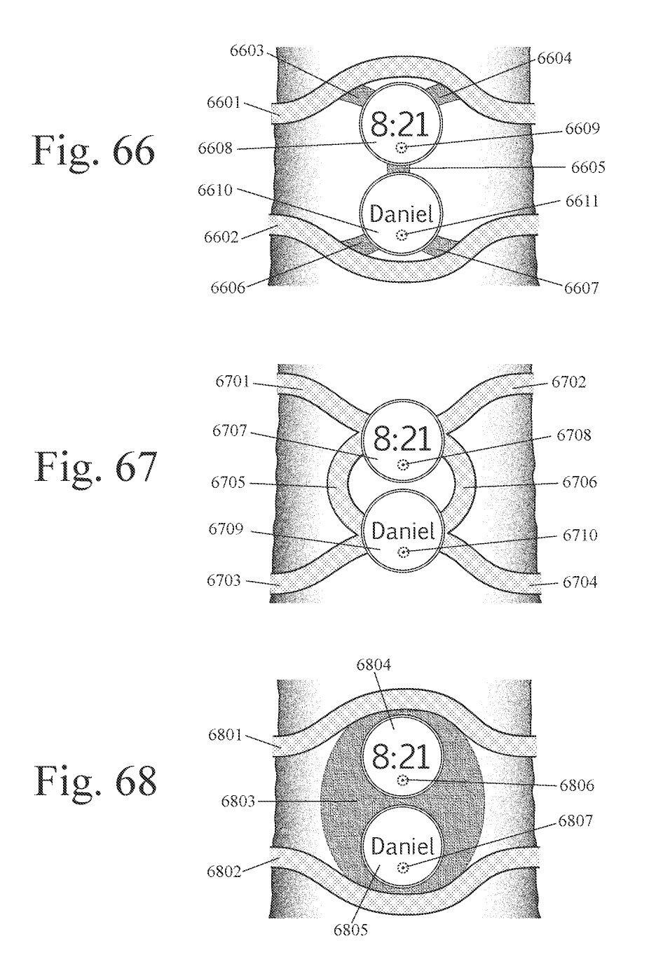

FIG. 66 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example in FIG. 66 can be described as an arm-wearable device with two display screens which are suspended by elastic straps between two arcuate bands.

The example shown in FIG. 66 can be specified as a wearable device for the arm with one or more close-fitting biometric sensors comprising: (a) a distal arcuate band which is configured to span at least 60% of the circumference of a person's arm; (b) a proximal arcuate band which is configured to span at least 60% of the circumference of a person's arm, wherein distal is further from a person's shoulder and proximal is closer to the person's shoulder; (c) a plurality of elastic straps between the distal arcuate band and the proximal arcuate band which connect the distal actuate band to the proximal arcuate band; and (d) two arcuate display screens between the distal arcuate band and the proximal arcuate band, wherein these display screens are attached to the plurality of elastic straps; and (e) one or more biometric sensors which are configured to collect data concerning arm tissue. In an example, a ring, bracelet, or bangle can be substituted for a band.

In an example, an arcuate band can undulate laterally as it spans the circumference a person's arm. In an example, distal and proximal arcuate bands can curve away from each other as they span a central portion of the anterior (upper) surface of a person's arm and can curve back toward each other as they span a side surface of the person's arm. In an example, an arcuate band can be attached to a person's arm by connecting two ends of the arcuate band with a clasp, clip, buckle, hook, pin, plug, or hook-and-eye mechanism. In an example, an arcuate band can be attached to a person's arm by stretching and sliding it over the person's hand onto the arm. In an example, an arcuate band can be attached to a person's arm by applying force to pull two ends apart to slip the member over the arm, wherein the two ends retract back towards each other when the force is removed.

In an example, an elastic strap can be made from elastic fabric. In an example, an elastic strap can be an elastic mesh. In an example, each display screen can be connected to three elastic straps. In an example, each display screen can be connected to three elastic straps with connection points which are substantially equidistant around the circumference of a display screen. In an example, each arcuate band can be connected to two elastic straps. In an example, two display screens can be connected by one elastic strap. In an example, there can be five elastic straps in total. In an example, a display screen can be circular. In an example, the centers of two display screens can be connected to form a virtual line which is parallel to the longitudinal axis of an arm. In an example, the centers of two display screens can be connected to form a virtual line which is parallel to a line which is perpendicular to the circumferences of distal and proximal arcuate bands.

In an example, biometric sensors can be part of (or attached to) display screens and/or enclosures which house display screens. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 66 include: distal arcuate band 6601; proximal arcuate band 6602; a plurality of elastic straps including 6603, 6604, 6605, 6606, and 6607; display screens 6608 and 6610 suspended by the elastic straps; and biometric sensors 6609 and 6611.

FIG. 67 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example in FIG. 67 can be described as an arm-wearable device with two display screens whose centers are at 12 o'clock and 6 o'clock positions around a circular band on the anterior (upper) surface of an arm.

The example shown in FIG. 67 can be specified as a wearable device for the arm with one or more close-fitting biometric sensors comprising: (a) a distal arcuate band which is configured to span at least 60% of the circumference of a person's arm; (b) a proximal arcuate band which is configured to span at least 60% of the circumference of a person's arm, wherein distal is further from a person's shoulder and proximal is closer to the person's shoulder; (c) a distal display screen on the distal arcuate band; (d) a proximal display screen on the proximal arcuate band; (e) a right circle-segment band which connects the right side of the distal display screen to the right side of the proximal display screen; (f) a left circle-segment band which connects the left side of the distal display screen to the left side of the proximal display screen; and (g) one or more biometric sensors which are configured to collect data concerning arm tissue.

In an example, an arcuate band can be attached to a person's arm by connecting two ends of the arcuate band with a clasp, clip, buckle, hook, pin, plug, or hook-and-eye mechanism. In an example, an arcuate band can be attached to a person's arm by stretching and sliding it over the person's hand onto the arm. In an example, an arcuate band can be attached to a person's arm by applying force to pull two ends apart to slip the member over the arm, wherein the two ends retract back towards each other when the force is removed. In an example, a display screen can be circular. In an example, the centers of two display screens can be connected to form a virtual line which is parallel to the longitudinal axis of an arm. In an example, the centers of two display screens can be connected to form a virtual line which is parallel to a line which is perpendicular to the circumferences of distal and proximal arcuate bands.

In an example, biometric sensors can be part of (or attached to) display screens and/or enclosures which house display screens. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 67 include: portions 6701 and 6702 of a distal arcuate band; portions 6703 and 6704 of a proximal arcuate band; display screens 6707 and 6709; right circle-segment band 6706; left circle-segment band 6705; and biometric sensors 6708 and 6710.

FIG. 68 shows another example of a wearable device for the arm with one or more close-fitting biometric sensors. This figure shows the device from a top-down perspective, as it would appear spanning the anterior (upper) surface of a person's wrist (or other portion of the person's arm) in a circumferential manner. The example in FIG. 68 can be described as an arm-wearable device with two display screens suspended by an oval (or elliptical or circular) elastic member between two arcuate bands.

The example shown in FIG. 68 can be specified as a wearable device for the arm with one or more close-fitting biometric sensors comprising: (a) a distal arcuate band which is configured to span at least 60% of the circumference of a person's arm; (s) a proximal arcuate band which is configured to span at least 60% of the circumference of a person's arm, wherein distal is defined as further from a person's shoulder and proximal is defined as closer to the person's shoulder; (t) an oval (or elliptical or circular) elastic member that is between the distal arcuate band and the proximal arcuate band which connects the distal actuate band to the proximal arcuate band; and (r) two arcuate display screens between the distal arcuate band and the proximal arcuate band, wherein these display screens are attached to the oval (or elliptical or circular) elastic member; and (o) one or more biometric sensors which are configured to collect data concerning arm tissue. In various examples, a ring, strap, bracelet, or bangle can be substituted for a band.

In an example, an arcuate band can undulate laterally as it spans the circumference a person's arm. In an example, distal and proximal arcuate bands can curve away from each other as they span a central portion of the anterior (upper) surface of a person's arm and can curve back toward each other as they span a side surface of the person's arm. In an example, an arcuate band can be attached to a person's arm by connecting two ends of the arcuate band with a clasp, clip, buckle, hook, pin, plug, or hook-and-eye mechanism. In an example, an arcuate band can be attached to a person's arm by stretching and sliding it over the person's hand onto the arm. In an example, an arcuate band can be attached to a person's arm by applying force to pull two ends apart to slip the member over the arm, wherein the two ends retract back towards each other when the force is removed.

In an example, an oval (or elliptical or circular) elastic member can be made from elastic fabric. In an example, an oval (or elliptical or circular) elastic member can be an elastic mesh. In an example, an oval (or elliptical or circular) elastic member can completely surround the perimeters of two display screens. In an example, an oval (or elliptical or circular) elastic member can flexibly-suspend two display screens between two arcuate bands. In an example, a display screen can be circular. In an example, the centers of two display screens can be connected to form a virtual line which is parallel to the longitudinal axis of an arm. In an example, the centers of two display screens can be connected to form a virtual line which is parallel to a line which is perpendicular to the circumferences of distal and proximal arcuate bands.

In an example, biometric sensors can be part of (or attached to) display screens and/or enclosures which house display screens. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 68 include: distal arcuate band 6801; proximal arcuate band 6802; oval (or elliptical or circular) elastic member 6803 between the two arcuate bands; display screens 6804 and 6805 suspended by the oval (or elliptical or circular) elastic member; and biometric sensors 6806 and 6807.

In an example, a wearable spectroscopic sensor to measure food consumption based on the interaction between light and the human body can comprise a wearable food-consumption monitor that is configured to be worn on a person's wrist, arm, hand or finger. In an example, a wearable spectroscopic sensor to measure food consumption based on the interaction between light and the human body can monitor light energy that is reflected from a person's body tissue, absorbed by the person's body tissue, or has passed through the person's body tissue. In an example, a wearable spectroscopic sensor to measure food consumption based on the interaction between light and the human body can identify consumption of a selected type of food, ingredient, or nutrient using spectral analysis. In an example, a spectroscopic sensor can be a white light spectroscopic sensor, an infrared spectroscopic sensor, a near-infrared spectroscopic sensor, an ultraviolet spectroscopic sensor, an ion mobility spectroscopic sensor, a mass spectrometry sensor, a backscattering spectrometry sensor, or a spectrophotometer.

In an example, a wearable device to measure a person's food consumption based on the interaction between light energy and the person's body can comprise: (a) at least one wearable spectroscopic sensor that collects data concerning the spectrum of light energy reflected from a person's body tissue, absorbed by the person's body tissue, and/or having passed through the person's body tissue, wherein this data is used to measure the person's consumption of one or more selected types of food, ingredients, and/or nutrients; (b) a data processing unit; and (c) a power source.

In an example, a device can further comprise an attachment member selected from the group consisting of: finger ring, smart watch, wrist band, wrist bracelet, armlet, cuff, and sleeve. In an example, a device can be configured to be worn on a person's finger, hand, wrist, and/or arm. In an example, a spectroscopic sensor can be selected from the group consisting of: white light spectroscopic sensor, infrared light spectroscopic sensor, near-infrared light spectroscopic sensor, and ultraviolet light spectroscopic sensor. In an example, a spectroscopic sensor can be selected from the group consisting of spectrometer, spectrophotometer, ion mobility spectroscopic sensor, and backscattering spectrometry sensor.

In an example, this device can further comprise a first spectroscopic sensor at a first location on the device and a second spectroscopic sensor at a second location on the device, wherein the distance along a circumference of the device from the first location to the second location is at least a quarter inch. In an example, a spectroscopic sensor can be moved along the circumference of the device. In an example, moving the spectroscopic sensor along the circumference of the device changes the location of the spectroscopic sensor relative to the person's body.

In an example, a device can further comprise a first spectroscopic sensor which is configured to project a beam of light onto the surface of a person's body at a first angle and a second spectroscopic sensor which is configured to project a beam of light onto the surface of the person's body at a second angle, wherein the first angle differs from the second angle by at least 10 degrees. In an example, a spectroscopic sensor can be rotated relative to the rest of the device. In an example, rotating the spectroscopic sensor changes the angle at which the spectroscopic sensor projects a beam of light onto the surface of the person's body.

In an example, a device can further comprise an elastic member filled with a flowable substance (such as a gas or liquid) and this elastic member pushes a spectroscopic sensor toward the surface of the person's body. In an example, a device can further comprise an elastic strap (or band) spanning less than 60% of the circumference of the device and this elastic strap (or band) pushes or pulls a spectroscopic sensor toward the surface of the person's body. In an example, a device can further comprise a spring which pushes or pulls a spectroscopic sensor toward the surface of the person's body.

In an example, this device can further comprise: (a) an attachment member which is configured to span at least 60% of the circumference of a person's wrist and/or arm, wherein this attachment member further comprises one or more elastic portions which are configured to span the anterior surface of the person's wrist and/or arm and one or more inelastic portions which are configured to span the posterior surface of the person's wrist and/or arm; and (b) an enclosure which is connected to the elastic portions of the attachment member, wherein a spectroscopic sensor is part of the enclosure.

In an example, this device can further comprise: (a) an attachment member which is configured to span at least 60% of the circumference of a person's wrist and/or arm, wherein this attachment member further comprises one or more anterior inelastic portions which are configured to span the anterior surface of the person's wrist and/or arm, one or more posterior inelastic portions which are configured to span the posterior surface of the person's wrist and/or arm, and one or more elastic portions which connect the anterior and posterior inelastic portions; and (b) an enclosure which is configured to be worn on the anterior portion of the wrist and/or arm, wherein a spectroscopic sensor is part of the enclosure.

In an example, this device can further comprise: (a) an attachment member which is configured to span at least 60% of the circumference of a person's wrist and/or arm, wherein this attachment member further comprises a first elastic portion with a first elasticity level, a second elastic portion with a second elasticity level, and an inelastic portion with a third elasticity level, wherein the third elasticity level is less than each of the first and second elasticity levels; and (b) an enclosure which is connected between the first and second elastic portions, wherein a spectroscopic sensor is part of the enclosure.

In an example, this device can further comprise: (a) an outer inelastic band which is configured to span a first percentage of a person's wrist and/or arm and which has a first elasticity level; (b) an inner elastic band which is configured to span a second percentage of the person's wrist and/or arm and which has a second elasticity level, wherein the inner elastic band is configured to be closer to the surface of the wrist and/or arm than the outer inelastic band, wherein the second percentage is less than the first percentage, and wherein the second elasticity level is greater than the first elasticity level, and wherein a spectroscopic sensor is part of the inner elastic band.

In an example, this device can further comprise: (a) an outer arcuate inelastic band which is configured to span at least 60% of the circumference of a person's wrist and/or arm and which has a first elasticity level; (b) an inner arcuate elastic band which is located on the concave side of the outer arcuate band and which has a second elasticity level, wherein the second elasticity level is greater than the first elasticity level, and wherein a spectroscopic sensor is part of the inner arcuate elastic band.

In an example, this device can further comprise a clam shell structure which is configured to span the circumference of a person's wrist and/or arm, wherein this clam shell structure further comprises: (a) an upper half-circumferential portion, (b) a lower half-circumferential portion, (c) a joint between these portions on a first side of these portions, and (d) a connector which reversibly connects these portions on a second side of these portions; and (e) an inward-facing undulating member which is part of or attached to the clam shell structure, wherein a spectroscopic sensor is part of or attached to the undulating member.

In an example, this device can further comprise: (a) circumferentially-undulating attachment member which is configured to span at least a portion of the circumference of a person's wrist and/or arm; and (b) a plurality of spectroscopic sensors which collect data concerning wrist and/or arm tissue, wherein each sensor is located at the proximal portion of an undulation, and wherein the proximal portion of an undulation is the portion of an undulating wave which is closest to the circumferential center of the device.

In an example, this device can further comprise: (a) an attachment member which is configured to span at least 60% of the circumference of a person's wrist and/or arm; (b) an enclosure, wherein a spectroscopic sensor is part of the enclosure; and (c) a plurality of elastic, stretchable, and/or springy suspension members, wherein these suspension members flexibly connect the enclosure to the attachment member, wherein each of these suspension members is connected at one end to a location on the circumference of the enclosure and connected at its other end to the attachment member, and wherein the longitudinal axis of each of the suspension members is substantially parallel with a virtual radial spoke outward from the center of the enclosure.

In an example, a glucose monitoring and managing system can comprise: a wearable glucose-monitoring microwave sensor that collects data which is used to measure a person's intra-body glucose levels; and a wearable or implanted pump which delivers a glucose-level-modifying substance into the person's body based on data collected by the wearable glucose-monitoring microwave sensor. In an example, a closed-loop glucose monitoring and managing system can comprise: a wearable glucose-monitoring microwave sensor which automatically collects data which is used to measure a person's intra-body glucose levels; and a wearable or implanted pump which automatically delivers a glucose-level-modifying substance into the person's body based on data collected by the wearable glucose-monitoring microwave sensor. In an example, a closed-loop glucose monitoring and managing system can comprise: a wearable glucose-monitoring microwave sensor which automatically collects data which is used to measure a person's intra-body glucose levels; and a wearable or implanted pump which automatically delivers a glucose-level-modifying substance into the person's body to maintain intra-body glucose levels within a selected range, wherein operation of the pump is based on data collected by the wearable glucose-monitoring microwave sensor.

In an example, a wearable glucose-monitoring microwave sensor can comprise a microwave energy emitter and a microwave energy receiver, wherein transmission of microwave energy from the microwave energy emitter to the microwave energy receiver is changed by changes in the glucose levels of nearby body tissue and/or fluid. In an example, a wearable glucose-monitoring microwave sensor can collect data concerning microwave energy which is transmitted through body tissue and/or fluid, wherein this transmitted microwave energy is changed by changes in the level of glucose in the body tissue and/or fluid. In an example, a wearable glucose-monitoring microwave sensor can collect data concerning microwave energy which is reflected from body tissue and/or fluid, wherein this reflected microwave energy is changed by changes in the level of glucose in the body tissue and/or fluid. In an example, a wearable glucose-monitoring microwave sensor can collect data concerning the electromagnetic interaction between transmitted microwave energy and nearby body tissue and/or fluid, wherein this interaction is changed by changes in the level of glucose in the body tissue and/or fluid.

In an example, a glucose monitoring and managing system can comprise: a wearable glucose-monitoring microwave sensor which collects data which is used to measure a person's intra-body glucose levels, wherein this glucose-monitoring microwave sensor further comprises a microwave energy emitter and a microwave energy receiver; and a wearable or implanted pump which delivers a glucose-level-modifying substance into the person's body based on data collected by the wearable glucose-monitoring microwave sensor. In an example, a closed-loop glucose monitoring and managing system can comprise: a wearable glucose-monitoring microwave sensor which automatically collects data which is used to measure a person's intra-body glucose levels, wherein this glucose-monitoring microwave sensor further comprises a microwave energy emitter and a microwave energy receiver; and a wearable or implanted pump which automatically delivers a glucose-level-modifying substance into the person's body to maintain intra-body glucose levels within a selected range, wherein operation of the pump is based on data collected by the wearable glucose-monitoring microwave sensor.

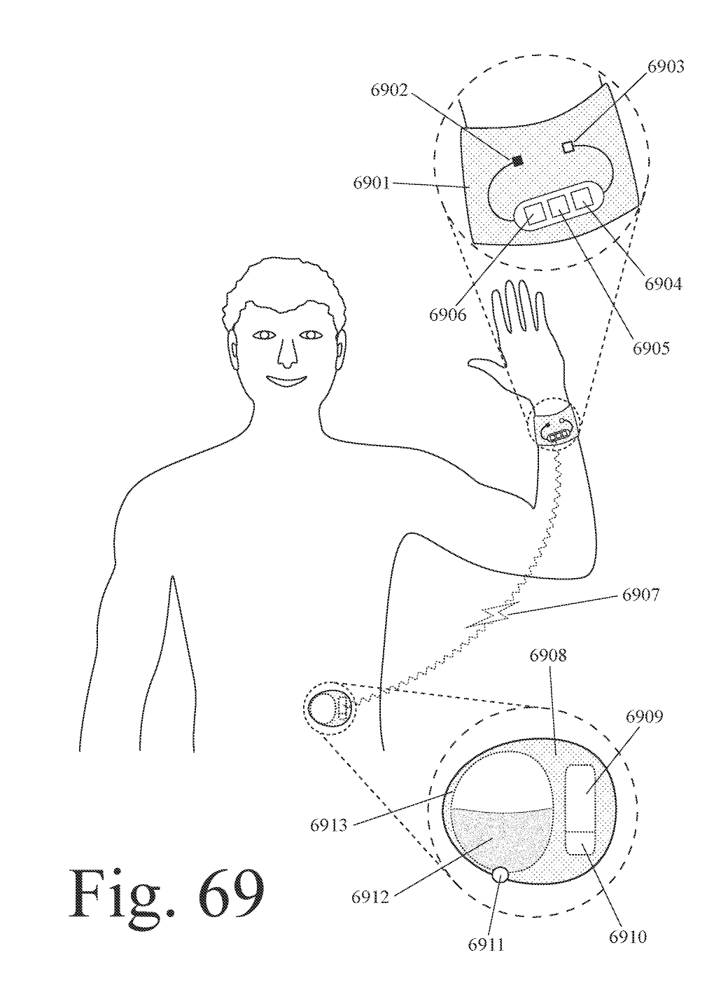

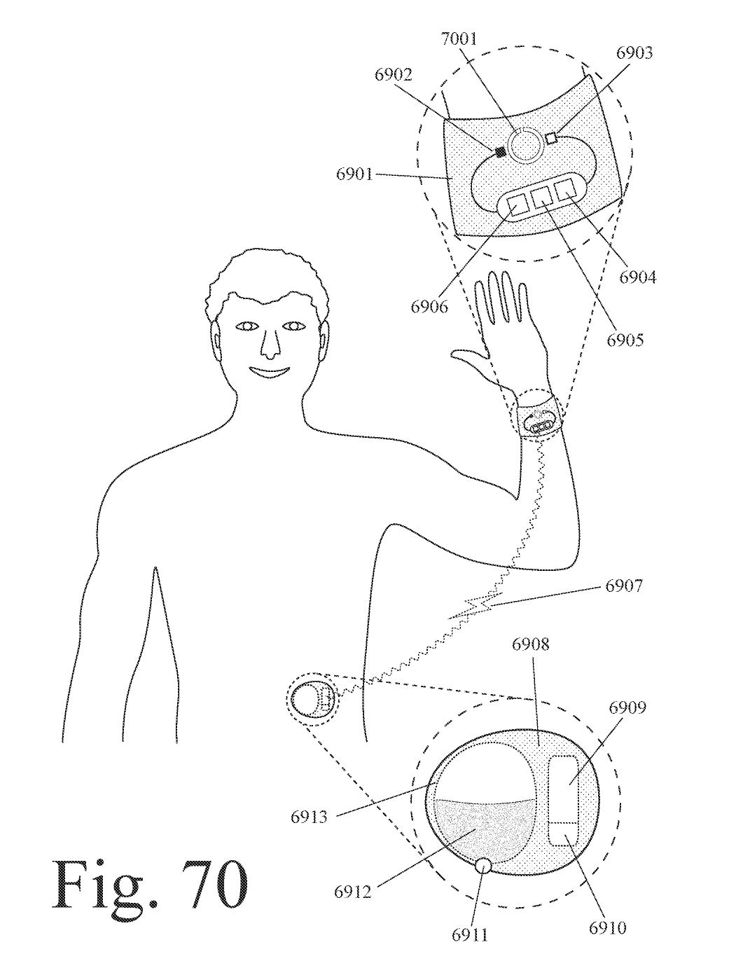

FIG. 69 shows an example of how this invention can be embodied in a glucose monitoring and managing system comprising: a wearable glucose-monitoring microwave sensor which collects data which is used to measure a person's intra-body glucose levels, wherein this glucose-monitoring microwave sensor further comprises a microwave energy emitter and a microwave energy receiver; and a wearable or implanted pump which delivers a glucose-level-modifying substance into the person's body based on data collected by the wearable glucose-monitoring microwave sensor.

The middle portion of FIG. 69 shows a frontal view of a person's head, torso, and hand, wherein the person is wearing a wearable glucose-monitoring microwave sensor on their wrist and a wearable pump on their torso which dispenses a glucose-level-modifying substance. In this example, the wrist-worn component and the wearable pump component are in direct wireless electromagnetic communication with each other. The operation of the wearable pump is controlled based on analysis of data from microwave sensor in the wrist-worn component. In this example, the components shown in FIG. 69 combine to form a system for monitoring and managing intra-body glucose levels.

The upper right portion of FIG. 69 shows a close-up view of the wrist-worn component of this system within a virtual dashed-line circle, wherein this dashed-line circle corresponds to the smaller dashed-line circle around the same wrist-worn component shown in smaller scale on the middle portion of the figure. The lower right portion of FIG. 69 shows a close-up view of the wearable pump component of this system within a virtual-dashed line circle, wherein this dashed-line circle corresponds to the smaller dashed line circle around the same wearable pump component that is shown in smaller scale on the middle portion of the figure.

With respect to specific structures within system components, the wearable glucose-monitoring microwave sensor shown in a close-up view in the upper right portion of FIG. 69 comprises: wrist band 6901; microwave energy emitter 6902; microwave energy receiver 6903; power source 6904; data processor 6905; and data transmitter/receiver 6906. With respect to structures within system components, the pump shown in a close-up view in the lower right portion of FIG. 69 includes: wearable pump housing 6908; data processor and transmitter/receiver 6909; power source 6910; glucose-level-modifying substance 6912; pump 6911 for the glucose-level-modifying substance; and reservoir 6913 for the glucose-level-modifying substance. In this example, data transmitter/receiver 6906 of the wrist-worn component is in direct wireless communication 6907 with data processor and transmitter/receiver 6909 of the wearable pump component. In another example, both of these data transmitters/receivers can be in indirect wireless communication by each having wireless communication with a common separate and/or remote data processor and transmitter/receiver. Relevant example variations which are discussed in other portions of this disclosure can also be applied to the embodiment shown here in FIG. 69.

In another example, a wearable glucose-monitoring microwave sensor can comprise a microwave energy emitter, a microwave energy receiver, and a resonator between the microwave energy emitter and the microwave energy receiver, wherein the resonant frequency of the resonator is changed by changes in the glucose levels of nearby body tissue and/or fluid. In an example, changes in the glucose levels of nearby body tissue and/or fluid change the permittivity of the body tissue and/or fluid which, in turn, changes the resonant frequency of the resonator. In an example, a wearable glucose-monitoring microwave sensor can comprise a microwave energy emitter, a microwave energy receiver, and a resonator, wherein changes in microwave transmission from the microwave energy emitter to the microwave energy receiver are used to measure changes in the glucose levels of nearby body tissue and/or fluid.

In an example, a glucose monitoring and managing system can comprise: a wearable glucose-monitoring microwave sensor which collects data which is used to measure a person's intra-body glucose levels, wherein this glucose-monitoring microwave sensor further comprises a microwave energy emitter, a microwave energy receiver, and a resonator between the microwave energy emitter and the microwave energy receiver; and a wearable or implanted pump which delivers a glucose-level-modifying substance into the person's body based on data collected by the wearable glucose-monitoring microwave sensor. In an example, one or more resonant frequencies of such a resonator can change with changes in the glucose level of nearby body tissue and/or body fluid. One or more resonant frequencies of such a resonator can be changed by a change in the permittivity of nearby body tissue and/or fluid which, in turn, can be changed by changes in the glucose level of nearby body tissue and/or body fluid. The resonant frequency of a resonator is reduced by dielectric loading of body tissue. In an example, there can be an inverse relationship between the glucose levels of body tissue and/or fluid and the resonant frequency of a resonator. In this manner, intra-body glucose levels can be estimated by measuring one or more resonant frequencies of an electromagnetic resonator.

In an example, a closed-loop glucose monitoring and managing system can comprise: a wearable glucose-monitoring microwave sensor which automatically collects data which is used to measure a person's intra-body glucose levels, wherein this glucose-monitoring microwave sensor further comprises a microwave energy emitter, a microwave energy receiver, and a resonator between the microwave energy emitter and the microwave energy receiver; and a wearable or implanted pump which automatically delivers a glucose-level-modifying substance into the person's body to maintain intra-body glucose levels within a selected range, wherein operation of the pump is based on data collected by the wearable glucose-monitoring microwave sensor.

FIG. 70 shows an example of how this invention can be embodied in a glucose monitoring and managing system comprising: a wearable glucose-monitoring microwave sensor that collects data which is used to measure a person's intra-body glucose levels, wherein this glucose-monitoring microwave sensor further comprises a microwave energy emitter, a microwave energy receiver, and a resonator; and a wearable or implanted pump which delivers a glucose-level-modifying substance into the person's body based on data collected by the wearable glucose-monitoring microwave sensor.

The middle portion of FIG. 70 shows a frontal view of a person's head, torso, and hand, wherein the person is wearing a wearable glucose-monitoring microwave sensor on their wrist and a wearable pump on their torso which dispenses a glucose-level-modifying substance. In this example, the wrist-worn component and the wearable pump component are in direct wireless electromagnetic communication with each other. The operation of the wearable pump is controlled based on analysis of data from microwave sensor in the wrist-worn component. In this example, the components shown in FIG. 70 combine to form a system for monitoring and managing intra-body glucose levels.

The upper right portion of FIG. 70 shows a close-up view of the wrist-worn component of this system within a virtual dashed-line circle, wherein this dashed-line circle corresponds to the smaller dashed-line circle around the same wrist-worn component shown in smaller scale on the middle portion of the figure. The lower right portion of FIG. 70 shows a close-up view of the wearable pump component of this system within a virtual-dashed line circle, wherein this dashed-line circle corresponds to the smaller dashed line circle around the same wearable pump component that is shown in smaller scale on the middle portion of the figure.

With respect to specific structures within system components, the wearable glucose-monitoring microwave sensor shown in a close-up view in the upper right portion of FIG. 70 comprises: wrist band 6901; microwave energy emitter 6902; resonator 7001; microwave energy receiver 6903; power source 6904; data processor 6905; and data transmitter/receiver 6906. With respect to structures within system components, the pump shown in a close-up view in the lower right portion of FIG. 70 includes: wearable pump housing 6908; data processor and transmitter/receiver 6909; power source 6910; glucose-level-modifying substance 6912; pump 6911 for the glucose-level-modifying substance; and reservoir 6913 for the glucose-level-modifying substance. In this example, data transmitter/receiver 6906 of the wrist-worn component is in direct wireless communication 6907 with data processor and transmitter/receiver 6909 of the wearable pump component. In another example, both of these data transmitters/receivers can be in indirect wireless communication by each having wireless communication with a common separate and/or remote data processor and transmitter/receiver. Relevant example variations which are discussed in other portions of this disclosure can also be applied to the embodiment shown here in FIG. 70.

In an example, a glucose monitoring and managing system can comprise: a wearable glucose-monitoring microwave sensor which collects data which is used to measure a person's intra-body glucose levels; and a wearable or implanted pump which delivers a glucose-level-modifying substance into the person's body based on data collected by the wearable glucose-monitoring microwave sensor. In an example, a glucose monitoring and managing system can comprise: a wearable glucose-monitoring microwave sensor which measures intra-body glucose level; and a wearable or implanted insulin pump, wherein the pump automatically dispenses insulin when needed based on data from the wearable glucose-monitoring microwave sensor. In an example, a glucose monitoring and managing system can comprise: a wearable glucose-monitoring microwave sensor which collects data concerning electromagnetic energy that is transmitted through and/or reflected from body tissue and/or fluid, wherein this data is analyzed to measure a person's intra-body glucose level; and a wearable or implanted insulin pump, wherein the pump automatically dispenses insulin when needed based on analysis of data from the wearable glucose-monitoring microwave sensor.

In an example, a wearable glucose-monitoring microwave sensor can be a permittivity sensor. The permittivity of body tissue and/or fluid is the ability of body tissue and/or fluid to transmit an electromagnetic field. Permittivity depends on the amount of electrical energy that is stored within the body tissue and/or fluid when the body tissue and/or fluid is exposed to an electromagnetic field. In an example, a wearable glucose-monitoring microwave sensor can measure glucose levels in nearby body fluid and/or tissue by measuring the permittivity of that body fluid and/or tissue. In an example, a wearable glucose-monitoring microwave sensor can measure changes glucose levels in nearby body fluid and/or tissue by measuring changes in the permittivity of that body fluid and/or tissue. In an example, changes in the glucose levels of body tissue and/or fluid can change the permittivity of that body tissue and/or fluid, wherein these changes in turn can be measured by a wearable glucose-monitoring microwave sensor. In an example, changes in glucose levels in body tissue and/or fluid change the permittivity of cell membranes. In an example, the permittivity and glucose levels of blood can be measured by a wearable glucose-monitoring microwave sensor.

In an example, the permittivity of body tissue and/or fluid can be different at different microwave energy frequencies. In an example, a wearable glucose-monitoring microwave sensor can emit microwave energy at varying frequencies to collect data on the electromagnetic interaction between that energy and body tissue and/or fluid at different frequencies. In an example, a wearable glucose-monitoring microwave sensor can sweep through a selected range of microwave frequencies. In an example, a wearable glucose-monitoring microwave sensor can be an electromagnetic spectroscopy sensor which collects data on the permittivity of body tissue and/or fluid across a selected (sub)spectrum of microwave frequencies. In an example, collecting data on the permittivity of body tissue and/or fluid across a range of microwave frequencies can provide more accurate estimation of intra-body glucose levels than collecting data on permittivity at a single microwave frequency.

In an example, a wearable glucose-monitoring microwave sensor can be a dielectric constant sensor. A dielectric constant is the real part of permittivity. In an example, a wearable glucose-monitoring microwave sensor can measure glucose levels in nearby body fluid and/or tissue by measuring the dielectric constant of that body fluid and/or tissue. In an example, a wearable glucose-monitoring microwave sensor can measure changes in glucose levels in nearby body fluid and/or tissue by measuring changes in the dielectric constant of that body fluid and/or tissue. In an example, there can be an inverse relationship between the glucose level in body tissue and/or fluid and the dielectric constant of that body fluid and/or tissue.

In an example, a wearable glucose-monitoring microwave sensor can collect data concerning the amount and spectral distribution of microwave energy which is reflected from body tissue and/or fluid. This can depend on the angle at which energy is reflected as well as the permittivity of the body tissue and/or fluid. In an example, a wearable glucose-monitoring microwave sensor can measure changes in microwave energy that is reflected from body tissue and/or fluid as well as microwave energy that is transmitted through body tissue and/or fluid. In an example, changes in intra-body glucose levels can change the reflection coefficient as measured by a wearable glucose-monitoring microwave sensor. In an example, changes in intra-body glucose levels can change the resonant frequency of a resonator within a wearable glucose-monitoring microwave sensor. In an example, microwave energy emitted near body tissue and/or fluid interacts with that body tissue and/or fluid, dispersing energy into the transmitted energy and creating a measurable distorted output signal. This distorted output signal can be used to help estimate intra-body glucose levels.

In an example, a microwave energy emitter which is part of a wearable glucose-monitoring microwave sensor can receive microwave energy as well as emit microwave energy. In an example, a microwave energy emitter can be configured to measure microwave energy which is reflected from body tissue and/or fluid. In an example, changes in the amount and/or spectrum of microwave energy reflected from body tissue and/or fluid can be used to measure changes in intra-body glucose levels. In an example, a wearable glucose-monitoring microwave sensor can be a spectroscopy sensor which measures changes in the spectrum of electromagnetic energy caused by reflection of that energy from body tissue and/or fluid.

In an example, a wearable glucose-monitoring microwave sensor can be an impedance sensor. In an example, a wearable glucose-monitoring microwave sensor can estimate changes in glucose levels in body fluid and/or tissue by measuring changes in the impedance of that body fluid and/or tissue. In an example, a wearable glucose-monitoring microwave sensor can be a resistance sensor. In an example, a wearable glucose-monitoring microwave sensor can estimate changes in glucose levels in body fluid and/or tissue by measuring changes in the resistance of that body fluid and/or tissue. In an example, a wearable glucose-monitoring microwave sensor can be an inductance sensor. In an example, a wearable glucose-monitoring microwave sensor can estimate changes in glucose levels in body fluid and/or tissue by measuring changes in the inductance of that body fluid and/or tissue.

In an example, a wearable glucose-monitoring microwave sensor can be a capacitance sensor. In an example, a wearable glucose-monitoring microwave sensor can estimate changes in glucose levels in body fluid and/or tissue by measuring changes in the capacitance of that body fluid and/or tissue. In an example, a wearable glucose-monitoring microwave sensor can be a conductance sensor. In an example, a wearable glucose-monitoring microwave sensor can estimate changes in glucose levels in body fluid and/or tissue by measuring changes in the conductance of that body fluid and/or tissue. In an example, a wearable glucose-monitoring microwave sensor can be a conductivity sensor. In an example, a wearable glucose-monitoring microwave sensor can estimate changes in glucose levels in body fluid and/or tissue by measuring changes in the conductivity of that body fluid and/or tissue.

In an example, a wearable glucose-monitoring microwave sensor can be an impedance sensor. In an example, a wearable glucose-monitoring microwave sensor can collect data which is used to estimate changes in glucose levels in body fluid and/or tissue by collecting data on changes in the impedance of that body fluid and/or tissue. In an example, a wearable glucose-monitoring microwave sensor can be a resistance sensor. In an example, a wearable glucose-monitoring microwave sensor can collect data which is used to estimate changes in glucose levels in body fluid and/or tissue by collecting data on changes in the resistance of that body fluid and/or tissue. In an example, a wearable glucose-monitoring microwave sensor can be an inductance sensor. In an example, a wearable glucose-monitoring microwave sensor can collect data which is used to estimate changes in glucose levels in body fluid and/or tissue by collecting data on changes in the inductance of that body fluid and/or tissue.

In an example, a wearable glucose-monitoring microwave sensor can be a capacitance sensor. In an example, a wearable glucose-monitoring microwave sensor can collect data which is used to estimate changes in glucose levels in body fluid and/or tissue by collecting data on changes in the capacitance of that body fluid and/or tissue. In an example, a wearable glucose-monitoring microwave sensor can be a conductance sensor. In an example, a wearable glucose-monitoring microwave sensor can collect data which is used to estimate changes in glucose levels in body fluid and/or tissue by collecting data on changes in the conductance of that body fluid and/or tissue. In an example, a wearable glucose-monitoring microwave sensor can be a conductivity sensor. In an example, a wearable glucose-monitoring microwave sensor can collect data which is used to estimate changes in glucose levels in body fluid and/or tissue by collecting data on changes in the conductivity of that body fluid and/or tissue.

In an example, a wearable glucose-monitoring microwave sensor can comprise: a first component which is configured to emit microwave energy in proximity to body tissue and/or fluid; and a second component which receives this microwave energy after it has interacted with the body tissue and/or fluid. In an example, a wearable glucose-monitoring microwave sensor can comprise: a first component which is configured to transmit microwave energy into body tissue and/or fluid; and a second component which receives this microwave energy after it has been transmitted through the body tissue and/or fluid. In an example, a wearable glucose-monitoring microwave sensor can comprise: a first component which is configured to emit microwave energy in proximity to body tissue and/or fluid and to receive microwave energy which is reflected from this body tissue and/or fluid; and a second component which receives this microwave energy after it has been transmitted through the body tissue and/or fluid.

In an example, a wearable glucose-monitoring microwave sensor can comprise: a microwave energy emitter which is configured to emit microwave energy in proximity to body tissue and/or fluid; and a microwave energy receiver which receives this microwave energy after it has interacted with the body tissue and/or fluid. In an example, a wearable glucose-monitoring microwave sensor can comprise: a microwave energy emitter which is configured to transmit microwave energy into body tissue and/or fluid; and a microwave energy receiver which receives this microwave energy after it has been transmitted through the body tissue and/or fluid. In an example, a wearable glucose-monitoring microwave sensor can comprise: a microwave energy emitter which is configured to emit microwave energy in proximity to body tissue and/or fluid and to receive microwave energy which is reflected from this body tissue and/or fluid; and a microwave energy receiver which receives this microwave energy after it has been transmitted through the body tissue and/or fluid.

In an example, a wearable glucose-monitoring microwave sensor can be configured to measure microwave energy which has been transmitted across a distance in proximity to body tissue and/or fluid. In an example, changes in microwave energy transmitted through body tissue and/or fluid can be used to measure changes in intra-body glucose levels. In an example, a wearable glucose-monitoring microwave sensor can collect data which is used to measure intra-body glucose levels by transmitting electromagnetic energy through body tissue and/or fluid and receiving that electromagnetic energy after it has been transmitted through the body tissue and/or fluid. In an example, intra-body glucose levels can be estimated by analysis of: transmitted microwave energy received by a microwave energy receiver (which is affected by the electromagnetic properties of nearby body tissue and/or fluid): reflected microwave energy received by a microwave energy emitter (which is affected by the electromagnetic properties of nearby body tissue and/or fluid); or both.

In an example, a wearable glucose-monitoring microwave sensor can collect data which is used to measure intra-body glucose levels by emitting electromagnetic energy in proximity to body tissue and/or fluid and by receiving that electromagnetic energy after it has been reflected from the body tissue and/or fluid. In an example, intra-body glucose levels can be estimated by collecting data on microwave energy reflected from body tissue and/or fluid, collecting data on microwave energy transmitted through body tissue and/or fluid, or both.

In an example, a wearable glucose-monitoring microwave sensor can collect data concerning the interaction between an electromagnetic field and body tissue and/or fluid. In an example, intra-body glucose levels can be estimated by analysis of the permittivity or dielectric constant of nearby body tissue and/or fluid. In an example, a wearable glucose-monitoring microwave sensor can measure intra-body glucose levels by measuring how microwave energy emitted by a microwave energy emitter interacts with body tissue and/or fluid between the microwave energy emitter and a microwave energy receiver. In an example, a wearable glucose-monitoring microwave sensor can collect data which is used to measure intra-body glucose levels by creating an electromagnetic field which interacts with body tissue and/or fluid and by collecting data concerning the interaction between that electromagnetic field and the body tissue and/or fluid.

In an example, there can be a gap between a first (microwave-emitting) component and a second (microwave-receiving) component. In an example, there can be an insulator between a first (microwave-emitting) component and a second (microwave-receiving) component. In an example, there can be body tissue and/or fluid directly between a first (microwave-emitting) component and a second (microwave-receiving) component. In an example, there can be body tissue and/or fluid in proximity to a gap or insulator between a first (microwave-emitting) component and a second (microwave-receiving) component. In an example, a wearable glucose-monitoring microwave sensor can comprise: a microwave energy emitter and a microwave energy receiver, wherein the transmitter and receiver are separated and worn in proximity to body tissue and/or fluid.

In an example, a wearable glucose-monitoring microwave sensor can comprise a microwave energy emitting pole and a microwave energy receiving pole. In an example, a wearable glucose-monitoring microwave sensor can comprise a microwave energy emitting antenna and a microwave energy receiving antenna. In an example, a wearable glucose-monitoring microwave sensor can comprise a microwave energy emitting surface and a microwave energy receiving surface. In an example, a wearable glucose-monitoring microwave sensor can be selected from the group consisting of: defected ground structure, electromagnetic resonator, microwave antenna, microwave probe, microstrip, microwave spectrometer, and wave guide.

In an example, a wearable glucose-monitoring microwave sensor can comprise a microwave energy emitter and a microwave energy receiver which are co-planar. In an example, the microwave energy emitter and the microwave energy receiver can both lie within a common flat plane. In an example, a wearable glucose-monitoring microwave sensor can comprise a microwave energy emitter and a microwave energy receiver which are co-linear. In an example, the longitudinal axis of a microwave energy emitter and the longitudinal axis of a microwave energy receiver can be aligned along a common (virtual) straight line, with a gap or insulator between them. In an example, a wearable glucose-monitoring microwave sensor can comprise a microwave energy emitter and a microwave energy receiver which are parallel. In an example, the longitudinal axis of a microwave energy emitter can be parallel to the longitudinal axis of a microwave energy receiver. In an example, the ends of a microwave energy emitter and a microwave energy receiver which are closest to each other can be parallel. In an example, a microwave energy emitter and a microwave energy receiver can both be “T” shaped and in a symmetric configuration wherein their closest ends are parallel.

In an example, a wearable glucose-monitoring microwave sensor can comprise a microwave energy emitter and a microwave energy receiver whose closest edges are equidistant. In an example, the portions of the perimeter of a microwave energy emitter and the perimeter of a microwave energy receiver which are closest to each other can be separated by a constant distance. In an example, an equidistant configuration of a microwave energy emitter and microwave energy receiver can be an interlocking or interdigitating configuration. In an example, an equidistant configuration of a microwave energy emitter and microwave energy receiver can be a geometrically complementary configuration. In an example, an equidistant configuration of a microwave energy emitter and microwave energy receiver can be a sinusoidally-complementary configuration. In an example, a wearable glucose-monitoring microwave sensor can comprise two adjacent microwave antennae with interlocking, interdigitating, or intermeshing projections.

In an example, a wearable glucose-monitoring microwave sensor can comprise a microwave energy emitter and a microwave energy receiver which are both within the same curved planar surface. In an example, a wearable glucose-monitoring microwave sensor can comprise an arcuate microwave energy emitter and an arcuate microwave energy receiver which are configured to conform to the curved surface of a person's body. In an example, a wearable glucose-monitoring microwave sensor can comprise an arcuate microwave energy emitter and arcuate microwave energy receiver which are configured to be a uniform distance from the curved surface of a person's body. In an example, a wearable glucose-monitoring microwave sensor can comprise an arcuate microwave energy emitter and arcuate microwave energy receiver which are configured to be equidistant from the curved surface of a person's body. In an example, a wearable glucose-monitoring microwave sensor can comprise an arcuate microwave energy emitter and an arcuate microwave energy receiver which are contained within an arcuate circular band or ring which is worn around a portion of a person's body.

In an example, a wearable glucose-monitoring microwave sensor can comprise an arcuate microwave energy emitter and an arcuate microwave energy receiver which are co-arcuate, being located along the perimeter and/or surface of the same (virtual) circle, ellipse, oval, band, ring, or cylinder. In an example, a wearable glucose-monitoring microwave sensor can comprise an arcuate microwave energy emitter and an arcuate microwave energy receiver which are co-arcuate, being located along the same arc of a common (virtual) circle, ellipse, oval, band, ring, or cylinder. In an example, a wearable glucose-monitoring microwave sensor can comprise an arcuate microwave energy emitter and/or an arcuate microwave energy receiver with a saddle shape. In an example, a wearable glucose-monitoring microwave sensor can comprise an arcuate microwave energy emitter and/or an arcuate microwave energy receiver with a conic section shape.

In an example, a wearable glucose-monitoring microwave sensor can comprise a flexible microwave energy emitter and a flexible arcuate microwave energy receiver which are configured to conform to the curved surface of a person's body when worn on the person's body. In an example, a wearable glucose-monitoring microwave sensor can comprise an arcuate microwave energy emitter and an arcuate microwave energy receiver which are within a flexible, stretchable, and/or elastic wearable device which conforms to the curvature of the surface of a person's body. In an example, a wearable glucose-monitoring microwave sensor can comprise a microwave energy emitter and a microwave energy receiver which are within a flexible, stretchable, and/or elastic wearable device so as to keep the microwave energy emitter and microwave energy receiver a uniform distance from the surface of a person's body.

In an example, a wearable glucose-monitoring microwave sensor can comprise a spiral microwave antenna. In an example, a spiral microwave antenna can be flat. In an example, a spiral microwave antenna can be saddle-shaped. In an example, a spiral microwave antennae can be configured to conform to the curved surface of a person's body. In an example, a wearable glucose-monitoring microwave sensor can include a spiral microwave energy emitter. In an example, a wearable glucose-monitoring microwave sensor can include a spiral microwave energy receiver. In an example, a wearable glucose-monitoring microwave sensor can include two adjacent spiral antennae. In an example, a wearable glucose-monitoring microwave sensor can include two concentric and/or intertwined spiral antennae. In an example, a wearable glucose-monitoring microwave sensor can include two parallel and/or stacked spiral antennae. In an example, a wearable glucose-monitoring microwave sensor can include spiral antennae with opposite clockwise directions. In an example, a spiral microwave antenna can be a circular, oval, square, hexagonal, or octagonal spiral.

In an example, a wearable glucose-monitoring microwave sensor can comprise a microwave energy emitter and a microwave energy receiver which are nested and/or concentric. In an example, a wearable glucose-monitoring microwave sensor can comprise a microwave energy emitter and a microwave energy receiver comprising nested and/or concentric rings which form a target or “bulls eye” configuration. In an example, a microwave energy emitter can further comprise a plurality of nested and/or concentric microwave antennae. In an example, a microwave energy receiver can further comprise a plurality of nested and/or concentric microwave antennae.

In an example, a wearable glucose-monitoring microwave sensor can comprise an open-ended coaxial probe. In an example, a wearable glucose-monitoring microwave sensor can comprise an open-ended coaxial probe which is configured to be substantially perpendicular to the surface of a person's body. In an example, a wearable glucose-monitoring microwave sensor can comprise an open-ended coaxial probe which is configured to be substantially parallel to the surface of a person's body. In an example, a wearable glucose-monitoring microwave sensor can comprise a waveguide. In an example, a waveguide can have a longitudinal axis which is perpendicular to the surface of a person's body. In an example, a waveguide can have a longitudinal axis which is parallel to the surface of a person's body. In an example, a waveguide can be cylindrical, cubic, spherical, or rectangular.

In an example, a wearable glucose-monitoring microwave sensor can have a shape selected from the group consisting of: “bulls eye” pattern, arc of a circle, circle, circular spiral, coaxial probe, comb shape, concentric rings, conic section, convex lens shape, cube shape, cylinder, cylindrical band, dumbbell, ellipse, hemisphere, hexagon, H-shape antenna, microstrip, nested rings, non-equilateral polygon, octagon, Omega-shape, oval, oval spiral, parallel rings, polygon with rounded vertices, polygonal spiral, quadrilateral, rectangle, rectangular cylinder, rectangular spiral, ring, ring between two microwave poles, saddle shape, sawtooth shape, sinusoidal wave, sphere, split ring, square, square spiral, square wave, S-shape, stacked rings, straight co-linear poles, straight line, target pattern, triangle, T-shape, two complementary sinusoidal shapes, two intertwined spirals, two symmetric spirals, U-shape, Y-shape, and zigzag pattern.

In an example, a wearable glucose-monitoring microwave sensor can further comprise a microwave energy emitter with a shape selected from the group consisting of: “bulls eye” pattern, arc of a circle, circle, circular spiral, coaxial probe, comb shape, concentric rings, conic section, convex lens shape, cube shape, cylinder, cylindrical band, dumbbell, ellipse, hemisphere, hexagon, H-shape antenna, microstrip, nested rings, non-equilateral polygon, octagon, Omega-shape, oval, oval spiral, parallel rings, polygon with rounded vertices, polygonal spiral, quadrilateral, rectangle, rectangular cylinder, rectangular spiral, ring, ring between two microwave poles, saddle shape, sawtooth shape, sinusoidal wave, sphere, split ring, square, square spiral, square wave, S-shape, stacked rings, straight co-linear poles, straight line, target pattern, triangle, T-shape, two complementary sinusoidal shapes, two intertwined spirals, two symmetric spirals, U-shape, Y-shape, and zigzag pattern.

In an example, a wearable glucose-monitoring microwave sensor can further comprise a microwave energy receiver with a shape selected from the group consisting of: “bulls eye” pattern, arc of a circle, circle, circular spiral, coaxial probe, comb shape, concentric rings, conic section, convex lens shape, cube shape, cylinder, cylindrical band, dumbbell, ellipse, hemisphere, hexagon, H-shape antenna, microstrip, nested rings, non-equilateral polygon, octagon, Omega-shape, oval, oval spiral, parallel rings, polygon with rounded vertices, polygonal spiral, quadrilateral, rectangle, rectangular cylinder, rectangular spiral, ring, ring between two microwave poles, saddle shape, sawtooth shape, sinusoidal wave, sphere, split ring, square, square spiral, square wave, S-shape, stacked rings, straight co-linear poles, straight line, target pattern, triangle, T-shape, two complementary sinusoidal shapes, two intertwined spirals, two symmetric spirals, U-shape, Y-shape, and zigzag pattern.

In an example, a wearable glucose-monitoring microwave sensor can comprise a plurality of microwave energy emitters and/or receivers. In an example, a wearable glucose-monitoring microwave sensor can comprise an array, matrix, or series of microwave energy emitters and/or receivers. In an example, a wearable glucose-monitoring microwave sensor can comprise a co-planar array, matrix, or series of microwave energy emitters and/or receivers. In an example, a wearable glucose-monitoring microwave sensor can comprise a co-planar repeating array, matrix, or series of microwave energy emitters and/or receivers.

In an example, a wearable glucose-monitoring microwave sensor can comprise an arcuate array, matrix, or series of microwave energy emitters. In an example, a wearable glucose-monitoring microwave sensor can comprise an arcuate array, matrix, or series of microwave energy emitters around an arcuate band that is worn around a portion of a person's body. In an example, a wearable glucose-monitoring microwave sensor can comprise an arcuate array, matrix, or series of microwave energy receivers. In an example, a wearable glucose-monitoring microwave sensor can comprise an arcuate array, matrix, or series of microwave energy receivers around an arcuate band that is worn around a portion of a person's body.

In an example, a wearable glucose-monitoring microwave sensor can comprise an array, matrix, or series of microwave energy emitters and/or receivers of different sizes or configurations. In an example, a wearable glucose-monitoring microwave sensor can comprise an array, matrix, or series of microwave energy emitters and/or receivers with a selected progression of different sizes (e.g. smaller to larger), shapes (e.g. less arcuate to more arcuate), gaps (e.g. closer together to farther apart), material compositions, and/or rotations.

In an example, a wearable glucose-monitoring microwave sensor can comprise an array, matrix, or series of microwave antennae with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of wave guides with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of microwave spectrometers with a selected progression of different sizes (e.g. smaller to larger), shapes (e.g. less arcuate to more arcuate), gaps (e.g. closer together to farther apart), material compositions, and/or rotations.

In an example, a wearable glucose-monitoring microwave sensor can comprise an array, matrix, or series of defected ground structures with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of electromagnetic resonators with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of split ring resonators with a selected progression of different sizes (e.g. smaller to larger), shapes (e.g. less arcuate to more arcuate), gaps (e.g. closer together to farther apart), material compositions, and/or rotations.

In an example, a glucose monitoring and managing system can comprise: a plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a co-planar plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: an arcuate plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a cylindrical plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: an array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors. In an example, a glucose monitoring and managing system can comprise: a co-planar array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors.

In an example, a glucose monitoring and managing system can comprise: an arcuate array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors. In an example, a glucose monitoring and managing system can comprise: a cylindrical or ring-shaped array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors.

In an example, a glucose monitoring and managing system can comprise: a plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a co-planar plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: an arcuate plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a cylindrical plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: a first wearable glucose-monitoring microwave sensor which collects data which is used to measure a person's intra-body glucose levels; a second wearable glucose-monitoring microwave sensor which collects data which is used to measure a person's intra-body glucose levels; and a wearable or implanted pump which delivers a glucose-level-modifying substance into the person's body based on data collected by the plurality of wearable glucose-monitoring microwave sensors. In an example, the first and second wearable glucose-monitoring microwave sensors can be co-linear. In an example, the first and second wearable glucose-monitoring microwave sensors can be co-planar. In an example, the first and second wearable glucose-monitoring microwave sensors can be stacked and/or parallel to each other. In an example, first and second wearable glucose-monitoring microwave sensors can be located along the curved surface of a common (virtual) ring, band, or cylinder. In an example, first and second wearable glucose-monitoring microwave sensors can be located along the curved surface of a finger ring, wrist band, or arm band.

In an example, a glucose monitoring and managing system can comprise: a first wearable glucose-monitoring microwave sensor which collects data which is used to measure a person's intra-body glucose levels; a second wearable glucose-monitoring microwave sensor which collects data which is used to measure a person's intra-body glucose levels, wherein the first and second wearable glucose-monitoring microwave sensors are both located around the circumference of a finger ring, wrist band, bracelet, shirt cuff, or arm band; and a wearable or implanted pump which delivers a glucose-level-modifying substance into the person's body based on data collected by the plurality of wearable glucose-monitoring microwave sensors.

In an example, a glucose monitoring and managing system can comprise: an array, matrix, or series of wearable glucose-monitoring microwave sensors which collect data which is used to measure a person's intra-body glucose levels, wherein this array, matrix, or series of wearable glucose-monitoring microwave sensors is distributed around (a portion of) the circumference of a finger ring, wrist band, bracelet, shirt cuff, or arm band; and a wearable or implanted pump which delivers a glucose-level-modifying substance into the person's body based on data collected by the array, matrix, or series of wearable glucose-monitoring microwave sensors.

In an example, a wearable glucose-monitoring microwave sensor can comprise: a wearable microwave energy emitter; an array, matrix, or series of wearable microwave energy receivers which collect data which is used to measure a person's intra-body glucose levels; and a wearable or implanted pump which delivers a glucose-level-modifying substance into the person's body based on data collected by the plurality of wearable glucose-monitoring microwave sensors. In an example, a wearable glucose-monitoring microwave sensor can comprise: a wearable microwave energy emitter; an array, matrix, or series of wearable microwave energy receivers which collect data which is used to measure a person's intra-body glucose levels, wherein this array, matrix, or series of wearable microwave energy receivers is distributed around (a portion of) the circumference of a finger ring, wrist band, bracelet, shirt cuff, or arm band; and a wearable or implanted pump which delivers a glucose-level-modifying substance into the person's body based on data collected by the plurality of wearable glucose-monitoring microwave sensors.

In an example, a wearable glucose-monitoring microwave sensor can comprise: an array, matrix, or series of wearable microwave energy emitters, wherein this array, matrix, or series of wearable microwave energy emitters is distributed around (a portion of) the circumference of a finger ring, wrist band, bracelet, shirt cuff, or arm band; a wearable microwave energy receiver which collects data which is used to measure a person's intra-body glucose levels; and a wearable or implanted pump which delivers a glucose-level-modifying substance into the person's body based on data collected by the plurality of wearable glucose-monitoring microwave sensors.

In an example, a microwave energy emitter and a microwave energy receiver which comprise a wearable glucose-monitoring microwave sensor can be symmetric with respect to a line perpendicular to the shortest-line distance between them. In an example, a microwave energy emitter and a microwave energy receiver can be symmetric with respect to a line bisecting the gap between them. In an example, the ends of a microwave energy emitter and a microwave energy receiver which face each other can have the same shape. In an example, the ends of a microwave energy emitter and a microwave energy receiver which face each other can have complementary or interlocking shapes.

In an example, a plurality of wearable glucose-monitoring microwave sensors can be configured to confirm to the arcuate surface of a portion of a person's body. In an example, a plurality of wearable glucose-monitoring microwave sensors can be configured to encircle a person's finger, wrist, or arm. In an example, a plurality of wearable glucose-monitoring microwave sensors can be configured to span a portion of the circumference of a person's finger, wrist, or arm. In an example, an array, matrix, or series of wearable glucose-monitoring microwave sensors can be configured to span (a portion of) the circumference of a person's finger, wrist, or arm. In an example, a flexible array, matrix, or series of wearable glucose-monitoring microwave sensors can be configured to conform to (a portion of) the circumference of a person's finger, wrist, or arm.

In an example, a wearable glucose-monitoring microwave sensor can comprise a flexible array of microwave sensors which bends and/or stretches to conform to the arcuate surface of a portion of a person's body. In an example, a wearable glucose-monitoring microwave sensor can comprise a flexible array of microwave antennas which bends and/or stretches to conform to the arcuate surface of a portion of a person's body. In an example, a wearable glucose-monitoring microwave sensor can comprise a flexible array of wave guides which bends and/or stretches to conform to the arcuate surface of a portion of a person's body.

In an example, a wearable glucose-monitoring microwave sensor can further comprise one or more inflatable members whose inflation level can be adjusted to adjust the location of the sensor relative to the surface of a person's body. In an example, a wearable glucose-monitoring microwave sensor can further comprise one or more inflatable members whose inflation level can be adjusted to adjust the pressure of the sensor on the surface of a person's body. In an example, a wearable glucose-monitoring microwave sensor can further comprise one or more pneumatic or hydraulic members which can be adjusted to adjust the location of the sensor relative to the surface of a person's body. In an example, a wearable glucose-monitoring microwave sensor can further comprise one or more pneumatic or hydraulic members which can be adjusted to adjust the pressure of the sensor on the surface of a person's body.

In an example, a plurality of wearable glucose-monitoring sensors can be three-dimensionally stacked. In an example, a plurality of wearable glucose-monitoring sensors can be stacked in parallel to each other. In an example, a plurality of wearable glucose-monitoring sensors can be stacked along a virtual vector which extends radially outward from the cross-sectional center of a body member. In an example, an array, matrix, or series of wearable glucose-monitoring sensors can be three-dimensionally stacked. In an example, an array, matrix, or series of wearable glucose-monitoring sensors can be stacked in parallel to each other. In an example, an array, matrix, or series of wearable glucose-monitoring sensors can be stacked along a virtual vector which extends radially outward from the cross-sectional center of a body member.

In an example, a plurality of wearable glucose-monitoring sensors can be distributed within a three-dimensional array or matrix along two (polar) coordinate dimensions: radial compass degree or clock-hour position around the circumference of a body part; and distance from the cross-sectional center of the body part. In an example, a plurality of wearable glucose-monitoring sensors on a finger ring can be distributed within a three-dimensional array or matrix along two (polar) coordinate dimensions: radial compass degree or clock-hour position around the circumference of the finger; and distance from the cross-sectional center of the finger. In an example, a plurality of wearable glucose-monitoring sensors on a wrist band can be distributed within a three-dimensional array or matrix along two (polar) coordinate dimensions: radial compass degree or clock-hour position around the circumference of the wrist; and distance from the cross-sectional center of the wrist.

In an example, a stacked array, matrix, or series of wearable glucose-monitoring microwave sensors can have a selected progression of different sizes, shapes, and/or orientations along a vector extending outward from the cross-sectional center of a body part such as a finger, wrist, or arm. In an example, sensors closer to the surface of the finger, wrist, or arm can have a first size, shape, and/or orientation and sensors further from the surface of the finger, wrist, or arm can have a second size, shape, and/or orientation. In an example, a stacked array, matrix, or series of wearable glucose-monitoring microwave sensors can have a selected progression of different sizes, shapes, and/or orientations around the circumference of a body part such as a finger, wrist, or arm. In an example, sensors closer to a ventral surface of the finger, wrist, or arm can have a first size, shape, and/or orientation and sensors further from the ventral surface of the finger, wrist, or arm can have a second size, shape, and/or orientation.

In an example, one or more aspects of the configuration and/or operation of a wearable glucose-monitoring microwave sensor can be automatically adjusted to improve measurement of intra-body glucose levels. In an example, a wearable glucose-monitoring microwave sensor can further comprise one or more actuators which automatically adjust its location, orientation, position, and/or configuration in order to more accurately and consistently measure intra-body glucose levels. In an example, such automatic adjustment occurs when a person first wears the wearable glucose-monitoring microwave sensor as the sensor is calibrated to the anatomy and/or physiology of that specific person. In an example, such automatic adjustment occurs each time the person wears the wearable glucose-monitoring microwave sensor as the sensor is calibrated to the specific placement of the device at different occasions. In an example, such automatic adjustment occurs in real time whenever the person is wearing the device to continually adjust for real-time body motion, sensor shifting, and other variables.

In an example, one or more actuators which automatically adjust the configuration and/or operation of a wearable glucose-monitoring microwave sensor can be electromagnetic actuators. In an example, one or more actuators which automatically adjust the configuration and/or operation of a wearable glucose-monitoring microwave sensor can be MEMS actuators. In an example, one or more actuators which automatically adjust the configuration and/or operation of a wearable glucose-monitoring microwave sensor can be pneumatic actuators. In an example, one or more actuators which automatically adjust the configuration and/or operation of a wearable glucose-monitoring microwave sensor can be hydraulic actuators.

In an example, an actuator can automatically adjust the distance between a wearable glucose-monitoring microwave sensor and the surface of a person's body in order to more accurately and/or consistently measure intra-body glucose levels. In an example, an actuator can automatically adjust the distance between a wearable glucose-monitoring microwave sensor and the surface of a person's body in order to maintain a relatively constant distance for more accurate and/or consistent measurement of intra-body glucose levels. In an example, an actuator can automatically adjust the distance between a wearable glucose-monitoring microwave sensor and the surface of a person's body when this distance would otherwise be changed by body motion and sensor shifting.

In an example, an actuator can automatically adjust the orientation of a wearable glucose-monitoring microwave sensor relative to the surface of a person's body in order to more accurately and/or consistently measure intra-body glucose levels. In an example, an actuator can automatically adjust the orientation of a wearable glucose-monitoring microwave sensor relative to the surface of a person's body in order to maintain a constant energy reflection angle for more accurate and/or consistent measurement of intra-body glucose levels. In an example, an actuator can automatically adjust the orientation of a wearable glucose-monitoring microwave sensor relative to the surface of a person's body when this orientation would otherwise be changed by body motion and sensor shifting.

In an example, an actuator can automatically adjust the position of a wearable glucose-monitoring microwave sensor relative to specific anatomical structures and/or landmarks on a person's body. In an example, an actuator can automatically adjust the position of a wearable glucose-monitoring microwave sensor to maintain proximity to specific anatomical structures and/or landmarks (such as specific portions of body vasculature) for more accurate and/or consistent measurement of intra-body glucose levels. In an example, an actuator can automatically adjust the location of a wearable glucose-monitoring microwave sensor relative to anatomical landmarks of a person's body when this location would otherwise be changed by body motion and sensor shifting. In an example, the location of a wearable glucose-monitoring microwave sensor can be automatically adjusted by one or more actuators in order to position it at the same location on a person's body when its location would otherwise be shifted by movement of the person's body.

In an example, a wearable glucose-monitoring microwave sensor can further comprise one or more actuators which automatically adjust the gap between a microwave energy emitter and a microwave energy receiver. In an example, the size of this gap can be adjusted when a person first wears the sensor in order to calibrate it to the specific anatomy and physiology of that person. In an example, the size of this gap can be adjusted each time a person wears the device in order to calibrate it for differences in placement or other variables from one wearing to the next. In an example, the size of this gap can be adjusted in real time in order to maintain accurate measurement of intra-body glucose levels. In an example, the gap between a microwave energy emitter and a microwave energy receiver can be automatically adjusted based on one or more parameters selected from the group consisting of: changes in the location on a person's body on which they are worn; changes their distance from the surface of the person's body; changes in the pressure and/or force which they apply to the person's body; changes in their angle and/or orientation relative to the person's body; changes in body temperature and/or ambient temperature; changes in body moisture level and/or ambient humidity; and body motion and/or speed.

In an example, a wearable glucose-monitoring microwave sensor can further comprise one or more actuators which automatically adjust the shape of the sensor. In an example, the shape of a sensor can be adjusted when a person first wears the sensor in order to calibrate it to the specific anatomy and physiology of that person. In an example, the shape of a sensor can be adjusted each time a person wears the device in order to calibrate it for differences in placement or other variables from one wearing to the next. In an example, the shape of a sensor can be adjusted in real time in order to maintain accurate measurement of intra-body glucose levels. In an example, the shape of a sensor can be automatically adjusted based on one or more parameters selected from the group consisting of: changes in the location on a person's body on which they are worn; changes their distance from the surface of the person's body; changes in the pressure and/or force which they apply to the person's body; changes in their angle and/or orientation relative to the person's body; changes in body temperature and/or ambient temperature; changes in body moisture level and/or ambient humidity; and body motion and/or speed.

In an example, a wearable glucose-monitoring microwave sensor can have a first level of operation and a second level of operation, wherein the second level of operation provides more accurate measurement of intra-body glucose levels than the first level of operation, and wherein the sensor can be reversibly changed from the first level of operation to the second level of operation. In an example, the second level of operation requires more power than the first level of operation. In an example, the second level of operation activates a larger number of sensors in a sensor array than the first level of operation. In an example, the second level of operation involves more frequent scans than the first level of operation.

In an example, operation of the wearable glucose-monitoring microwave sensor at the second level of operation can require more power and be more accurate than operation of the wearable glucose-monitoring microwave sensor at the first level of operation. In an example, automatic and reversible adjustment of the operation of a wearable glucose-monitoring microwave sensor from a first level of operation to a second level of operation, and vice versa, can help to conserve energy because the microwave sensor only operates at higher power levels when needed. In an example, automatic and reversible adjustment of the operation of a wearable glucose-monitoring microwave sensor from a first level of operation to a second level of operation, and vice versa, can help to reduce a person's exposure to microwave energy because the microwave sensor only operates at higher power levels when needed.

In an example, a wearable glucose-monitoring microwave sensor can be changed from a first level of operation to a second level of operation based on changes in physiological or environmental factors which are detected by other wearable or implanted sensors. In an example, a wearable glucose-monitoring microwave sensor can be changed from a first level of operation to a second level of operation when analysis of data collected by other wearable or implanted sensors indicates that a person is eating or likely to eat soon. In an example, a wearable glucose-monitoring microwave sensor can be changed from a first level of operation to a second level of operation when data indicates a glucose level below or above a normal range. In an example, a wearable glucose-monitoring microwave sensor can be changed from a first level of operation to a second level of operation when analysis of data collected by other wearable or implanted sensors indicates that a person very physically active. In an example, a wearable glucose-monitoring microwave sensor can operate at a first level of operation until an abnormally low or high intra-body glucose level is detected, at which time it can shift to operation at a second level of operation.

In an example, a wearable glucose-monitoring microwave sensor can be changed from a first level of operation to a second level of operation based on one or more factors selected from the group consisting of: detection that a person is eating; detection of an abnormal intra-body glucose level; a change in the position and/or orientation of a microwave sensor relative to a person's body; a change in the degree of contact and/or pressure between a microwave sensor and a person's body; a change in a person's body moisture level and/or environmental moisture level; a change in a person's body temperature and/or environmental temperature; a change in ambient electromagnetic energy level; a change in a person's body movement speed, acceleration, orientation, or direction; and a change in geographic location (e.g. detected by GPS) and/or proximity to a place that is associated with food consumption.

In an example, a wearable glucose-monitoring microwave sensor can emit electromagnetic energy in the range of 100 MHz to 100 GHz. In an example, a wearable glucose-monitoring microwave sensor can emit electromagnetic energy in the range of 100 MHz to 5 GHz. In an example, a wearable glucose-monitoring microwave sensor can emit electromagnetic energy in the range of 300 MHz to 300 GHz. In an example, a wearable glucose-monitoring microwave sensor can emit microwave energy at a constant frequency. In an example, a wearable glucose-monitoring microwave sensor can emit microwave energy at varying frequencies within a selected range in order to collect information about the interaction between that energy and body tissue across a spectrum of frequencies. In an example, a wearable glucose-monitoring microwave sensor can emit microwave energy at frequencies which sweep through a selected range.

In an example, a wearable glucose-monitoring microwave sensor can be a microwave spectroscopy sensor. In an example, a microwave spectroscopy sensor can measure the permittivity of body tissue and/or fluid as a function of microwave energy at varying frequencies. In an example, a microwave spectroscopy sensor can measure multiple resonant frequencies of one or more resonators by sweeping through a range of microwave frequencies. In an example, a wearable glucose-monitoring microwave sensor can be a spectroscopy sensor which collects information about electromagnetic interaction between microwave energy and body tissue and/or fluids at multiple microwave frequencies. In an example, information about electromagnetic interaction throughout a microwave frequency spectrum can provide more accurate information concerning intra-body glucose levels than information about electromagnetic interaction at a single microwave frequency. In an example, a wearable glucose-monitoring microwave sensor can be a microwave spectrometer. In an example, a wearable glucose-monitoring microwave sensor can be an impedance spectroscopy sensor.

In an example, a wearable glucose-monitoring microwave sensor can include a microwave emitter which sweeps through a range of microwave frequencies. In an example, a wearable glucose-monitoring microwave sensor can include a microwave-emitting component which emits pulses of ultra-wideband microwave energy. In an example, a wearable glucose-monitoring microwave sensor can expose body tissue and/or fluid to microwave energy at different frequencies and collect information on the response of that body tissue and/of fluid at each frequency. In an example, a wearable glucose-monitoring microwave sensor can measure intra-body glucose levels via impedance or dielectric spectroscopy.

In an example, a glucose monitoring and managing system can further comprise an electromagnetic resonator. In an example, a wearable glucose-monitoring microwave sensor can further comprise an electromagnetic resonator. One or more resonant frequencies of such a resonator can change with changes in the glucose level of nearby body tissue and/or body fluid. One or more resonant frequencies of such a resonator can be changed by a change in the permittivity of nearby body tissue and/or fluid which, in turn, can be changed by changes in the glucose level of nearby body tissue and/or body fluid. The resonant frequency of a resonator is reduced by dielectric loading of body tissue. In an example, there can be an inverse relationship between the glucose levels of body tissue and/or fluid and the resonant frequency of a resonator. In this manner, intra-body glucose levels can be estimated by measuring one or more resonant frequencies of an electromagnetic resonator.

In an example, an electromagnetic resonator can be located between a microwave energy emitter and a microwave energy receiver. In an example, when the resonator resonates at a resonant frequency, the resonator acts as a partial short-circuit between the microwave energy emitter and the microwave energy receiver. In an example, a resonator creates a drop in impedance in the transmission of microwave energy from the transmitter to the receiver when the resonator resonates. In this manner, one or more resonant frequencies of the resonator can be observed by observing changes in the transmission of microwave energy from the microwave energy emitter to the microwave energy receiver. In an example, multiple resonant frequencies can be observed with a selected range (or spectrum) of microwave energy frequencies.

In an example, a wearable glucose-monitoring microwave sensor can comprise a microwave energy emitter, a microwave energy receiver, and an electromagnetic resonator, wherein one or more resonant frequencies of the resonator are shifted by changes in the glucose levels of nearby body tissue and/or fluid. In an example, changes in the glucose levels of nearby body tissue and/or fluid change the permittivity of the body tissue and/or fluid which, in turn, changes the one or more resonant frequencies of the resonator. In an example, a wearable glucose-monitoring microwave sensor can comprise a microwave energy emitter, a microwave energy receiver, and a resonator, wherein changes in microwave transmission are used to measure changes in the glucose levels of nearby body tissue and/or fluid.

In an example, a wearable glucose-monitoring microwave sensor can comprise: a first component which transmits microwave energy; a second component which receives microwave energy; and a third component between the first and second components which resonates in response to microwave energy transmission. In an example, the resonant frequency of the third component changes based on changes in the glucose levels of nearby body tissue and/or fluid. In an example, the third component can be a microwave resonator. In an example, the resonant frequency of the third component changes based on changes in the permittivity of nearby body tissue and/or fluid which, in turn, depends on the glucose levels in that body tissue and/or fluid.

An electromagnetic resonator does not have to be in direct contact with a person's body in order to have its resonant frequency affected by the electromagnetic properties of body tissue and/or fluid. An electromagnetic field can spill out into nearby body tissue and/or fluid from the side of a resonator which faces the body tissue and/or fluid. This electromagnetic field can penetrate nearby body tissue and/or fluid and measure glucose levels at a greater tissue depth than is possible with measurement methods which rely on reflection of light energy. In an example, changes in the glucose levels of nearby body tissue and/or fluid change the permittivity of that body tissue and/or fluid which change the resonating frequency of the electromagnetic resonator. In this manner, observed changes in one or more resonant frequencies of the resonator can be used to estimate intra-body glucose levels.

In an example, a resonator which is part of a wearable glucose-monitoring microwave sensor can be arcuate. In an example, a resonator which is part of a wearable glucose-monitoring microwave sensor can be circular. In an example, a resonator which is part of a wearable glucose-monitoring microwave sensor can be a ring. In an example, a resonator which is part of a wearable glucose-monitoring microwave sensor can have a shape which is selected from the group consisting of: circular ring or band; concentric ring; target pattern; nested and/or concentric rings; ring with a single split or gap; ring with multiple splits (or gaps); and three-dimensionally stacked or parallel rings. In an example, a resonator which is part of a wearable glucose-monitoring microwave sensor can have a shape which is selected from the group consisting of: cylinder; cylindrical band; ellipse; elliptical ring; and oval.

In an example, a resonator which is part of a wearable glucose-monitoring microwave sensor can be polygonal. In an example, a resonator which is part of a wearable glucose-monitoring microwave sensor can be square or rectangular. In an example, a resonator which is part of a wearable glucose-monitoring microwave sensor can have a shape which is selected from the group consisting of: polygon with rounded vertices; square, rectangle, or other quadrilateral; square or rectangular cylinder; triangle; hexagon or octagon; non-equilateral polygon; other polygon or polygon with rounded vertexes; cube; and stacked or parallel squares.

In an example, a resonator which is part of a wearable glucose-monitoring microwave sensor can be a spiral. In an example, a resonator which is part of a wearable glucose-monitoring microwave sensor can be selected from the group consisting of: circular spiral; oval spiral; square spiral; rectangular spiral; other polygonal spiral; two intertwined spirals; two symmetric spirals; and two stacked spirals. In various examples, a resonator can be selected from the group consisting of: annular Bragg resonator, bow tie shape, co-linear elements, comb shape, conic section shape, convex lens shape, C-shape, dumbbell shape, figure eight shape, hemisphere, H-shape, microstrip, Omega-shape, saddle shape, sawtooth shape, semicircle, sinusoidal shape, sphere, square wave shape, S-shape, straight line, T-shape, U-shape, Y-shape, and zigzag shape.

In an example, a resonator which is part of a wearable glucose-monitoring microwave sensor can have one or more splits (or gaps). In an example, a resonator which is part of a wearable glucose-monitoring microwave sensor can have one or more splits (or gaps) in its perimeter. The capacitance of a split or gap in the perimeter of a resonator can cause a resonator to resonate at a wave length which is larger than the size of the resonator. In an example, a resonator can have a gap in its perimeter which produces large capacitance values and lowers the resonant frequency of the resonator. In an example, a resonator can be a ring with one or more spits or gaps in its circumference. In an example, a resonator can be a Split Ring Resonator (SRR). In an example, a resonator can comprise two or more nested and/or concentric rings with spits or gaps in their circumferences.

In an example, a wearable glucose-monitoring microwave sensor can comprise: a microwave energy emitter; a microwave energy receiver; and a Split Ring Resonator (SRR) between the microwave energy emitter and the microwave energy receiver. In an example, changes in the glucose levels of nearby body tissue and/or fluid change the permittivity of that body tissue and/or fluid, which changes the resonant frequency of the Split Ring Resonator (SRR), which enables measurement of intra-body glucose levels.

In an example, compass or clock-hour coordinates for a resonator can be defined with respect to a “connector line” between a microwave energy emitter and a microwave energy receiver. In an example, the “connector line” can be defined as a virtual straight line which is drawn between the closest points on the microwave energy emitter and the microwave energy receiver. In an alternative example, the “connector line” can be defined as a virtual straight line which is drawn between the centroids of a microwave energy emitter and microwave energy receiver. In an example, virtual compass or clock-hour coordinates can be superimposed on a resonator, centered around the centroid of the resonator, so that the connector line intersects the 270-degree (9 o'clock) and the 90-degree (3 o'clock) locations on the compass or clock-hour coordinate system. In an example, virtual compass or clock-hour coordinates can be centered around the mid-point of the “connector line.”

In an example, a single split or gap of a resonator can be centered at the 360-degree (12 o'clock) or 180-degree (6 o'clock) location using the above-defined compass or clock-hour coordinates. In an example, there can be two splits (or gaps) in a resonator, one centered at the 360-degree (12 o'clock) location and one at the 180-degree (6 o'clock) location. More generally, a resonator can have a number (X) of perimeter splits of gaps whose positions are separated by (360/X) degrees.

In an example, with nested and/or concentric resonators, the location of the split or gap in an outer element (e.g. ring or square) can be rotated 180 degrees relative to the location of the split or gap in the inner element (e.g. ring or square). More generally, a resonator can have a number (Y) of nested and/or concentric rings, wherein the locations of splits (or gaps) in pairs of adjacent rings (e.g. proximal rings separated by an inter-ring gap) vary from each other by 180-degree rotation. More generally, a resonator can have (Y) nested and/or concentric rings, each with (X) splits (or gaps), wherein the locations of splits (or gaps) in pairs of adjacent rings (e.g. proximal rings separated by an inter-ring gap) vary from each other by (360/2X) degrees.

In an example, a wearable glucose-monitoring microwave sensor can comprise a first resonator with a first resonant frequency and a second resonator with a second resonant frequency. In an example, a first resonator can differ from a second resonator in one or more aspects selected from the group consisting of: resonator shape; resonator size; resonator orientation with respect to the connector line; resonator material; resonator distance from the surface of a person's body; resonator symmetry; number of splits (or gaps); size of splits (or gaps); location and/or orientation of splits (or gaps); nested configuration; and stacked configuration. In an example, a wearable glucose-monitoring microwave sensor can comprise a plurality of resonators with different resonant frequencies. In an example, a plurality of resonators with different resonant frequencies can provide more rapid and/or complete information on the permittivity of nearby body tissue and/or fluid (and thus intra-body glucose levels) across a selected range of microwave frequencies than is possible with a single resonator.

In an example, a wearable glucose-monitoring microwave sensor can comprise a first resonator with a first shape and a second resonator with a second shape, wherein the second shape is different than the first shape. In an example, a wearable glucose-monitoring microwave sensor can comprise a first resonator with a first shape and a second resonator with a second shape, wherein the second shape is more arcuate than the first shape. In an example, a wearable glucose-monitoring microwave sensor can comprise a first resonator with a first shape and a second resonator with a second shape, wherein the second shape is more convex than the first shape. In an example, a wearable glucose-monitoring microwave sensor can comprise a first resonator with a first size and a second resonator with a second size, wherein the second size is at least 10% larger than the first size.

In an example, a wearable glucose-monitoring microwave sensor can comprise a first resonator with a first orientation and a second resonator with a second orientation, wherein the second orientation is different than the first orientation. In an example, a wearable glucose-monitoring microwave sensor can comprise a first resonator with a first orientation and a second resonator with a second orientation, wherein the second orientation is rotated by at least 10 degrees relative to the first orientation. In an example, a wearable glucose-monitoring microwave sensor can comprise a first resonator with a split (or gap) with a first position relative to compass coordinates and a second resonator with a split (or gap) with a first position relative to compass coordinates, wherein the second position is rotated by at least 10 degrees relative to the first position.

In an example, a wearable glucose-monitoring microwave sensor can comprise a first resonator with a first number of splits (or gaps) and a second resonator with a second number of splits (or gaps), wherein the second number is larger than the first number. In an example, a wearable glucose-monitoring microwave sensor can comprise a first resonator made from a first material and a second resonator made from a second material, wherein the second material is different than the first material. In an example, a wearable glucose-monitoring microwave sensor can comprise a first resonator made from a first material and a second resonator made from a second material, wherein the second material is more conductive from the first material. In an example, a wearable glucose-monitoring microwave sensor can comprise a first resonator located at a first distance from the surface of a person's body and a second resonator located at a second distance from the surface of a person's body, wherein the second distance is greater than the first distance.

In an example, a wearable glucose-monitoring microwave sensor can comprise a first resonator and a second resonator, wherein the first and second resonators are co-planar. In an example, a wearable glucose-monitoring microwave sensor can comprise a first resonator and a second resonator, wherein the first and second resonators both lie in the same flat plane. In an example, a first resonator and a second resonator can be co-planar and adjacent. In an example, a first resonator and a second resonator can be co-planar and symmetric. In an example, a first resonator and a second resonator can be co-planar and repeated. In an example, a first resonator and a second resonator can be co-planar and nested and/or concentric. In an example, a wearable glucose-monitoring microwave sensor can comprise an array, matrix, or series of co-planar resonators. In an example, a wearable glucose-monitoring microwave sensor can comprise an array, matrix, or series of co-planar resonators with different resonant frequencies.

In an example, a wearable glucose-monitoring microwave sensor can comprise a first Split Ring Resonator (SRR) and a second Split Ring Resonator (SRR), wherein the first and second Split Ring Resonators (SRRs) are co-planar. In an example, a wearable glucose-monitoring microwave sensor can comprise a first Split Ring Resonator (SRR) and a second Split Ring Resonator (SRR), wherein the first and second Split Ring Resonators (SRRs) both lie in the same flat plane. In an example, a first Split Ring Resonator (SRR) and a second Split Ring Resonator (SRR) can be co-planar and adjacent. In an example, a first Split Ring Resonator (SRR) and a second Split Ring Resonator (SRR) can be co-planar and symmetric. In an example, a first Split Ring Resonator (SRR) and a second Split Ring Resonator (SRR) can be co-planar and repeated. In an example, a first Split Ring Resonator (SRR) and a second Split Ring Resonator (SRR) can be co-planar and nested and/or concentric. In an example, a wearable glucose-monitoring microwave sensor can comprise an array, matrix, or series of co-planar Split Ring Resonators (SRRs). In an example, a wearable glucose-monitoring microwave sensor can comprise an array, matrix, or series of co-planar Split Ring Resonators (SRRs) with different resonant frequencies.

In an example, a wearable glucose-monitoring microwave sensor can comprise a uniform array, matrix, or series of identical co-planar resonators. In an example, a wearable glucose-monitoring microwave sensor can comprise a non-uniform array, matrix, or series of co-planar resonators. In an example, a wearable glucose-monitoring microwave sensor can comprise an array, matrix, or series of co-planar resonators, wherein there is a selected progression in resonator frequency along an axis of the array, matrix, or series. In an example, a wearable glucose-monitoring microwave sensor can comprise an array, matrix, or series of co-planar resonators, wherein there is a selected progression in resonator frequency from a lower resonant frequency to a higher resonant frequency along an axis of the array, matrix, or series. In an example, a wearable glucose-monitoring microwave sensor can comprise an array, matrix, or series of co-planar resonators, wherein there is a selected progression in resonator size along an axis of the array, matrix, or series. In an example, a wearable glucose-monitoring microwave sensor can comprise an array, matrix, or series of co-planar resonators, wherein there is a selected progression in resonator size from a smaller to larger along an axis of the array, matrix, or series.

In an example, a wearable glucose-monitoring microwave sensor can comprise an arcuate array, matrix, or series of resonators. In an example, a wearable glucose-monitoring microwave sensor can comprise an array, matrix, or series of resonators which lie along the surface of a (virtual) ring or cylinder. In an example, a wearable glucose-monitoring microwave sensor can comprise an array, matrix, or series of resonators which lie along the surface of a finger ring, wrist band, or arm band. In an example, a wearable glucose-monitoring microwave sensor can comprise an array, matrix, or series of resonators which are distributed around (a portion of) the circumference of a finger ring, wrist band, or arm band. In an example, a wearable glucose-monitoring microwave sensor can comprise a cylindrical array, matrix, or series of resonators. In an example, a wearable glucose-monitoring microwave sensor can comprise an oval-shaped array, matrix, or series of resonators. In an example, a wearable glucose-monitoring microwave sensor can comprise a saddle-shaped array, matrix, or series of resonators.

In an example, a wearable glucose-monitoring microwave sensor can comprise a flexible, stretchable, and/or elastic arcuate array, matrix, or series of resonators which can bend to conform to the curvature of the surface of a person's body. In an example, a wearable glucose-monitoring microwave sensor can comprise a flexible, stretchable, and/or elastic array, matrix, or series of resonators which can conform to a relatively-uniform distance from the surface of a person's body. In an example, a wearable glucose-monitoring microwave sensor can comprise a flexible, stretchable, and/or elastic array, matrix, or series of resonators which can be incorporated into a wrist band or arm band. In an example, a wearable glucose-monitoring microwave sensor can comprise a flexible arcuate array, matrix, or series of resonators which can be incorporated into a shirt sleeve or cuff. In an example, a wearable glucose-monitoring microwave sensor can comprise a flexible arcuate array, matrix, or series of resonators which can be incorporated into the cuff of a pair of shorts or pants. In an example, a wearable glucose-monitoring microwave sensor can comprise a flexible arcuate array, matrix, or series of resonators which can be incorporated into a sock.

In an example, a wearable glucose-monitoring microwave sensor can comprise a plurality of stacked resonators. In an example, a wearable glucose-monitoring microwave sensor can comprise a plurality of three-dimensionally-stacked resonators. In an example, a wearable glucose-monitoring microwave sensor can comprise a plurality of parallel stacked resonators. In an example, a wearable glucose-monitoring microwave sensor can comprise a plurality of resonators which are stacked along a radial vector which extends outward from the cross-sectional center of a person's finger, wrist, arm, or leg. In an example, a wearable glucose-monitoring microwave sensor can comprise a plurality of resonators which are stacked along a vector which is perpendicular to the surface of a person's body. In an example, a wearable glucose-monitoring microwave sensor for estimating intra-body glucose levels can include stacked or parallel resonators. In an example, there can be two stacked or parallel resonators between a microwave energy emitter and a microwave energy receiver. In an example, there can be three of more stacked or parallel resonators between a microwave energy emitter and a microwave energy receiver.

In an example, a wearable glucose-monitoring microwave sensor can comprise a three-dimensional array, matrix, or series of resonators. In an example, a wearable glucose-monitoring microwave sensor can comprise a three-dimensional array, matrix, or series of resonators wherein at least one dimension of the array, matrix, or series is aligned with a vector which extends radially outward from the cross-sectional center of a person's finger, wrist, arm, or leg. In an example, a wearable glucose-monitoring microwave sensor can comprise a three-dimensional array, matrix, or series of resonators wherein at least one dimension of the array, matrix, or series is aligned with a vector which is perpendicular to the surface of a person's body. In an example, a wearable glucose-monitoring microwave sensor can comprise: a microwave energy emitter; a microwave energy receiver; and a three-dimensional array, matrix, or series of resonators between the microwave energy emitter and the microwave energy receiver.

In an example, a wearable glucose-monitoring microwave sensor can comprise a first resonator whose centroid is a first distance from the surface of a person's body and a second resonator whose centroid is a second distance from the surface of a person's body, wherein the second distance is greater than the first distance. In an example, a wearable glucose-monitoring microwave sensor can comprise a first resonator whose closest point to the surface of a person's body is a first distance and a second resonator whose closest point to the surface of a person's body is a second distance, wherein the second distance is greater than the first distance.

In an example, a wearable glucose-monitoring microwave sensor can comprise a plurality of stacked Split Ring Resonators (SRRs). In an example, a wearable glucose-monitoring microwave sensor can comprise a plurality of three-dimensionally-stacked Split Ring Resonators (SRRs). In an example, a wearable glucose-monitoring microwave sensor can comprise a plurality of parallel stacked Split Ring Resonators (SRRs). In an example, a wearable glucose-monitoring microwave sensor can comprise a plurality of Split Ring Resonators (SRRs) which are stacked along a radial vector which extends outward from the cross-sectional center of a person's finger, wrist, arm, or leg. In an example, a wearable glucose-monitoring microwave sensor can comprise a plurality of Split Ring Resonators (SRRs) which are stacked along a vector which is perpendicular to the surface of a person's body. In an example, a wearable glucose-monitoring microwave sensor for estimating intra-body glucose levels can include stacked or parallel Split Ring Resonators (SRRs). In an example, there can be two stacked or parallel Split Ring Resonators (SRRs) between a microwave energy emitter and a microwave energy receiver. In an example, there can be three of more stacked or parallel Split Ring Resonators (SRRs) between a microwave energy emitter and a microwave energy receiver.

In an example, a wearable glucose-monitoring microwave sensor can comprise a three-dimensional array, matrix, or series of Split Ring Resonators (SRRs). In an example, a wearable glucose-monitoring microwave sensor can comprise a three-dimensional array, matrix, or series of Split Ring Resonators (SRRs) wherein at least one dimension of the array, matrix, or series is aligned with a vector which extends radially outward from the cross-sectional center of a person's finger, wrist, arm, or leg. In an example, a wearable glucose-monitoring microwave sensor can comprise a three-dimensional array, matrix, or series of Split Ring Resonators (SRRs) wherein at least one dimension of the array, matrix, or series is aligned with a vector which is perpendicular to the surface of a person's body. In an example, a wearable glucose-monitoring microwave sensor can comprise: a microwave energy emitter; a microwave energy receiver; and a three-dimensional array, matrix, or series of Split Ring Resonators (SRRs) between the microwave energy emitter and the microwave energy receiver.

In an example, a wearable glucose-monitoring microwave sensor can comprise a first Split Ring Resonator (SRR) whose centroid is a first distance from the surface of a person's body and a second Split Ring Resonator (SRR) whose centroid is a second distance from the surface of a person's body, wherein the second distance is greater than the first distance. In an example, a wearable glucose-monitoring microwave sensor can comprise a first Split Ring Resonator (SRR) whose closest point to the surface of a person's body is a first distance and a second Split Ring Resonator (SRR) whose closest point to the surface of a person's body is a second distance, wherein the second distance is greater than the first distance.

In an example, a wearable glucose-monitoring microwave sensor can comprise a uniform three-dimensional array, matrix, or series of resonators. In an example, a wearable glucose-monitoring microwave sensor can comprise a non-uniform three-dimensional array, matrix, or series of resonators. In an example, resonators in a three-dimensional array, matrix, or series of resonators can vary in size, shape, resonant frequency, or other features. In an example, resonators in a three-dimensional array, matrix, or series which are closer to the surface of a person's body can be smaller and resonators which are farther from the surface of the person's body can be larger, or vice versa. In an example, resonators in a three-dimensional array, matrix, or series which are closer to the surface of a person's body can have a lower resonant frequency and resonators which are farther from the surface of the person's body can have a higher resonant frequency, or vice versa.

In an example, a wearable glucose-monitoring microwave sensor can comprise a first resonator with a first size which is configured to be worn a first distance from the surface of a person's body and a second resonator with a second size which is configured to be worn a second distance from the surface of a person's body, wherein the second size is greater than the first size and wherein the second distance is greater than the first distance. In an example, a wearable glucose-monitoring microwave sensor can comprise a first resonator with a first size which is configured to be worn a first distance from the surface of a person's body and a second resonator with a second size which is configured to be worn a second distance from the surface of a person's body, wherein the second size is at least 10% greater than the first size and wherein the second distance is at least 10% greater than the first distance.

In an example, one or more parameters of a resonator in a wearable glucose-monitoring microwave sensor can be automatically adjusted for greater accuracy in measuring intra-body glucose levels. In an example, a wearable glucose-monitoring microwave sensor can include one or more actuators which automatically adjust parameters of a resonator for greater accuracy in measuring intra-body glucose levels. In an example, automatic adjustment of a resonator can occur when a particular person first wears a device in order to customize the device to the person's specific anatomy and/or physiology. In an example, automatic adjustment of a resonator can occur each time the same person wears a device in order to optimize the device for specific way in which the device is worn (each time that it is worn). In an example, automatic adjustment of a resonator can occur in real time as a person is wearing a device based on changing physiological and environmental factors.

In an example, the position of one or more resonators can be automatically adjusted by one or more actuators in order to maintain a uniform distance from the surface of a person's body. In an example, the position of one or more resonators can be automatically adjusted by one or more actuators in order to maintain a consistent location on the surface of a person's body. In an example, the one or more parameters of a resonator in a wearable glucose-monitoring microwave sensor that are automatically adjusted can be selected from the group consisting of: split (or gap) distance; inter-resonator distance in an array, matrix, or series of resonators; distance between a resonator and the surface of a person's body; angle between a resonator and the surface of a person's body; orientation of a resonator relative to the surface of a person's body; location of a resonator on a person's body (relative to anatomical landmarks such as vasculature); frequency of microwave energy emitted; distance between a resonator and a microwave energy emitter or receiver; orientation of a resonator relative to a microwave energy emitter or receiver; and curvature of an array, matrix, or series of resonators.

In an example, the location, size, or shape of one or more resonators in a wearable glucose-monitoring microwave sensor can be automatically adjusted by one or more actuators in response to changes in temperature. In an example, the location, size, or shape of one or more resonators in a wearable glucose-monitoring microwave sensor can be automatically adjusted by one or more actuators in response to changes in moisture. In an example, the one or more parameters of a resonator in a wearable glucose-monitoring microwave sensor can be automatically adjusted based on one or more physiological and environment factors selected from the group consisting of: body motion or configuration; body moisture level and/or ambient humidity; body temperature and/or ambient temperature; food consumption; exercise; geographic location; and ambient electromagnetic activity.

In an example, a wearable glucose-monitoring microwave sensor can be incorporated into a wrist band, smart watch, or bracelet. In an example, a wearable glucose-monitoring microwave sensor can be incorporated into a finger ring. In an example, a wearable glucose-monitoring microwave sensor can be incorporated into an arm band. In an example, a wearable glucose-monitoring microwave sensor can be incorporated into an ear bud, ear plug, hearing aid, earphone, headphone, or earring. In an example, a wearable glucose-monitoring microwave sensor can be incorporated into the cuff and/or sleeve of a shirt. In an example, a wearable glucose-monitoring microwave sensor can be incorporated into the cuff and/or leg of a pair of pants or shorts. In an example, a wearable glucose-monitoring microwave sensor can be incorporated into a wearable patch or tattoo.

In an example, a wearable glucose-monitoring microwave sensor can be incorporated into the fabric of an article of clothing. In an example, electroconductive threads or yarns can function as microwave energy emitters and/or receivers. In an example, electroconductive thread or yarns in an article of clothing can expose body tissue and/or fluid to an electromagnetic field and the interaction of this body tissue and/or fluid with the electromagnetic fluid can be used to measure intra-body glucose levels. In an example, a wearable microwave sensor can be integrated into the fabric of a shirt, pair of pants or shorts, undershirt, underpants, sock, or belt. In an example, an electromagnetically-functional shirt, pair of pants or short, undershirt, underpants, sock, or belt can function as a wearable glucose-monitoring microwave sensor. In an example, an electromagnetically-functional shirt, pair of pants or short, undershirt, underpants, sock, or belt can function as the glucose-monitoring component of a closed-loop system for monitoring and managing intra-body glucose levels.

In an example, a first electroconductive thread or yarn in a woven fabric can function as a microwave energy emitter and a second electroconductive thread of yarn in a woven fabric can function as a microwave energy receiver. In an example, a series of resonators can be woven into fabric between the first electroconductive thread and the second electroconductive thread. In an example, a fabric woven from first and second electroconductive threads can create an electromagnetic field which can be used to measure the permittivity of nearby body tissue and/or fluid which, in turn, can be used to estimate intra-body glucose levels. In an example, a fabric comprised of electroconductive threads and electromagnetic resonators between electroconductive threads can create an electromagnetic field which can be used to measure the permittivity of nearby body tissue and/or fluid which, in turn, can be used to estimate intra-body glucose levels.

In an example, a wearable glucose-monitoring microwave sensor can be worn on a person's finger, hand, wrist, lower arm, or upper arm. In an example, a wearable glucose-monitoring microwave sensor can be worn on, in, or around a person's ear. In an example, a wearable glucose-monitoring microwave sensor can be worn on, in, or around a person's torso. In an example, a wearable glucose-monitoring microwave sensor can be incorporated into a piece of jewelry or clothing accessory. In an example, a wearable glucose-monitoring microwave sensor can be incorporated into a finger ring, watch, wrist band, bracelet, armband, necklace, or earring. In an example, a wearable glucose-monitoring microwave sensor can be incorporated into an article of clothing. In an example, a wearable glucose-monitoring microwave sensor can be incorporated into a glove, shirt, undershirt, pair of pants, shorts, undershorts, sock, shoe, belt, or eyeglasses.

In an example, a glucose monitoring and managing system can comprise: a wearable glucose-monitoring microwave sensor which collects data which is used to measure a person's intra-body glucose levels; and a wearable or implanted pump which delivers a glucose-level-modifying substance into the person's body based on data collected by the wearable glucose-monitoring microwave sensor. In an example, a wearable glucose-monitoring microwave sensor which monitors intra-body glucose levels and a wearable or implanted insulin pump which modifies intra-body glucose levels can together comprise a closed-loop system for automatically measuring and managing intra-body glucose levels. In an example, a wearable glucose-monitoring microwave sensor which monitors intra-body glucose levels and a wearable or implanted insulin pump which modifies intra-body glucose levels can together comprise a closed-loop system for automatically maintaining glucose levels within a selected range.

In an example, a pump which delivers a glucose-level-modifying substance into a person's body can be worn on the person's body. In an example, a pump which delivers a glucose-level-modifying substance into a person's body can be implanted within the person's body. In an example, a pump can dispense a substance which modifies the metabolism of intra-body glucose. In an example, a pump can dispense a substance which directly modifies the level of intra-body glucose. In an example, a pump can dispense insulin. In an example, a wearable glucose-monitoring microwave sensor and a wearable or implanted pump can be in wireless communication with each other. In an example, a wearable glucose-monitoring microwave sensor and a wearable or implanted insulin pump can be indirectly and wirelessly linked to each other by each having wireless communication with a common (albeit physically separate) data processing component.

In an example, In an example, a glucose monitoring and managing system can further comprise a microwave vector network analyzer which analyzes how the transmission and/or reflection of microwave energy is affected by interaction with nearby body tissue and/or fluid.

In an example, a glucose monitoring and managing system can further comprise one or more other sensors selected from the group consisting of: accelerometer, gyroscope, other inertial sensor, goniometer, bend sensor, stretch sensor, pressure sensor, GPS sensor, jaw motion sensor, and other motion sensor. In an example, one or more of these other sensors can detect when a person is eating food. In an example, detection of food consumption can trigger (a higher level of) operation of a wearable glucose-monitoring microwave sensor. In an example, a glucose monitoring and managing system can further comprise one or more of these other sensors selected from the group consisting of: microphone, swallow sensor, chewing sensor, ultrasound sensor, and other sound energy sensor. In an example, one or more of these other sensors can detect when a person is eating food. In an example, detection of food consumption can trigger (a higher level of) operation of a wearable glucose-monitoring microwave sensor.

In an example, a glucose monitoring and managing system can further comprise one or more of these other sensors selected from the group consisting of: camera, spectroscopic sensor, near-infrared sensor, infrared sensor, optoelectronic sensor, and other light energy sensor. In an example, one or more of these other sensors can detect when a person is eating food. In an example, detection of food consumption can trigger (a higher level of) operation of a wearable glucose-monitoring microwave sensor. In an example, a glucose monitoring and managing system can further comprise one or more of these other sensors selected from the group consisting of: EEG sensor, EMG sensor, other neuromuscular activity sensor, the electromagnetic brain activity sensor, skin impedance sensor, skin conductivity sensor, and other electromagnetic energy sensor. In an example, one or more of these other sensors can detect when a person is eating food. In an example, detection of food consumption can trigger (a higher level of) operation of a wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a motion sensor which collects data which is analyzed to detects changes in the configuration, position, and/or orientation of the microwave sensor relative to the person's body and/or changes in the person's body movement speed, acceleration, orientation, or direction; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a thermal energy sensor which collects data which is analyzed to detect changes in a person's body temperature and/or environmental temperature; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a moisture sensor which collects data which is analyzed to detect changes in a person's body moisture level and/or environmental moisture level; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a geographic position sensor which collects data which is analyzed to detect changes in geographic location and/or proximity to a place that is associated with food consumption; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

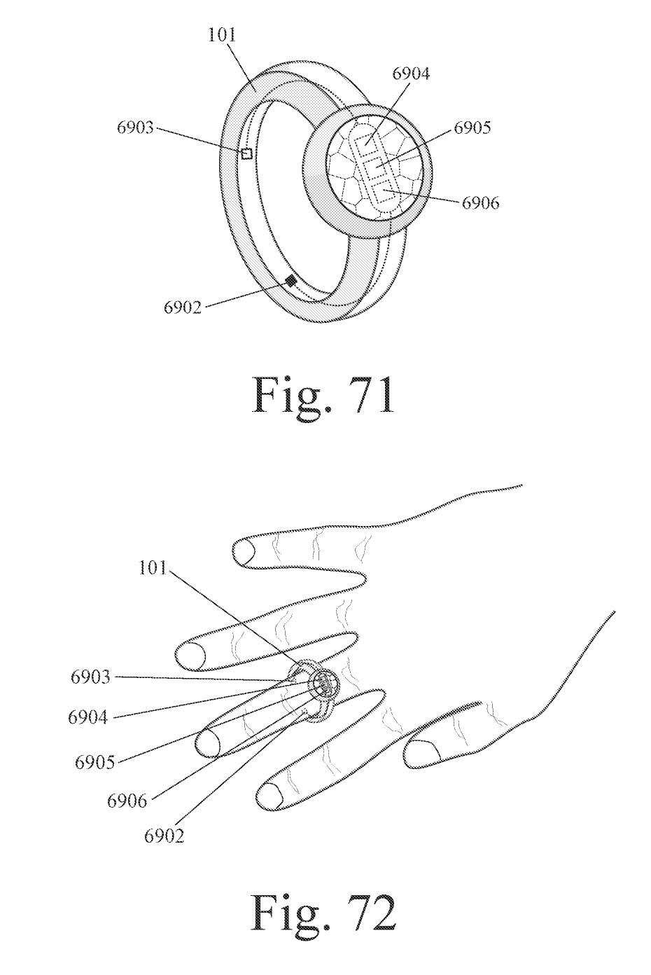

FIGS. 71 and 72 show an example of how this invention can be embodied in a finger-worn device for monitoring a person's food consumption comprising—a ring 101 which is configured to be worn on a person's finger, wherein this ring further comprises: an electromagnetic energy emitter 6902 which is configured to emit electromagnetic energy into the person's finger tissue at a first location; an electromagnetic energy receiver 6903 which is configured to receive electromagnetic energy from the person's finger tissue at a second location, wherein parameters or patterns of the received electromagnetic energy are changed by the person's consumption of food and analyzed to monitor the person's food consumption; a power source 6904; a data processor 6905; and a data transmitter 6906. FIG. 71 shows this finger ring by itself. FIG. 72 shows this finger ring being worn on a person's finger. Relevant example variations which are discussed in other portions of this disclosure or the family of priority-related disclosures can also be applied to the embodiment shown here in FIGS. 71 and 72.

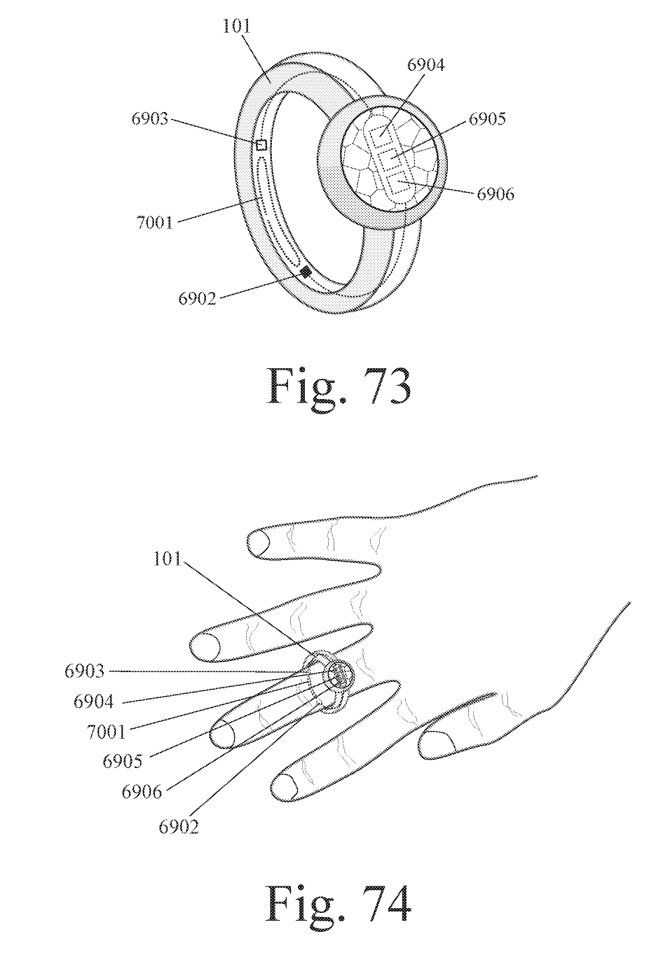

FIGS. 73 and 74 show another example of how this invention can be embodied in a finger-worn device for monitoring a person's food consumption comprising—a ring 101 which is configured to be worn on a person's finger, wherein this ring further comprises: an electromagnetic energy emitter 6902 which is configured to emit electromagnetic energy into the person's finger tissue at a first location; an electromagnetic energy receiver 6903 which is configured to receive electromagnetic energy from the person's finger tissue at a second location, wherein parameters or patterns of the received electromagnetic energy are changed by the person's consumption of food and analyzed to monitor the person's food consumption; an electromagnetic energy resonator 7001 between the electromagnetic energy emitter and the electromagnetic energy receiver; a power source 6904; a data processor 6905; and a data transmitter 6906. FIG. 73 shows this finger ring by itself. FIG. 74 shows this finger ring being worn on a person's finger. Relevant example variations which are discussed in other portions of this disclosure or the family of priority-related disclosures can also be applied to the embodiment shown here in FIGS. 73 and 74.

In an example, this invention can be embodied in a finger-worn device for monitoring a person's food consumption comprising: a ring which is configured to be worn on a person's finger, wherein this ring further comprises—an electromagnetic energy sensor which is configured to measure parameters or patterns of electromagnetic energy transmitted through the person's finger tissue, wherein these parameters or patterns of electromagnetic energy are changed by the person's consumption of food and analyzed to monitor the person's food consumption; a power source; a data processor; and a data transmitter. In an example, an electromagnetic energy sensor can be an electromagnetic impedance sensor which measures the impedance of a person's finger tissue. In an example, an electromagnetic energy sensor can be an electromagnetic resistance sensor which measures the resistance, however futile, of a person's finger tissue. In an example, an electromagnetic energy sensor can be an electromagnetic conductivity sensor which measures the conductivity of a person's finger tissue.

In an example, this invention can be embodied in a finger-worn device for monitoring a person's food consumption comprising—a ring which is configured to be worn on a person's finger, wherein this ring further comprises: an electromagnetic energy emitter which is configured to emit electromagnetic energy into the person's finger tissue at a first location; an electromagnetic energy receiver which is configured to receive electromagnetic energy from the person's finger tissue at a second location, wherein parameters or patterns of the received electromagnetic energy are changed by the person's consumption of food and analyzed to monitor the person's food consumption; a power source; a data processor; and a data transmitter.

In an example, parameters or patterns of the electromagnetic energy can include the impedance of a person's finger tissue. In an example, parameters or patterns of the electromagnetic energy can include the resistance of a person's finger tissue. In an example, parameters or patterns of the electromagnetic energy can include the conductivity of a person's finger tissue. In an example, parameters or patterns of the electromagnetic energy can include the permittivity of a person's finger tissue. In an example, a device can further comprise a plurality of electromagnetic energy emitters or electromagnetic energy receivers distributed around the circumference of the finger ring.

In an example, a device can further comprise an electromagnetic resonator between an electromagnetic energy emitter and an electromagnetic energy receiver. In an example, an electromagnetic resonator can comprise a split ring. In an example, an electromagnetic resonator can comprise two or more nested rings. In an example, an electromagnetic resonator can comprise two or more stacked rings. In an example, an electromagnetic resonator can comprise a spiral. In an example, this invention can be embodied in a finger-worn device for monitoring a person's food consumption comprising—a ring which is configured to be worn on a person's finger, wherein this ring further comprises: an electromagnetic energy emitter which is configured to emit electromagnetic energy at a first location; an electromagnetic energy receiver which is configured to receive electromagnetic energy at a second location, wherein parameters or patterns of the received electromagnetic energy are changed by the person's consumption of food and analyzed to monitor the person's food consumption; an electromagnetic energy resonator between the electromagnetic energy emitter and the electromagnetic energy receiver; a power source; a data processor; and a data transmitter.

I claim:

1. A finger-worn device for monitoring a person's food consumption comprising:
a ring which is configured to be worn on a person's finger, wherein this ring further comprises:
an electromagnetic energy sensor which is configured to measure parameters or patterns of electromagnetic energy transmitted through the person's finger tissue, wherein these parameters or patterns of electromagnetic energy are changed by the person's consumption of food and analyzed to monitor the person's food consumption;
a power source;
a data processor; and
a data transmitter.

2. The device in claim 1 wherein the electromagnetic energy sensor is an electromagnetic impedance sensor which measures the impedance of the person's finger tissue.

3. The device in claim 1 wherein the electromagnetic energy sensor is an electromagnetic resistance sensor which measures the resistance of the person's finger tissue.

4. The device in claim 1 wherein the electromagnetic energy sensor is an electromagnetic conductivity sensor which measures the conductivity of the person's finger tissue.

5. A finger-worn device for monitoring a person's food consumption comprising:
a ring which is configured to be worn on a person's finger, wherein this ring further comprises:
an electromagnetic energy emitter which is configured to emit electromagnetic energy into the person's finger tissue at a first location;
an electromagnetic energy receiver which is configured to receive electromagnetic energy from the person's finger tissue at a second location, wherein parameters or patterns of the received electromagnetic energy are changed by the person's consumption of food and analyzed to monitor the person's food consumption;
a power source;
a data processor; and
a data transmitter.

6. The device in claim 5 wherein the parameters or patterns of the electromagnetic energy include the impedance of the person's finger tissue.

7. The device in claim 5 wherein the parameters or patterns of the electromagnetic energy include the resistance of the person's finger tissue.

8. The device in claim 5 wherein the parameters or patterns of the electromagnetic energy include the conductivity of the person's finger tissue.

9. The device in claim 5 wherein the parameters or patterns of the electromagnetic energy include the permittivity of the person's finger tissue.

10. The device in claim 5 wherein the device further comprises a plurality of electromagnetic energy emitters or electromagnetic energy receivers distributed around the circumference of the finger ring.

11. The device in claim 5 wherein the finger ring further comprises an electromagnetic resonator between the electromagnetic energy emitter and the electromagnetic energy receiver.

12. The device in claim 11 wherein the electromagnetic resonator comprises a split ring.

13. The device in claim 11 wherein the electromagnetic resonator comprises two or more nested rings.

14. The device in claim 11 wherein the electromagnetic resonator comprises two or more stacked rings.

15. The device in claim 11 wherein the electromagnetic resonator comprises a spiral.

16. A finger-worn device for monitoring a person's food consumption comprising:
a ring which is configured to be worn on a person's finger, wherein this ring further comprises:
an electromagnetic energy emitter which is configured to emit electromagnetic energy at a first location;
an electromagnetic energy receiver which is configured to receive electromagnetic energy at a second location, wherein parameters or patterns of the received electromagnetic energy are changed by the person's consumption of food and analyzed to monitor the person's food consumption;
an electromagnetic energy resonator between the electromagnetic energy emitter and the electromagnetic energy receiver;
a power source;
a data processor; and
a data transmitter.

17. The device in claim 16 wherein the electromagnetic resonator comprises a split ring.

18. The device in claim 16 wherein the electromagnetic resonator comprises two or more nested rings.

19. The device in claim 16 wherein the electromagnetic resonator comprises two or more stacked rings.

20. The device in claim 16 wherein the electromagnetic resonator comprises a spiral.
